SEQUENCE LISTING (61 sequences)

SEQ ID NO: 1 - protein, 224 aa, synthetic construct
  REGION 1..224: note = anti-CD40(G28.5)-Flag-VL-pCR3
DIVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 483 aa, synthetic construct
  REGION 1..483: note = anti-CD40(G28.5)-Flag-VH-N297A-TNC-pCR3
DIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 3 - protein, 446 aa, synthetic construct
  REGION 1..446: note = anti-CD40(G28.5)-Flag-VH-IgG1(A+RGY)-pCR3
DIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 4 - protein, 687 aa, synthetic construct
  REGION 1..687: note = anti-CD40(G28.5)-Flag-VH-N297A-scFv-anti-CD40(G28.5 )-pCR3
DIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIKGGGGSGGGGSGGGGSDIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSS

SEQ ID NO: 5 - protein, 219 aa, synthetic construct
  REGION 1..219: note = anti-CD40(Lob7.4)-Flag-VL-pCR3
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 493 aa, synthetic construct
  REGION 1..493: note = anti-CD40(Lob7.4)-Flag-VH-N297A-TNC-pCR3
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 7 - protein, 456 aa, synthetic construct
  REGION 1..456: note = anti-CD40(Lob7.4)-Flag-VH-IgG1(A+RGY)-pCR3
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 8 - protein, 706 aa, synthetic construct
  REGION 1..706: note = anti-CD40(Lob7.4)-Flag-VH-N297A-scFv-anti-CD40(Lob7 .4)-pCR3
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSRSSTKGPKLEEGEFSEAQLDIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 9 - protein, 224 aa, synthetic construct
  REGION 1..224: note = anti-CD40(Mitazalimab)-Flag-VL-pCR3
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 490 aa, synthetic construct
  REGION 1..490: note = anti-CD40(Mitazalimab)-Flag-VH-N297A-TNC-pCR3
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 11 - protein, 453 aa, synthetic construct
  REGION 1..453: note = anti-CD40(Mitazalimab)-Flag-VH-IgG1(A+RGY-pCR3
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 12 - protein, 705 aa, synthetic construct
  REGION 1..705: note = anti-CD40(Mitazalimab)-Flag-VH-N297A-scFv-anti-CD40 (Mitazalimab)-pCR3
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSRSSTKGPKLEEGEFSEAQLQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 13 - protein, 220 aa, synthetic construct
  REGION 1..220: note = anti-CD40(Apexigen)-Flag-VL-pCR3
DIVMTQTPSSASEPVGGTVTIKCQASQSISSRLAWYQQKPGQPPKLLIYRASTLASGVPSRFKGSGSGTEFTLTISDLECADAATYYCQCTGYGISWPIGGGTEVVVKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 489 aa, synthetic construct
  REGION 1..489: note = anti-CD40(Apexigen)-Flag-VH-N297A-TNC-pCR3
QSLEESGGDLVKPGASLTLTCTASGFSFSSTYVCWVRQAPGKGLEWIACIYTGDGTNYSASWAKGRFTISKPSSTTVTLQMTSLTPADTATYFCARPDITYGFAINFWGPGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 15 - protein, 452 aa, synthetic construct
  REGION 1..452: note = anti-CD40(Apexigen)-Flag-VH-(A+RGY)-pCR3
QSLEESGGDLVKPGASLTLTCTASGFSFSSTYVCWVRQAPGKGLEWIACIYTGDGTNYSASWAKGRFTISKPSSTTVTLQMTSLTPADTATYFCARPDITYGFAINFWGPGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 16 - protein, 699 aa, synthetic construct
  REGION 1..699: note = anti-CD40(Apexigen)-Flag-VH-N297A-scFv-anti-CD40(Ap exigen)-pCR3
QSLEESGGDLVKPGASLTLTCTASGFSFSSTYVCWVRQAPGKGLEWIACIYTGDGTNYSASWAKGRFTISKPSSTTVTLQMTSLTPADTATYFCARPDITYGFAINFWGPGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQSLEESGGDLVKPGASLTLTCTASGFSFSSTYVCWVRQAPGKGLEWIACIYTGDGTNYSASWAKGRFTISKPSSTTVTLQMTSLTPADTATYFCARPDITYGFAINFWGPGTLVTVSSRSSTKGPKLEEGEFSEAQLDIVMTQTPSSASEPVGGTVTIKCQASQSISSRLAWYQQKPGQPPKLLIYRASTLASGVPSRFKGSGSGTEFTLTISDLECADAATYYCQCTGYGISWPIGGGTEVVVK

SEQ ID NO: 17 - protein, 219 aa, synthetic construct
  REGION 1..219: note = anti-CD40(Selicrelumab)-Flag-VL-pCR3
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 497 aa, synthetic construct
  REGION 1..497: note = anti-CD40(Selicrelumab)-Flag-VH-N297A-TNC-pCR3
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 19 - protein, 460 aa, synthetic construct
  REGION 1..460: note = anti-CD40(Selicrelumab)-Flag-VH-(A+RGY)-pCR3
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 20 - protein, 714 aa, synthetic construct
  REGION 1..714: note = anti-CD40(Selicrelumab)-Flag-VH-scFv- anti-CD40(Selicrelumab)-pCR3
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSRSSTKGPKLEEGEFSEAQLDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 21 - protein, 224 aa, synthetic construct
  REGION 1..224: note = anti-CD40(V12)VHH-Flag-LC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 388 aa, synthetic construct
  REGION 1..388: note = anti-CD40(V12)VHH(2x)-Flag-LC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQFEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 502 aa, synthetic construct
  REGION 1..502: note = anti-CD40(V12)-VHH(3x)-Flag-LC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 446 aa, synthetic construct
  REGION 1..446: note = anti-CD40(V12)-VHH-Flag-N297A-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 610 aa, synthetic construct
  REGION 1..610: note = anti-CD40(V12)-VHH(2x)-Flag-N297A-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 724 aa, synthetic construct
  REGION 1..724: note = anti-CD40(V12)-VHH(3x)-N297A-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 157 aa, synthetic construct
  REGION 1..157: note = anti-CD40(V12)VHH-Flag-TNC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSDYKDDDDKDIACGCAAAPDIKDLLSRLEELEGLVSSFREQGTG

SEQ ID NO: 28 - protein, 321 aa, synthetic construct
  REGION 1..321: note = anti-CD40(V12)VHH(2x)-TNC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLDYKDDDDKDIACGCAAAPDIKDLLSRLEELEGLVSSLREQGTG

SEQ ID NO: 29 - protein, 435 aa, synthetic construct
  REGION 1..435: note = anti-CD40(V12)(3x)-Flag-TNC-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSDYKDDDDKDIACGCAAAPDIKDLLSRLEELEGLVSSLREQGTG

SEQ ID NO: 30 - protein, 26 aa, synthetic construct
  REGION 1..26: note = Leader sequence
MNFGFSLIFLVLVLKGVQCEVKLVPR

SEQ ID NO: 31 - protein, 352 aa, synthetic construct
  REGION 1..352: note = anti-CD40(V12)VHH-Fc(DANA)-Flag-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLE

SEQ ID NO: 32 - protein, 526 aa, synthetic construct
  REGION 1..526: note = anti-CD40(V12)VHH(2x)-Fc(DANA)-2xFlag-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLGTKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDYKDDDDKEFDYKDDDDKLE

SEQ ID NO: 33 - protein, 630 aa, synthetic construct
  REGION 1..630: note = anti-CD40(V12)VHH(3x)-Fc(DANA)-Flag-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLE

SEQ ID NO: 34 - protein, 464 aa, synthetic construct
  REGION 1..464: note = anti-CD40(V12)VHH-Fc(DANA)-Flag-anti-CD40(V12)VHH-p CR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLEQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 35 - protein, 742 aa, synthetic construct
  REGION 1..742: note = anti-CD40(V12)VHH(3x)-Fc(DANA)-Flag-anti-CD40(V12)V HH-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLEQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 36 - protein, 224 aa, synthetic construct
  REGION 1..224: note = anti-CD40(V12)VHH-Flag-FAB1(1-108)-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 37 - protein, 388 aa, synthetic construct
  REGION 1..388: note = anti-CD40(V12)VHH(2x)-Flag-FAB1(1-108)-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 38 - protein, 502 aa, synthetic construct
  REGION 1..502: note = anti-CD40(V12)VHH(3x)-Flag-FAB1(1-108)-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 39 - protein, 336 aa, synthetic construct
  REGION 1..336: note = anti-CD40(V12)VHH-Flag-FAB1(1-108)-anti-CD40(V12)VH H-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 40 - protein, 502 aa, synthetic construct
  REGION 1..502: note = anti-CD40(V12) VHH(2x)-Flag-FAB1 (1-108)-anti-CD40(V12) VHH-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTLEQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 41 - protein, 616 aa, synthetic construct
  REGION 1..616: note = anti-CD40(V12) VHH(3x)-Flag-FAB1 (1-108)-anti-CD40(V12) VHH-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSRSGGGGSGGGGSGGGGSGGGGSGGGGSQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSGGGGSGGGGSGGGGSGGGGSQLQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTLEQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 42 - protein, 271 aa, synthetic construct
  REGION 1..271: note = anti-CD40(V12)-VHH-Flag-TNC-anti-CD40(V12)-VHH-pCR3
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGSDYKDDDDKDIACGCAAAPDIKDLLSRLEELEGLVSSFREQGTGVEQVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 43 - protein, 224 aa, synthetic construct
  REGION 1..224: note = anti-CD40(15B8)-Flag-VL-pCR3
DIVMTQSPLSLSVAPGQPASISCKSSQSLLESYGETYLYWYLQKPGQPPQLLIYAVFKRFSGVPDRFSGSGSGTDFTLKISRVEAEDVGVYYCMQSMQLPLTFGGGTKVEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 493 aa, synthetic construct
  REGION 1..493: note = anti-CD40(15B8)-Flag-VH-N297A-TNC-pCR3
QVQLQESGGGVVQPGRSLRLSCAASGFTFNNFGIHWVRQAPGKGLEWVAVISYDGSDKYYADSVKGRFTISRDNSKNTLNLQMNSLRAEDTAVYYCARDRRYYYHYYGMDVWGQGTMVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 45 - protein, 456 aa, synthetic construct
  REGION 1..456: note = anti-CD40(15B8)-Flag-VH-IgG1(A+RGY)-pCR3
QVQLQESGGGVVQPGRSLRLSCAASGFTFNNFGIHWVRQAPGKGLEWVAVISYDGSDKYYADSVKGRFTISRDNSKNTLNLQMNSLRAEDTAVYYCARDRRYYYHYYGMDVWGQGTMVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 46 - protein, 711 aa, synthetic construct
  REGION 1..711: note = anti-CD40(15B8)-Flag-VH-N297A-scFv-anti-CD40(15B8)- pCR3
QVQLQESGGGVVQPGRSLRLSCAASGFTFNNFGIHWVRQAPGKGLEWVAVISYDGSDKYYADSVKGRFTISRDNSKNTLNLQMNSLRAEDTAVYYCARDRRYYYHYYGMDVWGQGTMVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQVQLQESGGGVVQPGRSLRLSCAASGFTFNNFGIHWVRQAPGKGLEWVAVISYDGSDKYYADSVKGRFTISRDNSKNTLNLQMNSLRAEDTAVYYCARDRRYYYHYYGMDVWGQGTMVTVSSRSSTKGPKLEEGEFSEAQLDIVMTQSPLSLSVAPGQPASISCKSSQSLLESYGETYLYWYLQKPGQPPQLLIYAVFKRFSGVPDRFSGSGSGTDFTLKISRVEAEDVGVYYCMQSMQLPLTFGGGTKVEIK

SEQ ID NO: 47 - protein, 219 aa, synthetic construct
  REGION 1..219: note = anti-CD40(24-31)-Flag-VL-pCR3
DIVMTQSPSFLSASVGDRVTITCKASQNVITAVAWYQQKPGKSPKLLIYSASNRYTGVPDRFSGSGSGTDFTLTISSLQPEDFADYFCQQYNSYPYTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 491 aa, synthetic construct
  REGION 1..491: note = anti-CD40(24-31)-Flag-VH-N297A-TNC-pCR3
EVQLQESGPGLVKPSETLSLTCTVSGDSITNGFWIWIRKPPGNKLEYMGYISYSGSTYYNPSLKSRISISRDTSKNQFSLKLSSVTAADTGVYYCACRSYGRTPYYFDFWGQGTTLTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 49 - protein, 454 aa, synthetic construct
  REGION 1..454: note = anti-CD40(24-31)-Flag-VH-IgG1(A+RGY)-pCR3
EVQLQESGPGLVKPSETLSLTCTVSGDSITNGFWIWIRKPPGNKLEYMGYISYSGSTYYNPSLKSRISISRDTSKNQFSLKLSSVTAADTGVYYCACRSYGRTPYYFDFWGQGTTLTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 50 - protein, 702 aa, synthetic construct
  REGION 1..702: note = anti-CD40(24-31)-Flag-VH-N297A-scFv-anti-CD40(24-31 )-pCR3
EVQLQESGPGLVKPSETLSLTCTVSGDSITNGFWIWIRKPPGNKLEYMGYISYSGSTYYNPSLKSRISISRDTSKNQFSLKLSSVTAADTGVYYCACRSYGRTPYYFDFWGQGTTLTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLQESGPGLVKPSETLSLTCTVSGDSITNGFWIWIRKPPGNKLEYMGYISYSGSTYYNPSLKSRISISRDTSKNQFSLKLSSVTAADTGVYYCACRSYGRTPYYFDFWGQGTTLTVSSRSSTKGPKLEEGEFSEAQLDIVMTQSPSFLSASVGDRVTITCKASQNVITAVAWYQQKPGKSPKLLIYSASNRYTGVPDRFSGSGSGTDFTLTISSLQPEDFADYFCQQYNSYPYTFGGGTKLEIK

SEQ ID NO: 51 - protein, 218 aa, synthetic construct
  REGION 1..218: note = anti-CD40(F5)-Flag-VL-pCR3
DIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFQGTKVEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 495 aa, synthetic construct
  REGION 1..495: note = anti-CD40(F5)-Flag-VH-N297A-TNC-pCR3
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 53 - protein, 458 aa, synthetic construct
  REGION 1..458: note = anti-CD40(F5)-Flag-VH-IgG1(A+RGY)-pCR3
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 54 - protein, 709 aa, synthetic construct
  REGION 1..709: note = anti-CD40(F5)-Flag-VH-N297A-scFv-anti-CD40(F5)-pCR3
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSRSSTKGPKLEEGEFSEAQLDIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFQGTKVEIK

SEQ ID NO: 55 - protein, 233 aa, synthetic construct
  REGION 1..223: note = anti-CD40(V15t)VHH-Flag-LC-pCR3
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 455 aa, synthetic construct
  REGION 1..455: note = anti-CD40(V15t)-VHH-Flag-N297A-pCR3
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 657 aa, synthetic construct
  REGION 1..657: note = anti-CD40(V15t)VHH(3x)-Fc(DANA)-Flag-pCR3
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSRSGGGGSGGGGSGGGGSGGGGSGGGGSEVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGGGGSGGGGSGGGGSGGGGSGGGGSQLEVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLE

SEQ ID NO: 58 - protein, 462 aa, synthetic construct
  REGION 1..462: note = anti-CD40(V15t)(3x)-Flag-TNC-pCR3
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSRSGGGGSGGGGSGGGGSGGGGSGGGGSEVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGGGGSGGGGSGGGGSGGGGSGGGGSQLEVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGSDYKDDDDKDIACGCAAAPDIKDLLSRLEELEGLVSSFREQGTG

SEQ ID NO: 59 - protein, 8 aa, synthetic construct
  REGION 1..8: note = Flag tag
DYKDDDDK

SEQ ID NO: 60 - protein, 35 aa, synthetic construct
  REGION 1..35: note = Tenascin C (TNC) trimerization domain
DIACGCAAAPDIKDLLSRLEELEGLVSSLREQGTG

SEQ ID NO: 61 - protein, 8 aa, synthetic construct
  REGION 1..8: note = Flag tag
DYKDDDDK

(19) United States
(12) Patent Application Publication
(10) Pub. No.: US 2026/0092124 A1
(43) Pub. Date: Apr. 2, 2026

(54) ANTI-CD40 ANTIBODY CONSTRUCTS WITH HIGH INTRINSIC AGONISM

(71) Applicant: JULIUS-MAXIMILIANS-UNIVERSITÄT WÜRZBURG, Würzburg (DE)

(72) Inventors: Harald Wajant, Kist (DE); Isabell Lang, Neuendorf (DE); Daniela Siegmund, Ilshofen (DE); Andreas Beilhack, Würzburg (DE)

(21) Appl. No.: 19/113,622

(22) PCT Filed: Sep. 22, 2023
(86) PCT No.: PCT/EP2023/076200

§ 371 Date: Mar. 20, 2025

(30) Foreign Application Priority Data
Sep. 22, 2022 (EP) 22197095.7

(51) Int. Cl. C07K 16/28

(52) U.S. Cl. CPC C07K 16/2878; C07K 2317/33; C07K 2317/35; C07K 2317/569; C07K 2317/72; C07K 2317/75

(57) ABSTRACT

The present invention relates to oligomeric and oligovalent anti-CD40 antibody variants displaying high intrinsic, thus FcγR-independent, agonistic activity. The invention more specifically relates to tetra-, penta-, hexa-, hepta-, octa-, nona-, deca- and docevalent or even higher valency antibody constructs with FcγR-independent activity composed of CD40-specific Fab- and/or scFv domains and/or sdAb domains and an dimeric, trimeric, tetrameric oligomerization scaffold preferentially Fc, IgG molecule or trimerization domain of TNC. The invention also relates to the enforced oligomerization of several anti-CD40 IgG molecules, that can be achieved in a stoichiometric fashion by genetic engineering, that confers strong FcγR-independent agonism, and to the fact that the idiotype of the anti-CD40 antibody has no major effect on the autonomous agonism of oligovalent anti-CD40 variants. The invention also relates to methods of producing such oligomeric and oligovalent anti-CD40 antibody constructs, pharmaceutical compositions comprising the same, as well as their uses for treating cancer.

### FIELD OF THE INVENTION

The present invention relates to oligomeric and oligovalent anti-CD40 antibody variants displaying high intrinsic, thus FcγR-independent, agonistic activity. The invention more specifically relates to tetra-, hexa- and octavalent antibody constructs composed of CD40-specific Fab- and scFv domains with FcγR-independent activity. The invention also relates to the enforced oligomerization of several anti-CD40 molecules, that can be achieved in a stoichiometric fashion by genetic engineering, that confers strong FcγR-independent agonism, and to the fact that the idiotype of the anti-CD40 antibody has no major effect on the autonomous agonism of oligovalent anti-CD40 variants. The invention also relates to methods of producing such oligomeric and oligovalent anti-CD40 antibody constructs, pharmaceutical compositions comprising the same, as well as their uses for treating cancer.

### BACKGROUND

By far the majority of preclinical and clinical studies aimed at activating CD40 in tumor treatment or as adjuvant in the vaccination against pathogens use/used agonistic anti-CD40 antibodies (Vonderheide 2020; Li and Wang 2020, Bullock 2021). With respect to the agonism of anti-CD40 antibodies, it is, however, extremely important to distinguish between antibody-intrinsic autonomous agonism which is evident without binding of the antibody to Fcγ receptors (FcγRs) and a conditional type of CD40 agonism which become only apparent when the antibody has been bound to a cell expressed FcγR molecule. Thus, strictly and correctly speaking, anti-CD40 antibodies with FcγR-dependent agonism are no agonists, since their sole binding to CD40 does not have a receptor-stimulating effect. Rather, the complexes of these non-agonistic antibodies with other per se non-agonistic molecules (FcγRs, crosslinking antibodies etc.) display the agonistic effect. Unfortunately, this distinction has not been made in many published studies, thus the term “agonist” is used in the literature for both anti-CD40 antibodies with FcγR-dependent and FcγR-independent agonism, although this aspect may be absolutely critical for the interpretation of data obtained with anti-CD40 antibodies.

The majority of the anti-CD40 IgG antibodies considered as agonistic antibodies in the literature, indeed, have no relevant autonomous agonism and only act agonistic when bound to FcγRs or upon cross-linking with secondary antibodies or protein G (Wajant 2015). For example, a mutant of the “agonistic” anti-CD40-IgG1 ADC-1013 (Mitazalimab) harboring a N297Q mutation in the Fc part reducing glycosylation and FcγR binding lose its ability to engage dendritic cells but display restored agonism upon cross-linking with anti-human IgG (Mangsbo et al., 2015). Similarly, variants of the “agonistic” anti-CD40 antibody APX005M (Sotigalimab) lack agonism upon removal of the Fc domain but display enhanced agonism on B-cells upon introduction of the S267E mutation improving affinity for FcγRIIb (Filbert et al., 2021). Comparable results have also been reported for several other anti-CD40 antibodies including SEA-CD40 (Grilley-Olsen et al., 2018), G28.5 (Medler et al., 2019) and ChiLob-7/4 (Chowdhury et al., 2014).

For a few anti-CD40-IgGs significant FcγR-independent agonism have been reported. For example, Fab2 fragments of CP-870,893 (Selicrelumab) showed similar B-cell activation as the corresponding complete antibody and secondary crosslinking of the latter showed no relevant further increase in activity (Richmann and Vonderheide). Similar findings have been obtained for the anti-CD40 antibody CDX-1140 (Vitale et al., 2018). There is evidence that the isotype of an anti-CD40 antibody is of particular relevance for its agonistic activity. So, CP-870,893 and CDX-1140 are both of the IgG2 isotype and conversion of the anti-CD40-IgG1 ChiLob-4/7 into the IgG2 isotype come along with significant FcγR-independent agonism (White et al., 2015). Intriguingly, the agonistic activity of anti-CD40 hIgG2 antibodies could be assigned to isoform B of the hIgG2 isotype, which differs from the A isoform of the hIgG2 molecule in the formation of the disulfide bridges between the CH1 and CL domains, and which has thus a less flexible arrangement of the two Fab domains of the molecule. Anti-CD40 hIgG2 antibodies that have mutations that instruct only the formation of isoform A (e.g. HC-C233S) or isoform B (e.g. HC-C127S or LC-C214S/HC-C233S) have therefore no or increased FcγR-independent agonism compared to the parental hIgG2 molecule (White et al., 2015, Yu et al., 2020). However, the general relevance of IgG2 isotype and the IgG2B isoform for FcγR-independent CD40 agonism has been challenged by studies showing i) loss of agonism of deglycosylated CP-870,893-IgG2 having reduced affinity for FcγRIIb and ii) similar agonism of the two IgG2 isoforms (Dahan et al., 2016). Moreover, the FcγR-independent agonism of anti-CD40-hIgG2 or anti-CD40-hIgG2A antibodies, which sometimes are called “superagonists”, is still significantly lower than that of FcγR-bound anti-CD40 antibodies (Dahan et al., 2016; Yu et al., 2018). Thus, the presence of transfectants expressing high amounts of FcγRIIb, therefore conditions delivering the best chance that all CD40-occupying antibody molecules are also indeed bound to an FcγR molecule, uniformly result in strongly enhanced CD40 agonism (Yu et al., 2018).

In the past decade, extensive in vitro and in vivo studies have revealed that virtually every CD40-specific antibody displays agonistic activity if it is bound to Fcγ receptors (Li and Ravetch 2011; Wilson et al., 2011; Dahan et al., 2016; Yu et al., 2018; KUMS 2019/Nelke). The concrete FcγR type and its signal transduction properties are in this context irrelevant for the agonism of Fcγ-bound anti-CD40 antibodies, rather it seems to be that it is the sheer plasma membrane-linked mode of presentation which is necessary for the Fcγ-bound anti-CD40 antibody molecules to acquire agonism. Accordingly, it has been found that bispecific antibody variants that recognize a second plasma membrane-associated antigen in addition to CD40 show an up to 1000-fold increase in CD40-stimulating activity after binding to this second antigen (Yeh et al., 2019; Sum et al., 2021, Sum et al., 2022; Medler et al., 2019; Medler et al., 2022; Nelke et al., 2020, Salomon et al., 2022). As improving FcγR binding of antibodies by genetic engineering is state of the art, it appears trivial to achieve strong CD40 agonism in vivo by help of anti-CD40 antibodies with strong conditional FcγR-dependent agonism, however, the need for FcγR-binding comes along with several limitations. The expression levels of CD40 are typically quite high and FcγR-expressing immune cells can be of limited availability. Thus, one has to consider that in vivo only a fraction of CD40 molecules can be targeted by FcγR-bound anti-CD40 antibodies resulting in submaximal stimulation of CD40 activities. For example, anti-CD40 murine IgG1 antibodies significantly stimulate proliferation of B-cells from wild-type mice but not of FcγRIIB-deficient B cells. However, if FcγR-expressing transfectants are also added to such experiments, proliferation can be further increased by one to two orders of magnitude (Yu et al., 2018). Obviously, the physiological levels of FcγR expressed by B cells are insufficient to reach all CD40 molecules with an FcγR-bound anti-CD40 antibody. Another aspect that limits the utility of the FcγR-dependent agonism of conventional anti-CD40 antibodies for immune stimulation is the binding to activating FcγRs, e.g. FcγRIIIa. In such cases, the FcγR binding might not only result in CD40 agonism but may also trigger the killing of the CD40-expressing target cell by effector functions (e.g. ADCC, CDC) of the FcγR-expressing cells.

### DESCRIPTION OF THE INVENTION

Agonistic anti-CD40 antibodies widely differ in their features. It has been found that some anti-CD40 antibodies show only relevant agonism upon binding to FcγR+ immune cells, while other anti-CD40 antibodies already display intrinsic agonism dependent on the idio- and/or isotype. It is worth mentioning, however, that all these antibodies show a further increase in activity when cells overexpressing FcγRs are available, indicating i) that agonistic anti-CD40 antibody selection based on idio- or isotype is not sufficient to obtain antibodies enabling the maximal possible CD40 activation and ii) that the limited availability of endogenously expressed FcγRs can restrict the extent of CD40 activation, too. In view of the well documented fact that oligomerization of soluble CD40L trimers by genetic engineering strongly enhances their CD40-stimulatory ability, the inventors proved here the hypothesis that oligomeric and oligovalent anti-CD40 antibody variants generated by genetic engineering display high intrinsic, thus FcγR-independent, agonistic activity. The inventors generated tetra-, hexa- and dodecavalent variants of six anti-CD40 antibodies with widely differing features. All these variants, including those derived of antagonistic anti-CD40 antibodies, showed strongly enhanced CD40 agonism compared to their conventional counterparts. In most cases, the CD40 agonism reached the maximum response inducible by FcγR-bound anti-CD40 antibodies or membrane CD40L, the natural engager of CD40. In sum, the present data show that increasing the anti-CD40 antibody valency by genetic engineering regularly results in molecules with high autonomous agonism and level out the specific limitations of the parental antibodies.

The inventors have identified antibody constructs, including inter alia oligomeric and oligovalent anti-CD40 antibody variants displaying high intrinsic, thus FcγR-independent, agonistic activity. The inventors have found i) that the enforced oligomerization of several anti-CD40 molecules, that can be achieved in a stoichiometric fashion by genetic engineering, confers strong FcγR-independent agonism and ii) that the idiotype of the anti-CD40 antibody has no major effect on the autonomous agonism of oligovalent anti-CD40 variants.

According to the invention, these novel antibody-based CD40 agonists stimulate CD40 in a FcγR-independent manner, and it is expected that this advantage will translate into opening new fields of application, including clinical applications such as cancer therapy or vaccination against infection diseases.

Accordingly, the invention relates to the following preferred embodiments:
1. A multivalent anti-CD40 antibody construct comprising at least 4 antigen binding sites for CD40, wherein the antibody construct is not a natural IgA or IgM molecule.
2. The construct of item 1, wherein the construct comprises at least one IgG molecule containing two of said antigen binding sites for CD40.
3. The construct of item 1, wherein the construct comprises a modified IgG molecule, which has been modified by replacing each of the two variable domains of the heavy chain by at least one single-domain antibody (sdAb) containing one of said antigen binding sites for CD40, and by replacing each of the two variable domains of the light chain by at least one single-domain antibody (sdAb) containing one of said antigen binding sites for CD40.
4. The construct of item 2 or 3, wherein the construct further comprises an scFv or single-domain antibody (sdAb) covalently linked to the C-terminus of one of the two heavy chains of the IgG molecule or modified IgG molecule, the scFv or single-domain antibody (sdAb) containing one of said antigen binding sites for CD40.
5. The construct of item 4, wherein the construct further comprises an scFv or single-domain antibody (sdAb) covalently linked to the C-terminus of the other of the two heavy chains of the IgG molecule or modified IgG molecule, the scFv or single-domain antibody (sdAb) containing one of said antigen binding sites for CD40.
6. The construct of item 1, wherein the construct comprises an Fc fragment of an IgG molecule, and wherein each of the two N-termini and/or each of the two C-termini of said Fc fragment is covalently linked to a polypeptide chain comprising 1, 2 or 3, preferably 3 single-domain antibodies (sdAbs), wherein each of the 1, 2 or 3, preferably 3 single-domain antibodies (sdAbs) comprises one of said antigen binding sites for CD40.
7. The construct of any one of the preceding items, wherein the construct comprises 6 antigen binding sites for CD40.
8. The construct of any one of the preceding items, wherein the construct comprises 12 antigen binding sites for CD40.
9. The construct according to any one of the preceding items, wherein any two of the antigen binding sites for CD40 are in parallel orientation.
10. The construct of any one of items 2 to 9, wherein the construct comprises a trimer of three of the IgG molecules or modified IgG molecules, formed by a trimerization domain which is fused to the C-terminus of each heavy chain of each of the IgG molecules or modified IgG molecules.
11. The construct according to any one of items 4 to 10, wherein the trimerization domain is the trimerization domain of tenascin-C.
12. The construct according to item 10, wherein the trimerization domain comprises the amino acid sequence of SEQ ID NO: 60 or a sequence at least 70% identical thereto, preferably at least 80% identical thereto, more preferably at least 85% identical thereto, more preferably at least 90% identical thereto, more preferably at least 93% identical thereto, and more preferably at least 96% identical thereto.
13. The construct according to any one of items 2-12, wherein the IgG molecule or modified IgG molecule contains an oligomerization mutation.
14. The construct according to item 13, wherein the oligomerization mutation is an E345R/E430G/S440Y mutation.
15. The construct according to any one of the preceding items, wherein the construct comprises one or more Fab1 domains, scFv domains, and/or sdAb domains, each containing one antigen binding site for CD40.
16. The construct of any one of items 2-15, wherein the IgG molecule or modified IgG molecule is IgG1, IgG2, IgG3 or IgG4.
17. The construct of any one of items 2-16, wherein the IgG molecule or modified IgG molecule is IgG2 or IgG4.
18. The construct according to any one of items 2-17, wherein the Fc domain of the IgG molecule or modified IgG molecule or the Fc fragment of the IgG molecule contains a mutation reducing FcγR binding.
19. The construct according to item 18, wherein the mutation is an N297A mutation.
20. A multivalent anti-CD40 antibody construct comprising at least 2 antigen binding sites for CD40, wherein the antibody construct is not a natural IgA or IgM molecule, and wherein two of the antigen binding sites for CD40 are in antiparallel orientation.
21. The construct of item 20, wherein the domains containing the binding sites are linked by a linker sequence, preferably a peptide linker sequence.
22. The construct according to any one of the preceding items, wherein the construct is a CD40 agonist in an Fc-independent manner.
23. The construct according to any one of the preceding items, wherein the construct comprises:
   a. an amino acid sequence at least 85% identical to the amino acid sequence of SEQ ID NO: 1 and an amino acid sequence at least 85% identical to the amino acid sequence of SEQ ID NO: 2, preferably an amino acid sequence at least 90% identical to the amino acid sequence of SEQ ID NO: 1 and an amino acid sequence at least 90% identical to the amino acid sequence of SEQ ID NO: 2, more preferably an amino acid sequence at least 95% identical to the amino acid sequence of SEQ ID NO: 1 and an amino acid sequence at least 95% identical to the amino acid sequence of SEQ ID NO: 2, more preferably an amino acid sequence at least 98% identical to the amino acid sequence of SEQ ID NO: 1 and an amino acid sequence at least 98% identical to the amino acid sequence of SEQ ID NO: 2, more preferably an amino acid sequence at least 99% identical to the amino acid sequence of SEQ ID NO:1 and an amino acid sequence at least 99% identical to the amino acid sequence of SEQ ID NO: 2, and most preferably the amino acid sequence of SEQ ID NO: 1 and the amino acid sequence of SEQ ID NO: 2;
   b. an amino acid sequence at least 85% identical to the amino acid sequence of SEQ ID NO: 1 and an amino acid sequence at least 85% identical to the amino acid sequence of SEQ ID NO: 3, preferably an amino acid sequence at least 90% identical to the amino acid sequence of SEQ ID NO: 1 and an amino acid sequence at least 90% identical to the amino acid sequence of SEQ ID NO: 3, more preferably an amino acid sequence at least 95% identical to the amino acid sequence of SEQ ID NO: 1 and an amino acid sequence at least 95% identical to the amino acid sequence of SEQ ID NO: 3, more preferably an amino acid sequence at least 98% identical to the amino acid sequence of SEQ ID NO: 1 and an amino acid sequence at least 98% identical to the amino acid sequence of SEQ ID NO: 3, more preferably an amino acid sequence at least 99% identical to the amino acid sequence of SEQ ID NO:1 and an amino acid sequence at least 99% identical to the amino acid sequence of SEQ ID NO: 3, and most preferably the amino acid sequence of SEQ ID NO: 1 and the amino acid sequence of SEQ ID NO: 3;
   c. an amino acid sequence at least 85% identical to the amino acid sequence of SEQ ID NO: 1 and an amino acid sequence at least 85% identical to the amino acid sequence of SEQ ID NO: 4, preferably an amino acid sequence at least 90% identical to the amino acid sequence of SEQ ID NO: 1 and an amino acid sequence at least 90% identical to the amino acid sequence of SEQ ID NO: 4, more preferably an amino acid sequence at least 95% identical to the amino acid sequence of SEQ ID NO: 1 and an amino acid sequence at least 95% identical to the amino acid sequence of SEQ ID NO: 4, more preferably an amino acid sequence at least 98% identical to the amino acid sequence of SEQ ID NO: 1 and an amino acid sequence at least 98% identical to the amino acid sequence of SEQ ID NO: 4, more preferably an amino acid sequence at least 99% identical to the amino acid sequence of SEQ ID NO:1 and an amino acid sequence at least 99% identical to the amino acid sequence of SEQ ID NO: 4, and most preferably the amino acid sequence of SEQ ID NO: 1 and the amino acid sequence of SEQ ID NO: 4;
   d. an amino acid sequence at least 85% identical to the amino acid sequence of SEQ ID NO: 5 and an amino acid sequence at least 85% identical to the amino acid sequence of SEQ ID NO: 6, preferably an amino acid sequence at least 90% identical to the amino acid sequence of SEQ ID NO: 5 and an amino acid sequence at least 90% identical to the amino acid sequence of SEQ ID NO: 6, more preferably an amino acid sequence at least 95% identical to the amino acid sequence of SEQ ID NO: 5 and an amino acid sequence at least 95% identical to the amino acid sequence of SEQ ID NO: 6, more preferably an amino acid sequence at least 98% identical to the amino acid sequence of SEQ ID NO: 5 and an amino acid sequence at least 98% identical to the amino acid sequence of SEQ ID NO: 6, more preferably an amino acid sequence at least 99% identical to the amino acid sequence of SEQ ID NO:5 and an amino acid sequence at least 99% identical to the amino acid sequence of SEQ ID NO: 6, and most preferably the amino acid sequence of SEQ ID NO: 5 and the amino acid sequence of SEQ ID NO: 6;
   e. an amino acid sequence at least 85% identical to the amino acid sequence of SEQ ID NO: 5 and an amino acid sequence at least 85% identical to the amino acid sequence of SEQ ID NO: 7, preferably an amino acid sequence at least 90% identical to the amino acid sequence of SEQ ID NO: 5 and an amino acid sequence at least 90% identical to the amino acid sequence of SEQ ID NO: 7, more preferably an amino acid sequence at least 95% identical to the amino acid sequence of SEQ ID NO: 5 and an amino acid sequence at least 95% identical to the amino acid sequence of SEQ ID NO: 7, more preferably an amino acid sequence at least 98% identical to the amino acid sequence of SEQ ID NO: 5 and an amino acid sequence at least 98% identical to the amino acid sequence of SEQ ID NO: 7, more preferably an amino acid sequence at least 99% identical to the amino acid sequence of SEQ ID NO:5 and an amino acid sequence at least 99% identical to the amino acid sequence of SEQ ID NO: 6, and most preferably the amino acid sequence of SEQ ID NO: 5 and the amino acid sequence of SEQ ID NO: 7;
   f. an amino acid sequence at least 85% identical to the amino acid sequence of SEQ ID NO: 5 and an amino acid sequence at least 85% identical to the amino acid sequence of SEQ ID NO: 8, preferably an amino acid sequence at least 90% identical to the amino acid sequence of SEQ ID NO: 5 and an amino acid sequence at least 90% identical to the amino acid sequence of SEQ ID NO: 8, more preferably an amino acid sequence at least 95% identical to the amino acid sequence of SEQ ID NO: 5 and an amino acid sequence at least 95% identical to the amino acid sequence of SEQ ID NO: 8, more preferably an amino acid sequence at least 98% identical to the amino acid sequence of SEQ ID NO: 5 and an amino acid sequence at least 98% identical to the amino acid sequence of SEQ ID NO: 8, more preferably an amino acid sequence at least 99% identical to the amino acid sequence of SEQ ID NO:5 and an amino acid sequence at least 99% identical to the amino acid sequence of SEQ ID NO: 8, and most preferably the amino acid sequence of SEQ ID NO: 5 and the amino acid sequence of SEQ ID NO: 8;
   g. an amino acid sequence at least 85% identical to the amino acid sequence of SEQ ID NO: 9 and an amino acid sequence at least 85% identical to the amino acid sequence of SEQ ID NO: 10, preferably an amino acid sequence at least 90% identical to the amino acid sequence of SEQ ID NO: 9 and an amino acid sequence at least 90% identical to the amino acid sequence of SEQ ID NO: 10, more preferably an amino acid sequence at least 95% identical to the amino acid sequence of SEQ ID NO: 9 and an amino acid sequence at least 95% identical to the amino acid sequence of SEQ ID NO: 10, more preferably an amino acid sequence at least 98% identical to the amino acid sequence of SEQ ID NO: 9 and an amino acid sequence at least 98% identical to the amino acid sequence of SEQ ID NO: 10, more preferably an amino acid sequence at least 99% identical to the amino acid sequence of SEQ ID NO: 9 and an amino acid sequence at least 99% identical to the amino acid sequence of SEQ ID NO: 10, and most preferably the amino acid sequence of SEQ ID NO: 9 and the amino acid sequence of SEQ ID NO: 10;
   h. an amino acid sequence at least 85% identical to the amino acid sequence of SEQ ID NO: 9 and an amino acid sequence at least 85% identical to the amino acid sequence of SEQ ID NO: 11, preferably an amino acid sequence at least 90% identical to the amino acid sequence of SEQ ID NO: 9 and an amino acid sequence at least 90% identical to the amino acid sequence of SEQ ID NO: 11, more preferably an amino acid sequence at least 95% identical to the amino acid sequence of SEQ ID NO: 9 and an amino acid sequence at least 95% identical to the amino acid sequence of SEQ ID NO: 11, more preferably an amino acid sequence at least 98% identical to the amino acid sequence of SEQ ID NO: 9 and an amino acid sequence at least 98% identical to the amino acid sequence of SEQ ID NO: 11, more preferably an amino acid sequence at least 99% identical to the amino acid sequence of SEQ ID NO: 9 and an amino acid sequence at least 99% identical to the amino acid sequence of SEQ ID NO: 11, and most preferably the amino acid sequence of SEQ ID NO: 9 and the amino acid sequence of SEQ ID NO: 11;
   i. an amino acid sequence at least 85% identical to the amino acid sequence of SEQ ID NO: 9 and an amino acid sequence at least 85% identical to the amino acid sequence of SEQ ID NO: 12, preferably an amino acid sequence at least 90% identical to the amino acid sequence of SEQ ID NO: 9 and an amino acid sequence at least 90% identical to the amino acid sequence of SEQ ID NO: 12, more preferably an amino acid sequence at least 95% identical to the amino acid sequence of SEQ ID NO: 9 and an amino acid sequence at least 95% identical to the amino acid sequence of SEQ ID NO: 12, more preferably an amino acid sequence at least 98% identical to the amino acid sequence of SEQ ID NO: 9 and an amino acid sequence at least 98% identical to the amino acid sequence of SEQ ID NO: 12, more preferably an amino acid sequence at least 99% identical to the amino acid sequence of SEQ ID NO: 9 and an amino acid sequence at least 99% identical to the amino acid sequence of SEQ ID NO: 12, and most preferably the amino acid sequence of SEQ ID NO: 9 and the amino acid sequence of SEQ ID NO: 12;
   j. an amino acid sequence at least 85% identical to the amino acid sequence of SEQ ID NO: 13 and an amino acid sequence at least 85% identical to the amino acid sequence of SEQ ID NO: 14, preferably an amino acid sequence at least 90% identical to the amino acid sequence of SEQ ID NO: 13 and an amino acid sequence at least 90% identical to the amino acid sequence of SEQ ID NO: 14, more preferably an amino acid sequence at least 95% identical to the amino acid sequence of SEQ ID NO: 13 and an amino acid sequence at least 95% identical to the amino acid sequence of SEQ ID NO: 14, more preferably an amino acid sequence at least 98% identical to the amino acid sequence of SEQ ID NO: 13 and an amino acid sequence at least 98% identical to the amino acid sequence of SEQ ID NO: 14, more preferably an amino acid sequence at least 99% identical to the amino acid sequence of SEQ ID NO: 13 and an amino acid sequence at least 99% identical to the amino acid sequence of SEQ ID NO: 14, and most preferably the amino acid sequence of SEQ ID NO: 13 and the amino acid sequence of SEQ ID NO: 14;
   k. an amino acid sequence at least 85% identical to the amino acid sequence of SEQ ID NO: 13 and an amino acid sequence at least 85% identical to the amino acid sequence of SEQ ID NO: 15, preferably an amino acid sequence at least 90% identical to the amino acid sequence of SEQ ID NO: 13 and an amino acid sequence at least 90% identical to the amino acid sequence of SEQ ID NO: 15, more preferably an amino acid sequence at least 95% identical to the amino acid sequence of SEQ ID NO: 13 and an amino acid sequence at least 95% identical to the amino acid sequence of SEQ ID NO: 15, more preferably an amino acid sequence at least 98% identical to the amino acid sequence of SEQ ID NO: 13 and an amino acid sequence at least 98% identical to the amino acid sequence of SEQ ID NO: 15, more preferably an amino acid sequence at least 99% identical to the amino acid sequence of SEQ ID NO: 13 and an amino acid sequence at least 99% identical to the amino acid sequence of SEQ ID NO: 15, and most preferably the amino acid sequence of SEQ ID NO: 13 and the amino acid sequence of SEQ ID NO: 15;
   l. an amino acid sequence at least 85% identical to the amino acid sequence of SEQ ID NO: 13 and an amino acid sequence at least 85% identical to the amino acid sequence of SEQ ID NO: 16, preferably an amino acid sequence at least 90% identical to the amino acid sequence of SEQ ID NO: 13 and an amino acid sequence at least 90% identical to the amino acid sequence of SEQ ID NO: 16, more preferably an amino acid sequence at least 95% identical to the amino acid sequence of SEQ ID NO: 13 and an amino acid sequence at least 95% identical to the amino acid sequence of SEQ ID NO: 16, more preferably an amino acid sequence at least 98% identical to the amino acid sequence of SEQ ID NO: 13 and an amino acid sequence at least 98% identical to the amino acid sequence of SEQ ID NO: 16, more preferably an amino acid sequence at least 99% identical to the amino acid sequence of SEQ ID NO: 13 and an amino acid sequence at least 99% identical to the amino acid sequence of SEQ ID NO: 16, and most preferably the amino acid sequence of SEQ ID NO: 13 and the amino acid sequence of SEQ ID NO: 16;
   m. an amino acid sequence at least 85% identical to the amino acid sequence of SEQ ID NO: 17 and an amino acid sequence at least 85% identical to the amino acid sequence of SEQ ID NO: 18, preferably an amino acid sequence at least 90% identical to the amino acid sequence of SEQ ID NO: 17 and an amino acid sequence at least 90% identical to the amino acid sequence of SEQ ID NO: 18, more preferably an amino acid sequence at least 95% identical to the amino acid sequence of SEQ ID NO: 17 and an amino acid sequence at least 95% identical to the amino acid sequence of SEQ ID NO: 18, more preferably an amino acid sequence at least 98% identical to the amino acid sequence of SEQ ID NO: 17 and an amino acid sequence at least 98% identical to the amino acid sequence of SEQ ID NO: 18, more preferably an amino acid sequence at least 99% identical to the amino acid sequence of SEQ ID NO: 17 and an amino acid sequence at least 99% identical to the amino acid sequence of SEQ ID NO: 18, and most preferably the amino acid sequence of SEQ ID NO: 17 and the amino acid sequence of SEQ ID NO: 18;
   n. an amino acid sequence at least 85% identical to the amino acid sequence of SEQ ID NO: 17 and an amino acid sequence at least 85% identical to the amino acid sequence of SEQ ID NO: 19, preferably an amino acid sequence at least 90% identical to the amino acid sequence of SEQ ID NO: 17 and an amino acid sequence at least 90% identical to the amino acid sequence of SEQ ID NO: 19, more preferably an amino acid sequence at least 95% identical to the amino acid sequence of SEQ ID NO: 17 and an amino acid sequence at least 95% identical to the amino acid sequence of SEQ ID NO: 19, more preferably an amino acid sequence at least 98% identical to the amino acid sequence of SEQ ID NO: 17 and an amino acid sequence at least 98% identical to the amino acid sequence of SEQ ID NO: 19, more preferably an amino acid sequence at least 99% identical to the amino acid sequence of SEQ ID NO: 17 and an amino acid sequence at least 99% identical to the amino acid sequence of SEQ ID NO: 19, and most preferably the amino acid sequence of SEQ ID NO: 17 and the amino acid sequence of SEQ ID NO: 19;
   o. an amino acid sequence at least 85% identical to the amino acid sequence of SEQ ID NO: 17 and an amino acid sequence at least 85% identical to the amino acid sequence of SEQ ID NO: 20, preferably an amino acid sequence at least 90% identical to the amino acid sequence of SEQ ID NO: 17 and an amino acid sequence at least 90% identical to the amino acid sequence of SEQ ID NO: 20, more preferably an amino acid sequence at least 95% identical to the amino acid sequence of SEQ ID NO: 17 and an amino acid sequence at least 95% identical to the amino acid sequence of SEQ ID NO: 20, more preferably an amino acid sequence at least 98% identical to the amino acid sequence of SEQ ID NO: 17 and an amino acid sequence at least 98% identical to the amino acid sequence of SEQ ID NO: 20, more preferably an amino acid sequence at least 99% identical to the amino acid sequence of SEQ ID NO: 17 and an amino acid sequence at least 99% identical to the amino acid sequence of SEQ ID NO: 20, and most preferably the amino acid sequence of SEQ ID NO: 17 and the amino acid sequence of SEQ ID NO: 20;
   p. an amino acid sequence at least 85% identical to the amino acid sequence of SEQ ID NO: 21 and an amino acid sequence at least 85% identical to the amino acid sequence of SEQ ID NO: 24, preferably an amino acid sequence at least 90% identical to the amino acid sequence of SEQ ID NO: 21 and an amino acid sequence at least 90% identical to the amino acid sequence of SEQ ID NO: 24, more preferably an amino acid sequence at least 95% identical to the amino acid sequence of SEQ ID NO: 21 and an amino acid sequence at least 95% identical to the amino acid sequence of SEQ ID NO: 24, more preferably an amino acid sequence at least 98% identical to the amino acid sequence of SEQ ID NO: 21 and an amino acid sequence at least 98% identical to the amino acid sequence of SEQ ID NO: 24, more preferably an amino acid sequence at least 99% identical to the amino acid sequence of SEQ ID NO: 21 and an amino acid sequence at least 99% identical to the amino acid sequence of SEQ ID NO: 24, and most preferably the amino acid sequence of SEQ ID NO: 21 and the amino acid sequence of SEQ ID NO: 24;
   q. an amino acid sequence at least 85% identical to the amino acid sequence of SEQ ID NO: 21 and an amino acid sequence at least 85% identical to the amino acid sequence of SEQ ID NO: 25, preferably an amino acid sequence at least 90% identical to the amino acid sequence of SEQ ID NO: 21 and an amino acid sequence at least 90% identical to the amino acid sequence of SEQ ID NO: 25, more preferably an amino acid sequence at least 95% identical to the amino acid sequence of SEQ ID NO: 21 and an amino acid sequence at least 95% identical to the amino acid sequence of SEQ ID NO: 25, more preferably an amino acid sequence at least 98% identical to the amino acid sequence of SEQ ID NO: 21 and an amino acid sequence at least 98% identical to the amino acid sequence of SEQ ID NO: 25, more preferably an amino acid sequence at least 99% identical to the amino acid sequence of SEQ ID NO: 21 and an amino acid sequence at least 99% identical to the amino acid sequence of SEQ ID NO: 25, and most preferably the amino acid sequence of SEQ ID NO: 21 and the amino acid sequence of SEQ ID NO: 25;
   r. an amino acid sequence at least 85% identical to the amino acid sequence of SEQ ID NO: 21 and an amino acid sequence at least 85% identical to the amino acid sequence of SEQ ID NO: 26, preferably an amino acid sequence at least 90% identical to the amino acid sequence of SEQ ID NO: 21 and an amino acid sequence at least 90% identical to the amino acid sequence of SEQ ID NO: 26, more preferably an amino acid sequence at least 95% identical to the amino acid sequence of SEQ ID NO: 21 and an amino acid sequence at least 95% identical to the amino acid sequence of SEQ ID NO: 26, more preferably an amino acid sequence at least 98% identical to the amino acid sequence of SEQ ID NO: 21 and an amino acid sequence at least 98% identical to the amino acid sequence of SEQ ID NO: 26, more preferably an amino acid sequence at least 99% identical to the amino acid sequence of SEQ ID NO: 21 and an amino acid sequence at least 99% identical to the amino acid sequence of SEQ ID NO: 26, and most preferably the amino acid sequence of SEQ ID NO: 21 and the amino acid sequence of SEQ ID NO: 26;
   s. an amino acid sequence at least 85% identical to the amino acid sequence of SEQ ID NO: 22 and an amino acid sequence at least 85% identical to the amino acid sequence of SEQ ID NO: 24, preferably an amino acid sequence at least 90% identical to the amino acid sequence of SEQ ID NO: 22 and an amino acid sequence at least 90% identical to the amino acid sequence of SEQ ID NO: 24, more preferably an amino acid sequence at least 95% identical to the amino acid sequence of SEQ ID NO: 22 and an amino acid sequence at least 95% identical to the amino acid sequence of SEQ ID NO: 24, more preferably an amino acid sequence at least 98% identical to the amino acid sequence of SEQ ID NO: 22 and an amino acid sequence at least 98% identical to the amino acid sequence of SEQ ID NO: 24, more preferably an amino acid sequence at least 99% identical to the amino acid sequence of SEQ ID NO: 22 and an amino acid sequence at least 99% identical to the amino acid sequence of SEQ ID NO: 24, and most preferably the amino acid sequence of SEQ ID NO: 22 and the amino acid sequence of SEQ ID NO: 24;
   t. an amino acid sequence at least 85% identical to the amino acid sequence of SEQ ID NO: 22 and an amino acid sequence at least 85% identical to the amino acid sequence of SEQ ID NO: 25, preferably an amino acid sequence at least 90% identical to the amino acid sequence of SEQ ID NO: 22 and an amino acid sequence at least 90% identical to the amino acid sequence of SEQ ID NO: 25, more preferably an amino acid sequence at least 95% identical to the amino acid sequence of SEQ ID NO: 22 and an amino acid sequence at least 95% identical to the amino acid sequence of SEQ ID NO: 25, more preferably an amino acid sequence at least 98% identical to the amino acid sequence of SEQ ID NO: 22 and an amino acid sequence at least 98% identical to the amino acid sequence of SEQ ID NO: 25, more preferably an amino acid sequence at least 99% identical to the amino acid sequence of SEQ ID NO: 22 and an amino acid sequence at least 99% identical to the amino acid sequence of SEQ ID NO: 25, and most preferably the amino acid sequence of SEQ ID NO: 22 and the amino acid sequence of SEQ ID NO: 25;
   u. an amino acid sequence at least 85% identical to the amino acid sequence of SEQ ID NO: 22 and an amino acid sequence at least 85% identical to the amino acid sequence of SEQ ID NO: 26, preferably an amino acid sequence at least 90% identical to the amino acid sequence of SEQ ID NO: 22 and an amino acid sequence at least 90% identical to the amino acid sequence of SEQ ID NO: 26, more preferably an amino acid sequence at least 95% identical to the amino acid sequence of SEQ ID NO: 22 and an amino acid sequence at least 95% identical to the amino acid sequence of SEQ ID NO: 26, more preferably an amino acid sequence at least 98% identical to the amino acid sequence of SEQ ID NO: 22 and an amino acid sequence at least 98% identical to the amino acid sequence of SEQ ID NO: 26, more preferably an amino acid sequence at least 99% identical to the amino acid sequence of SEQ ID NO: 22 and an amino acid sequence at least 99% identical to the amino acid sequence of SEQ ID NO: 26, and most preferably the amino acid sequence of SEQ ID NO: 22 and the amino acid sequence of SEQ ID NO: 26;
   v. an amino acid sequence at least 85% identical to the amino acid sequence of SEQ ID NO: 23 and an amino acid sequence at least 85% identical to the amino acid sequence of SEQ ID NO: 24, preferably an amino acid sequence at least 90% identical to the amino acid sequence of SEQ ID NO: 23 and an amino acid sequence at least 90% identical to the amino acid sequence of SEQ ID NO: 24, more preferably an amino acid sequence at least 95% identical to the amino acid sequence of SEQ ID NO: 23 and an amino acid sequence at least 95% identical to the amino acid sequence of SEQ ID NO: 24, more preferably an amino acid sequence at least 98% identical to the amino acid sequence of SEQ ID NO: 23 and an amino acid sequence at least 98% identical to the amino acid sequence of SEQ ID NO: 24, more preferably an amino acid sequence at least 99% identical to the amino acid sequence of SEQ ID NO: 23 and an amino acid sequence at least 99% identical to the amino acid sequence of SEQ ID NO: 24, and most preferably the amino acid sequence of SEQ ID NO: 23 and the amino acid sequence of SEQ ID NO: 24;
   w. an amino acid sequence at least 85% identical to the amino acid sequence of SEQ ID NO: 23 and an amino acid sequence at least 85% identical to the amino acid sequence of SEQ ID NO: 25, preferably an amino acid sequence at least 90% identical to the amino acid sequence of SEQ ID NO: 23 and an amino acid sequence at least 90% identical to the amino acid sequence of SEQ ID NO: 25, more preferably an amino acid sequence at least 95% identical to the amino acid sequence of SEQ ID NO: 23 and an amino acid sequence at least 95% identical to the amino acid sequence of SEQ ID NO: 25, more preferably an amino acid sequence at least 98% identical to the amino acid sequence of SEQ ID NO: 23 and an amino acid sequence at least 98% identical to the amino acid sequence of SEQ ID NO: 25, more preferably an amino acid sequence at least 99% identical to the amino acid sequence of SEQ ID NO: 23 and an amino acid sequence at least 99% identical to the amino acid sequence of SEQ ID NO: 25, and most preferably the amino acid sequence of SEQ ID NO: 23 and the amino acid sequence of SEQ ID NO: 25;
   x. an amino acid sequence at least 85% identical to the amino acid sequence of SEQ ID NO: 23 and an amino acid sequence at least 85% identical to the amino acid sequence of SEQ ID NO: 26, preferably an amino acid sequence at least 90% identical to the amino acid sequence of SEQ ID NO: 23 and an amino acid sequence at least 90% identical to the amino acid sequence of SEQ ID NO: 26, more preferably an amino acid sequence at least 95% identical to the amino acid sequence of SEQ ID NO: 23 and an amino acid sequence at least 95% identical to the amino acid sequence of SEQ ID NO: 26, more preferably an amino acid sequence at least 98% identical to the amino acid sequence of SEQ ID NO: 23 and an amino acid sequence at least 98% identical to the amino acid sequence of SEQ ID NO: 26, more preferably an amino acid sequence at least 99% identical to the amino acid sequence of SEQ ID NO: 23 and an amino acid sequence at least 99% identical to the amino acid sequence of SEQ ID NO: 26, and most preferably the amino acid sequence of SEQ ID NO: 23 and the amino acid sequence of SEQ ID NO: 26;
   y. an amino acid sequence at least 85% identical to the amino acid sequence of SEQ ID NO: 27, preferably an amino acid sequence at least 90% identical to the amino acid sequence of SEQ ID NO: 27, more preferably an amino acid sequence at least 95% identical to the amino acid sequence of SEQ ID NO: 27, more preferably an amino acid sequence at least 98% identical to the amino acid sequence of SEQ ID NO: 27, more preferably an amino acid sequence at least 99% identical to the amino acid sequence of SEQ ID NO: 27, and most preferably the amino acid sequence of SEQ ID NO: 27;
   z. an amino acid sequence at least 85% identical to the amino acid sequence of SEQ ID NO: 28, preferably an amino acid sequence at least 90% identical to the amino acid sequence of SEQ ID NO: 28, more preferably an amino acid sequence at least 95% identical to the amino acid sequence of SEQ ID NO: 28, more preferably an amino acid sequence at least 98% identical to the amino acid sequence of SEQ ID NO: 28, more preferably an amino acid sequence at least 99% identical to the amino acid sequence of SEQ ID NO: 28, and most preferably the amino acid sequence of SEQ ID NO: 28;
   aa. an amino acid sequence at least 85% identical to the amino acid sequence of SEQ ID NO: 29, preferably an amino acid sequence at least 90% identical to the amino acid sequence of SEQ ID NO: 29, more preferably an amino acid sequence at least 95% identical to the amino acid sequence of SEQ ID NO: 29, more preferably an amino acid sequence at least 98% identical to the amino acid sequence of SEQ ID NO: 29, more preferably an amino acid sequence at least 99% identical to the amino acid sequence of SEQ ID NO: 29, and most preferably the amino acid sequence of SEQ ID NO: 29;
   bb. an amino acid sequence at least 85% identical to the amino acid sequence of SEQ ID NO: 31, preferably an amino acid sequence at least 90% identical to the amino acid sequence of SEQ ID NO: 31, more preferably an amino acid sequence at least 95% identical to the amino acid sequence of SEQ ID NO: 31, more preferably an amino acid sequence at least 98% identical to the amino acid sequence of SEQ ID NO: 31, more preferably an amino acid sequence at least 99% identical to the amino acid sequence of SEQ ID NO: 31, and most preferably the amino acid sequence of SEQ ID NO: 31;
   cc. an amino acid sequence at least 85% identical to the amino acid sequence of SEQ ID NO: 32, preferably an amino acid sequence at least 90% identical to the amino acid sequence of SEQ ID NO: 32, more preferably an amino acid sequence at least 95% identical to the amino acid sequence of SEQ ID NO: 32, more preferably an amino acid sequence at least 98% identical to the amino acid sequence of SEQ ID NO: 32, more preferably an amino acid sequence at least 99% identical to the amino acid sequence of SEQ ID NO: 32, and most preferably the amino acid sequence of SEQ ID NO: 32;
   dd. an amino acid sequence at least 85% identical to the amino acid sequence of SEQ ID NO: 33, preferably an amino acid sequence at least 90% identical to the amino acid sequence of SEQ ID NO: 33, more preferably an amino acid sequence at least 95% identical to the amino acid sequence of SEQ ID NO: 33, more preferably an amino acid sequence at least 98% identical to the amino acid sequence of SEQ ID NO: 33, more preferably an amino acid sequence at least 99% identical to the amino acid sequence of SEQ ID NO: 33, and most preferably the amino acid sequence of SEQ ID NO: 33;
   ee. an amino acid sequence at least 85% identical to the amino acid sequence of SEQ ID NO: 34, preferably an amino acid sequence at least 90% identical to the amino acid sequence of SEQ ID NO: 34, more preferably an amino acid sequence at least 95% identical to the amino acid sequence of SEQ ID NO: 34, more preferably an amino acid sequence at least 98% identical to the amino acid sequence of SEQ ID NO: 34, more preferably an amino acid sequence at least 99% identical to the amino acid sequence of SEQ ID NO: 34, and most preferably the amino acid sequence of SEQ ID NO: 34;
   ff. an amino acid sequence at least 85% identical to the amino acid sequence of SEQ ID NO: 35, preferably an amino acid sequence at least 90% identical to the amino acid sequence of SEQ ID NO: 35, more preferably an amino acid sequence at least 95% identical to the amino acid sequence of SEQ ID NO: 35, more preferably an amino acid sequence at least 98% identical to the amino acid sequence of SEQ ID NO: 35, more preferably an amino acid sequence at least 99% identical to the amino acid sequence of SEQ ID NO: 35, and most preferably the amino acid sequence of SEQ ID NO: 35;
   gg. an amino acid sequence at least 85% identical to the amino acid sequence of SEQ ID NO: 21 and an amino acid sequence at least 85% identical to the amino acid sequence of SEQ ID NO: 36, preferably an amino acid sequence at least 90% identical to the amino acid sequence of SEQ ID NO: 21 and an amino acid sequence at least 90% identical to the amino acid sequence of SEQ ID NO: 36, more preferably an amino acid sequence at least 95% identical to the amino acid sequence of SEQ ID NO: 21 and an amino acid sequence at least 95% identical to the amino acid sequence of SEQ ID NO: 36, more preferably an amino acid sequence at least 98% identical to the amino acid sequence of SEQ ID NO: 21 and an amino acid sequence at least 98% identical to the amino acid sequence of SEQ ID NO: 36, more preferably an amino acid sequence at least 99% identical to the amino acid sequence of SEQ ID NO: 21 and an amino acid sequence at least 99% identical to the amino acid sequence of SEQ ID NO: 36, and most preferably the amino acid sequence of SEQ ID NO: 21 and the amino acid sequence of SEQ ID NO: 36;
   hh. an amino acid sequence at least 85% identical to the amino acid sequence of SEQ ID NO: 22 and an amino acid sequence at least 85% identical to the amino acid sequence of SEQ ID NO: 36, preferably an amino acid sequence at least 90% identical to the amino acid sequence of SEQ ID NO: 22 and an amino acid sequence at least 90% identical to the amino acid sequence of SEQ ID NO: 36, more preferably an amino acid sequence at least 95% identical to the amino acid sequence of SEQ ID NO: 22 and an amino acid sequence at least 95% identical to the amino acid sequence of SEQ ID NO: 36, more preferably an amino acid sequence at least 98% identical to the amino acid sequence of SEQ ID NO: 22 and an amino acid sequence at least 98% identical to the amino acid sequence of SEQ ID NO: 36, more preferably an amino acid sequence at least 99% identical to the amino acid sequence of SEQ ID NO: 22 and an amino acid sequence at least 99% identical to the amino acid sequence of SEQ ID NO: 36, and most preferably the amino acid sequence of SEQ ID NO: 22 and the amino acid sequence of SEQ ID NO: 36;
   ii. an amino acid sequence at least 85% identical to the amino acid sequence of SEQ ID NO: 23 and an amino acid sequence at least 85% identical to the amino acid sequence of SEQ ID NO: 36, preferably an amino acid sequence at least 90% identical to the amino acid sequence of SEQ ID NO: 23 and an amino acid sequence at least 90% identical to the amino acid sequence of SEQ ID NO: 36, more preferably an amino acid sequence at least 95% identical to the amino acid sequence of SEQ ID NO: 23 and an amino acid sequence at least 95% identical to the amino acid sequence of SEQ ID NO: 36, more preferably an amino acid sequence at least 98% identical to the amino acid sequence of SEQ ID NO: 23 and an amino acid sequence at least 98% identical to the amino acid sequence of SEQ ID NO: 36, more preferably an amino acid sequence at least 99% identical to the amino acid sequence of SEQ ID NO: 23 and an amino acid sequence at least 99% identical to the amino acid sequence of SEQ ID NO: 36, and most preferably the amino acid sequence of SEQ ID NO: 23 and the amino acid sequence of SEQ ID NO: 36;
   jj. an amino acid sequence at least 85% identical to the amino acid sequence of SEQ ID NO: 21 and an amino acid sequence at least 85% identical to the amino acid sequence of SEQ ID NO: 39, preferably an amino acid sequence at least 90% identical to the amino acid sequence of SEQ ID NO: 21 and an amino acid sequence at least 90% identical to the amino acid sequence of SEQ ID NO: 39, more preferably an amino acid sequence at least 95% identical to the amino acid sequence of SEQ ID NO: 21 and an amino acid sequence at least 95% identical to the amino acid sequence of SEQ ID NO: 39, more preferably an amino acid sequence at least 98% identical to the amino acid sequence of SEQ ID NO: 21 and an amino acid sequence at least 98% identical to the amino acid sequence of SEQ ID NO: 39, more preferably an amino acid sequence at least 99% identical to the amino acid sequence of SEQ ID NO: 21 and an amino acid sequence at least 99% identical to the amino acid sequence of SEQ ID NO: 39, and most preferably the amino acid sequence of SEQ ID NO: 21 and the amino acid sequence of SEQ ID NO: 39;
   kk. an amino acid sequence at least 85% identical to the amino acid sequence of SEQ ID NO: 22 and an amino acid sequence at least 85% identical to the amino acid sequence of SEQ ID NO: 39, preferably an amino acid sequence at least 90% identical to the amino acid sequence of SEQ ID NO: 22 and an amino acid sequence at least 90% identical to the amino acid sequence of SEQ ID NO: 39, more preferably an amino acid sequence at least 95% identical to the amino acid sequence of SEQ ID NO: 22 and an amino acid sequence at least 95% identical to the amino acid sequence of SEQ ID NO: 39, more preferably an amino acid sequence at least 98% identical to the amino acid sequence of SEQ ID NO: 22 and an amino acid sequence at least 98% identical to the amino acid sequence of SEQ ID NO: 39, more preferably an amino acid sequence at least 99% identical to the amino acid sequence of SEQ ID NO: 22 and an amino acid sequence at least 99% identical to the amino acid sequence of SEQ ID NO: 39, and most preferably the amino acid sequence of SEQ ID NO: 22 and the amino acid sequence of SEQ ID NO: 39;
   ll. an amino acid sequence at least 85% identical to the amino acid sequence of SEQ ID NO: 23 and an amino acid sequence at least 85% identical to the amino acid sequence of SEQ ID NO: 39, preferably an amino acid sequence at least 90% identical to the amino acid sequence of SEQ ID NO: 23 and an amino acid sequence at least 90% identical to the amino acid sequence of SEQ ID NO: 39, more preferably an amino acid sequence at least 95% identical to the amino acid sequence of SEQ ID NO: 23 and an amino acid sequence at least 95% identical to the amino acid sequence of SEQ ID NO: 39, more preferably an amino acid sequence at least 98% identical to the amino acid sequence of SEQ ID NO: 23 and an amino acid sequence at least 98% identical to the amino acid sequence of SEQ ID NO: 39, more preferably an amino acid sequence at least 99% identical to the amino acid sequence of SEQ ID NO: 23 and an amino acid sequence at least 99% identical to the amino acid sequence of SEQ ID NO: 39, and most preferably the amino acid sequence of SEQ ID NO: 23 and the amino acid sequence of SEQ ID NO: 39;
   mm. an amino acid sequence at least 85% identical to the amino acid sequence of SEQ ID NO: 21 and an amino acid sequence at least 85% identical to the amino acid sequence of SEQ ID NO: 40, preferably an amino acid sequence at least 90% identical to the amino acid sequence of SEQ ID NO: 21 and an amino acid sequence at least 90% identical to the amino acid sequence of SEQ ID NO: 40, more preferably an amino acid sequence at least 95% identical to the amino acid sequence of SEQ ID NO: 21 and an amino acid sequence at least 95% identical to the amino acid sequence of SEQ ID NO: 40, more preferably an amino acid sequence at least 98% identical to the amino acid sequence of SEQ ID NO: 21 and an amino acid sequence at least 98% identical to the amino acid sequence of SEQ ID NO: 40, more preferably an amino acid sequence at least 99% identical to the amino acid sequence of SEQ ID NO: 21 and an amino acid sequence at least 99% identical to the amino acid sequence of SEQ ID NO: 40, and most preferably the amino acid sequence of SEQ ID NO: 21 and the amino acid sequence of SEQ ID NO: 40;
   nn. an amino acid sequence at least 85% identical to the amino acid sequence of SEQ ID NO: 22 and an amino acid sequence at least 85% identical to the amino acid sequence of SEQ ID NO: 40, preferably an amino acid sequence at least 90% identical to the amino acid sequence of SEQ ID NO: 22 and an amino acid sequence at least 90% identical to the amino acid sequence of SEQ ID NO: 40, more preferably an amino acid sequence at least 95% identical to the amino acid sequence of SEQ ID NO: 22 and an amino acid sequence at least 95% identical to the amino acid sequence of SEQ ID NO: 40, more preferably an amino acid sequence at least 98% identical to the amino acid sequence of SEQ ID NO: 22 and an amino acid sequence at least 98% identical to the amino acid sequence of SEQ ID NO: 40, more preferably an amino acid sequence at least 99% identical to the amino acid sequence of SEQ ID NO: 22 and an amino acid sequence at least 99% identical to the amino acid sequence of SEQ ID NO: 40, and most preferably the amino acid sequence of SEQ ID NO: 22 and the amino acid sequence of SEQ ID NO: 40;
   oo. an amino acid sequence at least 85% identical to the amino acid sequence of SEQ ID NO: 23 and an amino acid sequence at least 85% identical to the amino acid sequence of SEQ ID NO: 40, preferably an amino acid sequence at least 90% identical to the amino acid sequence of SEQ ID NO: 23 and an amino acid sequence at least 90% identical to the amino acid sequence of SEQ ID NO: 40, more preferably an amino acid sequence at least 95% identical to the amino acid sequence of SEQ ID NO: 23 and an amino acid sequence at least 95% identical to the amino acid sequence of SEQ ID NO: 40, more preferably an amino acid sequence at least 98% identical to the amino acid sequence of SEQ ID NO: 23 and an amino acid sequence at least 98% identical to the amino acid sequence of SEQ ID NO: 40, more preferably an amino acid sequence at least 99% identical to the amino acid sequence of SEQ ID NO: 23 and an amino acid sequence at least 99% identical to the amino acid sequence of SEQ ID NO: 40, and most preferably the amino acid sequence of SEQ ID NO: 23 and the amino acid sequence of SEQ ID NO: 40;
   pp. an amino acid sequence at least 85% identical to the amino acid sequence of SEQ ID NO: 21 and an amino acid sequence at least 85% identical to the amino acid sequence of SEQ ID NO: 41, preferably an amino acid sequence at least 90% identical to the amino acid sequence of SEQ ID NO: 21 and an amino acid sequence at least 90% identical to the amino acid sequence of SEQ ID NO: 41, more preferably an amino acid sequence at least 95% identical to the amino acid sequence of SEQ ID NO: 21 and an amino acid sequence at least 95% identical to the amino acid sequence of SEQ ID NO: 41, more preferably an amino acid sequence at least 98% identical to the amino acid sequence of SEQ ID NO: 21 and an amino acid sequence at least 98% identical to the amino acid sequence of SEQ ID NO: 41, more preferably an amino acid sequence at least 99% identical to the amino acid sequence of SEQ ID NO: 21 and an amino acid sequence at least 99% identical to the amino acid sequence of SEQ ID NO: 41, and most preferably the amino acid sequence of SEQ ID NO: 21 and the amino acid sequence of SEQ ID NO: 41;
   qq. an amino acid sequence at least 85% identical to the amino acid sequence of SEQ ID NO: 22 and an amino acid sequence at least 85% identical to the amino acid sequence of SEQ ID NO: 41, preferably an amino acid sequence at least 90% identical to the amino acid sequence of SEQ ID NO: 22 and an amino acid sequence at least 90% identical to the amino acid sequence of SEQ ID NO: 41, more preferably an amino acid sequence at least 95% identical to the amino acid sequence of SEQ ID NO: 22 and an amino acid sequence at least 95% identical to the amino acid sequence of SEQ ID NO: 41, more preferably an amino acid sequence at least 98% identical to the amino acid sequence of SEQ ID NO: 22 and an amino acid sequence at least 98% identical to the amino acid sequence of SEQ ID NO: 41, more preferably an amino acid sequence at least 99% identical to the amino acid sequence of SEQ ID NO: 22 and an amino acid sequence at least 99% identical to the amino acid sequence of SEQ ID NO: 41, and most preferably the amino acid sequence of SEQ ID NO: 22 and the amino acid sequence of SEQ ID NO: 41;
   rr. an amino acid sequence at least 85% identical to the amino acid sequence of SEQ ID NO: 23 and an amino acid sequence at least 85% identical to the amino acid sequence of SEQ ID NO: 41, preferably an amino acid sequence at least 90% identical to the amino acid sequence of SEQ ID NO: 23 and an amino acid sequence at least 90% identical to the amino acid sequence of SEQ ID NO: 41, more preferably an amino acid sequence at least 95% identical to the amino acid sequence of SEQ ID NO: 23 and an amino acid sequence at least 95% identical to the amino acid sequence of SEQ ID NO: 41, more preferably an amino acid sequence at least 98% identical to the amino acid sequence of SEQ ID NO: 23 and an amino acid sequence at least 98% identical to the amino acid sequence of SEQ ID NO: 41, more preferably an amino acid sequence at least 99% identical to the amino acid sequence of SEQ ID NO: 23 and an amino acid sequence at least 99% identical to the amino acid sequence of SEQ ID NO: 41, and most preferably the amino acid sequence of SEQ ID NO: 23 and the amino acid sequence of SEQ ID NO: 41;
   ss. an amino acid sequence at least 85% identical to the amino acid sequence of SEQ ID NO: 42, preferably an amino acid sequence at least 90% identical to the amino acid sequence of SEQ ID NO: 42, more preferably an amino acid sequence at least 95% identical to the amino acid sequence of SEQ ID NO: 42, more preferably an amino acid sequence at least 98% identical to the amino acid sequence of SEQ ID NO: 42, more preferably an amino acid sequence at least 99% identical to the amino acid sequence of SEQ ID NO: 42, and most preferably the amino acid sequence of SEQ ID NO: 42;
   tt. an amino acid sequence of a single-domain antibody (sdAb) and an amino acid sequence at least 85% identical to the amino acid sequence of SEQ ID NO: 39, preferably an amino acid sequence of a single-domain antibody (sdAb) and an amino acid sequence at least 90% identical to the amino acid sequence of SEQ ID NO: 39, more preferably an amino acid sequence of a single-domain antibody (sdAb) and an amino acid sequence at least 95% identical to the amino acid sequence of SEQ ID NO: 39, more preferably an amino acid sequence of a single-domain antibody (sdAb) and an amino acid sequence at least 98% identical to the amino acid sequence of SEQ ID NO: 39, more preferably an amino acid sequence of a single-domain antibody (sdAb) and an amino acid sequence at least 99% identical to the amino acid sequence of SEQ ID NO: 39, and most preferably an amino acid sequence of a single-domain antibody (sdAb) and the amino acid sequence of SEQ ID NO: 39;
   uu. an amino acid sequence of a single-domain antibody (sdAb) and an amino acid sequence at least 85% identical to the amino acid sequence of SEQ ID NO: 41, preferably an amino acid sequence of a single-domain antibody (sdAb) and an amino acid sequence at least 90% identical to the amino acid sequence of SEQ ID NO: 41, more preferably an amino acid sequence of a single-domain antibody (sdAb) and an amino acid sequence at least 95% identical to the amino acid sequence of SEQ ID NO: 41, more preferably an amino acid sequence of a single-domain antibody (sdAb) and an amino acid sequence at least 98% identical to the amino acid sequence of SEQ ID NO: 41, more preferably an amino acid sequence of a single-domain antibody (sdAb) and an amino acid sequence at least 99% identical to the amino acid sequence of SEQ ID NO: 41, and most preferably an amino acid sequence of a single-domain antibody (sdAb) and the amino acid sequence of SEQ ID NO: 41;
   w. an amino acid sequence at least 85% identical to the amino acid sequence of SEQ ID NO: 43 and an amino acid sequence at least 85% identical to the amino acid sequence of SEQ ID NO: 44, preferably an amino acid sequence at least 90% identical to the amino acid sequence of SEQ ID NO: 43 and an amino acid sequence at least 90% identical to the amino acid sequence of SEQ ID NO: 44, more preferably an amino acid sequence at least 95% identical to the amino acid sequence of SEQ ID NO: 43 and an amino acid sequence at least 95% identical to the amino acid sequence of SEQ ID NO: 44, more preferably an amino acid sequence at least 98% identical to the amino acid sequence of SEQ ID NO: 43 and an amino acid sequence at least 98% identical to the amino acid sequence of SEQ ID NO: 44, more preferably an amino acid sequence at least 99% identical to the amino acid sequence of SEQ ID NO: 43 and an amino acid sequence at least 99% identical to the amino acid sequence of SEQ ID NO: 44, and most preferably the amino acid sequence of SEQ ID NO: 43 and the amino acid sequence of SEQ ID NO: 44;
   ww. an amino acid sequence at least 85% identical to the amino acid sequence of SEQ ID NO: 43 and an amino acid sequence at least 85% identical to the amino acid sequence of SEQ ID NO: 45, preferably an amino acid sequence at least 90% identical to the amino acid sequence of SEQ ID NO: 43 and an amino acid sequence at least 90% identical to the amino acid sequence of SEQ ID NO: 45, more preferably an amino acid sequence at least 95% identical to the amino acid sequence of SEQ ID NO: 43 and an amino acid sequence at least 95% identical to the amino acid sequence of SEQ ID NO: 45, more preferably an amino acid sequence at least 98% identical to the amino acid sequence of SEQ ID NO: 43 and an amino acid sequence at least 98% identical to the amino acid sequence of SEQ ID NO: 45, more preferably an amino acid sequence at least 99% identical to the amino acid sequence of SEQ ID NO: 43 and an amino acid sequence at least 99% identical to the amino acid sequence of SEQ ID NO: 45, and most preferably the amino acid sequence of SEQ ID NO: 43 and the amino acid sequence of SEQ ID NO: 45;
   xx. an amino acid sequence at least 85% identical to the amino acid sequence of SEQ ID NO: 43 and an amino acid sequence at least 85% identical to the amino acid sequence of SEQ ID NO: 46, preferably an amino acid sequence at least 90% identical to the amino acid sequence of SEQ ID NO: 43 and an amino acid sequence at least 90% identical to the amino acid sequence of SEQ ID NO: 46, more preferably an amino acid sequence at least 95% identical to the amino acid sequence of SEQ ID NO: 43 and an amino acid sequence at least 95% identical to the amino acid sequence of SEQ ID NO: 46, more preferably an amino acid sequence at least 98% identical to the amino acid sequence of SEQ ID NO: 43 and an amino acid sequence at least 98% identical to the amino acid sequence of SEQ ID NO: 46, more preferably an amino acid sequence at least 99% identical to the amino acid sequence of SEQ ID NO: 43 and an amino acid sequence at least 99% identical to the amino acid sequence of SEQ ID NO: 46, and most preferably the amino acid sequence of SEQ ID NO: 43 and the amino acid sequence of SEQ ID NO: 46;
   yy. an amino acid sequence at least 85% identical to the amino acid sequence of SEQ ID NO: 47 and an amino acid sequence at least 85% identical to the amino acid sequence of SEQ ID NO: 48, preferably an amino acid sequence at least 90% identical to the amino acid sequence of SEQ ID NO: 47 and an amino acid sequence at least 90% identical to the amino acid sequence of SEQ ID NO: 48, more preferably an amino acid sequence at least 95% identical to the amino acid sequence of SEQ ID NO: 47 and an amino acid sequence at least 95% identical to the amino acid sequence of SEQ ID NO: 48, more preferably an amino acid sequence at least 98% identical to the amino acid sequence of SEQ ID NO: 47 and an amino acid sequence at least 98% identical to the amino acid sequence of SEQ ID NO: 48, more preferably an amino acid sequence at least 99% identical to the amino acid sequence of SEQ ID NO: 47 and an amino acid sequence at least 99% identical to the amino acid sequence of SEQ ID NO: 48, and most preferably the amino acid sequence of SEQ ID NO: 47 and the amino acid sequence of SEQ ID NO: 48;
   zz. an amino acid sequence at least 85% identical to the amino acid sequence of SEQ ID NO: 47 and an amino acid sequence at least 85% identical to the amino acid sequence of SEQ ID NO: 49, preferably an amino acid sequence at least 90% identical to the amino acid sequence of SEQ ID NO: 47 and an amino acid sequence at least 90% identical to the amino acid sequence of SEQ ID NO: 49, more preferably an amino acid sequence at least 95% identical to the amino acid sequence of SEQ ID NO: 47 and an amino acid sequence at least 95% identical to the amino acid sequence of SEQ ID NO: 49, more preferably an amino acid sequence at least 98% identical to the amino acid sequence of SEQ ID NO: 47 and an amino acid sequence at least 98% identical to the amino acid sequence of SEQ ID NO: 49, more preferably an amino acid sequence at least 99% identical to the amino acid sequence of SEQ ID NO: 47 and an amino acid sequence at least 99% identical to the amino acid sequence of SEQ ID NO: 49, and most preferably the amino acid sequence of SEQ ID NO: 47 and the amino acid sequence of SEQ ID NO: 49;
   aaa. an amino acid sequence at least 85% identical to the amino acid sequence of SEQ ID NO: 47 and an amino acid sequence at least 85% identical to the amino acid sequence of SEQ ID NO: 50, preferably an amino acid sequence at least 90% identical to the amino acid sequence of SEQ ID NO: 47 and an amino acid sequence at least 90% identical to the amino acid sequence of SEQ ID NO: 50, more preferably an amino acid sequence at least 95% identical to the amino acid sequence of SEQ ID NO: 47 and an amino acid sequence at least 95% identical to the amino acid sequence of SEQ ID NO: 50, more preferably an amino acid sequence at least 98% identical to the amino acid sequence of SEQ ID NO: 47 and an amino acid sequence at least 98% identical to the amino acid sequence of SEQ ID NO: 50, more preferably an amino acid sequence at least 99% identical to the amino acid sequence of SEQ ID NO: 47 and an amino acid sequence at least 99% identical to the amino acid sequence of SEQ ID NO: 50, and most preferably the amino acid sequence of SEQ ID NO: 47 and the amino acid sequence of SEQ ID NO: 50;
   bbb. an amino acid sequence at least 85% identical to the amino acid sequence of SEQ ID NO: 51 and an amino acid sequence at least 85% identical to the amino acid sequence of SEQ ID NO: 52, preferably an amino acid sequence at least 90% identical to the amino acid sequence of SEQ ID NO: 51 and an amino acid sequence at least 90% identical to the amino acid sequence of SEQ ID NO: 52, more preferably an amino acid sequence at least 95% identical to the amino acid sequence of SEQ ID NO: 51 and an amino acid sequence at least 95% identical to the amino acid sequence of SEQ ID NO: 52, more preferably an amino acid sequence at least 98% identical to the amino acid sequence of SEQ ID NO: 51 and an amino acid sequence at least 98% identical to the amino acid sequence of SEQ ID NO: 52, more preferably an amino acid sequence at least 99% identical to the amino acid sequence of SEQ ID NO: 51 and an amino acid sequence at least 99% identical to the amino acid sequence of SEQ ID NO: 52, and most preferably the amino acid sequence of SEQ ID NO: 51 and the amino acid sequence of SEQ ID NO: 52;
   ccc. an amino acid sequence at least 85% identical to the amino acid sequence of SEQ ID NO: 51 and an amino acid sequence at least 85% identical to the amino acid sequence of SEQ ID NO: 53, preferably an amino acid sequence at least 90% identical to the amino acid sequence of SEQ ID NO: 51 and an amino acid sequence at least 90% identical to the amino acid sequence of SEQ ID NO: 53, more preferably an amino acid sequence at least 95% identical to the amino acid sequence of SEQ ID NO: 51 and an amino acid sequence at least 95% identical to the amino acid sequence of SEQ ID NO: 53, more preferably an amino acid sequence at least 98% identical to the amino acid sequence of SEQ ID NO: 51 and an amino acid sequence at least 98% identical to the amino acid sequence of SEQ ID NO: 53, more preferably an amino acid sequence at least 99% identical to the amino acid sequence of SEQ ID NO: 51 and an amino acid sequence at least 99% identical to the amino acid sequence of SEQ ID NO: 53, and most preferably the amino acid sequence of SEQ ID NO: 51 and the amino acid sequence of SEQ ID NO: 53;
   ddd. an amino acid sequence at least 85% identical to the amino acid sequence of SEQ ID NO: 51 and an amino acid sequence at least 85% identical to the amino acid sequence of SEQ ID NO: 54, preferably an amino acid sequence at least 90% identical to the amino acid sequence of SEQ ID NO: 51 and an amino acid sequence at least 90% identical to the amino acid sequence of SEQ ID NO: 54, more preferably an amino acid sequence at least 95% identical to the amino acid sequence of SEQ ID NO: 51 and an amino acid sequence at least 95% identical to the amino acid sequence of SEQ ID NO: 54, more preferably an amino acid sequence at least 98% identical to the amino acid sequence of SEQ ID NO: 51 and an amino acid sequence at least 98% identical to the amino acid sequence of SEQ ID NO: 54, more preferably an amino acid sequence at least 99% identical to the amino acid sequence of SEQ ID NO: 51 and an amino acid sequence at least 99% identical to the amino acid sequence of SEQ ID NO: 54, and most preferably the amino acid sequence of SEQ ID NO: 51 and the amino acid sequence of SEQ ID NO: 54;
   eee. an amino acid sequence at least 85% identical to the amino acid sequence of SEQ ID NO: 55 and an amino acid sequence at least 85% identical to the amino acid sequence of SEQ ID NO: 56, preferably an amino acid sequence at least 90% identical to the amino acid sequence of SEQ ID NO: 55 and an amino acid sequence at least 90% identical to the amino acid sequence of SEQ ID NO: 56, more preferably an amino acid sequence at least 95% identical to the amino acid sequence of SEQ ID NO: 55 and an amino acid sequence at least 95% identical to the amino acid sequence of SEQ ID NO: 56, more preferably an amino acid sequence at least 98% identical to the amino acid sequence of SEQ ID NO: 55 and an amino acid sequence at least 98% identical to the amino acid sequence of SEQ ID NO: 56, more preferably an amino acid sequence at least 99% identical to the amino acid sequence of SEQ ID NO: 55 and an amino acid sequence at least 99% identical to the amino acid sequence of SEQ ID NO: 56, and most preferably the amino acid sequence of SEQ ID NO: 55 and the amino acid sequence of SEQ ID NO: 56;
   fff. an amino acid sequence at least 85% identical to the amino acid sequence of SEQ ID NO: 57, preferably an amino acid sequence at least 90% identical to the amino acid sequence of SEQ ID NO: 57, more preferably an amino acid sequence at least 95% identical to the amino acid sequence of SEQ ID NO: 57, more preferably an amino acid sequence at least 98% identical to the amino acid sequence of SEQ ID NO: 57, more preferably an amino acid sequence at least 99% identical to the amino acid sequence of SEQ ID NO: 57, and most preferably the amino acid sequence of SEQ ID NO: 57; or
   ggg. an amino acid sequence at least 85% identical to the amino acid sequence of SEQ ID NO: 58, preferably an amino acid sequence at least 90% identical to the amino acid sequence of SEQ ID NO: 58, more preferably an amino acid sequence at least 95% identical to the amino acid sequence of SEQ ID NO: 58, more preferably an amino acid sequence at least 98% identical to the amino acid sequence of SEQ ID NO: 58, more preferably an amino acid sequence at least 99% identical to the amino acid sequence of SEQ ID NO: 58, and most preferably the amino acid sequence of SEQ ID NO: 58.
24. The construct according to any one of the preceding items, further comprising an N-terminal amino acid sequence comprising one or two of the following ((a) and (b)): (a) a leader sequence comprising the amino acid sequence of SEQ ID NO: 30, optionally followed by a first linker sequence, and (b) a flag tag comprising the amino acid sequence of SEQ ID NO: 61, optionally followed by a second linker sequence.
25. A pharmaceutical composition comprising the construct according to any one of the preceding items.
26. A pharmaceutical composition according to item 25 or a construct according to any one of items 1-12, for use in the treatment of cancer.
27. The pharmaceutical composition or construct for use according to item 26, wherein the cancer is a cancer expressing CD40.
28. A nucleic acid, or a set of nucleic acids, encoding the construct of any one of items 1-24.
29. A recombinant cell containing a nucleic acid, or a set of nucleic acids, according to item 28 and expressing the construct of any one of items 1-24.
30. A method for producing a construct of any one of items 1-24, the method comprising expressing said construct in a recombinant cell according to item 29 from the nucleic acid or set of nucleic acids according to item 28, wherein the method optionally further comprises purifying and formulating said construct into a pharmaceutical composition according to item 25.

### BRIEF DESCRIPTION OF THE DRAWINGS

FIG. 1:
(A-E) Characterization of anti-CD40 antibodies. (A) Effect of anti-CD40 IgG antibodies on CD40L binding. CD40-expressing cells were incubated with 200 ng/ml GpL-CD40L, a fusion protein of soluble CD40L and Gaussia princeps luciferase (GpL), and 5 μg/ml anti-CD40 antibody for 1 hour. After removing the unbound molecules by washing 3 times with PBS, the cell-bound GpL-CD40L amount was quantified using a luciferase assay and normalized with respect to the control without antibodies. Shown is the mean of three independent experiments. The antibody treated groups were compared to the control group by ANOVA. ***p<0.001; *p<0.05; ns not significant. (B) U20S cells were treated with 10 μg/ml of the indicated antibodies along with HEK293 transfectants expressing memCD40L (293-mCD40L) or empty vector (293-EV). Next day, CD40-induced IL8 production was evaluated by ELISA. (C) Specific binding of anti-CD40-IgG1-GpL fusion proteins to HT1080 cells stably expressing (HT1080-CD40). Binding of anti-CD40-IgG1-GpL fusion proteins to conventional HT1080 cells has been considered as unspecific binding, Binding of anti-CD40-IgG1-GpL fusion proteins to HT1080-CD40 cells resulted in total binding and specific CD40 binding (open circles) was calculated by subtraction of the unspecific binding values (filled circles) of the corresponding total binding values (filled squares). Corresponding analysis of binding of GpL-TNC-CD40L to CD40 (lower panel, right diagram) served as positive control. (D) Domain architecture of CD40 and CD40 deletion mutants used in following part “E”. (E) Protein G-coated ELISA plates were loaded with anti-CD40 antibodies. Protein G/anti-CD40 antibody complexes were finally incubated with C-terminal deletion mutants of the CD40 ectodomain harboring a C-terminal Gaussia princeps luciferase (GpL) reporter domain (see “D”) or TNFR2(ed)-GpL as negative control. Specific binding of the CD40 deletion mutant molecules were finally obtained by the subtraction of the unspecific TNFR2(ed)-GpL binding values from the total binding values of the various CD40-GpL fusion proteins.

FIG. 2: FcγR binding boosts anti-CD40 antibody-triggered IL8 production. (A) Specific binding of IgG1, IgG2, IgG4 and IgG1(N297A) variants of G28.5 to murine FcγRIIb. (B) CD40-responsive U20S were challenged with IgG1, IgG2, IgG4 and IgG1(N297A) variants of the indicated anti-CD40 antibodies along with HEK293 cells transfected with empty vector (EV) or expression plasmids encoding murine FcγRIIb. Next day, cell supernatants were analyzed for IL8 production as readout of CD40 activation. (C) Co-cultures of U20S and murine A20 cells, which endogenously express FcγRs, were stimulated with the anti-CD40 antibody CD40(Apexi)-IgG1 and the next day, IL8 production was again determined by ELISA.

FIG. 3: FcγR binding enhances anti-CD40 antibody-induced p100 processing. (A) U20S cells were stimulated overnight with HEK293 cells transfected with empty vector (EV) or an expression plasmid encoding murine FcγRIIb and 150 ng/ml of the indicated antibodies. Total cell lysates were analyzed for p100 processing. (B) U2OS cells were stimulated overnight with murine L929 cells not expressing FcγRs or murine A20 cells expressing endogenously FcγRIIb and again 150 ng/ml of the indicated antibodies. Total cell lysates were analyzed for p100 processing.

FIG. 4: Oligovalent anti-CD40-IgG1(N297A) variants and anti-CD40-IgG1(N297A)-HC:scFvCD40 fusion proteins exert FcγR-independent CD40 agonism. (A) Domain architecture (upper panel) and Western blotting (lower panel) of the genetically engineered oligomerized anti-CD40-IgG1(N297A)-TNC and anti-CD40-IgG1(N297A-RGY) antibody variants and the tetravalent anti-CD40-IgG1(N297A)-HC:scFvCD40 fusion proteins. (B) HT1080-CD40 cells, which strongly produce the NFκB-regulated cytokine IL8 after CD40 stimulation, were stimulated overnight with the different anti-CD40 antibody variants and finally the IL8 production was recorded by ELISA. HT1080 cells were also challenged with membrane CD40L transfected HEK293 cells. The resulting IL8 production was defined as maximal und used to define the possible half-maximal IL8 response. Shown are the mean values derived of four independent experiments.

FIG. 5: Agonism of oligovalent anti-CD40-IgG1(N297A) variants and anti-CD40-IgG1(N297A)-HC:scFvCD40 fusion proteins is aggregation-independent. (A) The indicated constructs were purified by affinity chromatography on anti-Flag agarose and purity was evaluated by SDAS-PAGE and silver staining. (B) Gel filtration analysis of the purified antibody fusion proteins. Dotted arrows indicate Flag peptide remained from the affinity purification. (C) IL8 induction in U2OS by purified IgG1(N297A)-scFv fusion proteins.

FIG. 6: Oligomeric sdAb:CD40 variants display strong CD40 agonists. (A) Domain architecture of oligomeric sdAb:CD40 variants. (B) Western blot analysis sdAb:CD40 variants. (C) U2OS cells were stimulated overnight with the different VHH:CD40 variants and finally IL8 production were determined by ELISA. (D, E) The indicated constructs were purified by affinity chromatography and analyzed by SDS-PAGE (D) and gel filtration (E).

FIG. 7: CD40-specific antibody construct antiCD40(Seli)-N297A-HC:scFvSeli with intrinsic agonism trigger maturation of iDCs. Immature monocyte-derived dendritic cells (iDCs) were generated by cultivation of monocytes for 7 days with GM-CSF/IL4. iDCs were then treated with 200 ng/ml of antiCD40(Seli)-N297A-HC:scFvSeli and were analyzed after two days by flow cytometry for the cell surface expression of CD14 and CD83.

FIG. 8: Induction of iDC maturation by VHH(V12t)-based multivalent CD40-specific constructs. Immature monocyte-derived dendritic cells (iDCs) were again generated by cultivation of monocytes for 7 days with GM-CSF/IL4. (A) iDCs were treated with 200 ng/ml of the indicated constructs and were then analyzed after two days by flow cytometry for the cell surface expression of CD14 and CD83. (B) iDCs were treated with 200 ng/ml of the indicated constructs and were analyzed next day by western blotting for the presence of the indicated proteins.

FIG. 9: Multivalent variants of the CD40-specific antibody antiCD40(Seli)-N297A with intrinsic agonism trigger IL8 production in iDCs. Immature monocyte-derived dendritic cells (iDCs) were generated by cultivation of monocytes for 7 days with GM-CSF/IL4. iDCs were then treated with 500 ng/ml of the indicated variants of antiCD40(Seli). Next day, cell culture supernatants were analyzed by IL8 ELISA.

FIG. 10: Stimulation of IL8 production by VHH(V12t)-based multivalent CD40-specific constructs. Immature monocyte-derived dendritic cells (iDCs) were again generated by cultivation of monocytes for 7 days with GM-CSF/IL4. iDCs were treated with 8, 40 or 200 ng/ml of the indicated constructs overnight and finally cell culture supernatants were analyzed for their IL8 content by ELISA. * p<0.05; ** p<0.01, *** p<***.

FIG. 11: Intrinsic agonism of IgG1(N297A)-scaffold based tetra-, hexa- and octavalent sdAb:CD40(V12t) variants. Upper panel: Domain architecture of constructs. Lower panel: U2OS cells were stimulated overnight with the different VHH:CD40-IgG1(N297A) variants and finally IL8 production were determined by ELISA. Stimulation with soluble Flag-CD40L oligomerized with the anti-Flag mAb M2 served as a positive control.

FIG. 12: Intrinsic agonism of IgG1(N297A)-scaffold based hexa-, octa- and decavalent sdAb:CD40(V12t) variants. Upper panel: Domain architecture of constructs. Lower panel: U2OS cells were stimulated overnight with the different VHH:CD40-IgG1(N297A) variants and finally IL8 production were determined by ELISA. Stimulation with soluble Flag-CD40L oligomerized with the anti-Flag mAb M2 served as a positive control.

FIG. 13: Intrinsic agonism of IgG1(N297A)-scaffold based octa- and deca sdAb:CD40(V12t) variants. Upper panel: Domain architecture of constructs. Lower panel: U2OS cells were stimulated overnight with the different VHH:CD40-IgG1(N297A) variants and finally IL8 production were determined by ELISA. Stimulation with soluble Flag-CD40L oligomerized with the anti-Flag mAb M2 served as a positive control.

FIG. 14: Intrinsic agonism of Fab-scaffold based bi-, tri- and tetravalent sdAb:CD40(V12t) variants. Upper panel: Domain architecture of constructs. Lower panel: U2OS cells were stimulated overnight with the different VHH:CD40-Fab variants and finally IL8 production was determined by ELISA. Stimulation with soluble Flag-CD40L oligomerized with the anti-Flag mAb M2 served as a positive control.

FIG. 15: Intrinsic agonism of Fc(DANA)-scaffold based bivalent sdAb:CD40(V12t) variants with different VHH domain positioning. Upper panel: Domain architecture of constructs. Lower panel: HT1080-CD40 cells were stimulated overnight with the different VHH:CD40-Fc(DANA) variants containing in total six VHH domains each but only two of them recognizing CD40 and finally IL8 production was determined in cell culture supernatants by ELISA. Stimulation with soluble Flag-CD40L oligomerized with the anti-Flag mAb M2 served as a positive control.

FIG. 16: Intrinsic agonism of Fc(DANA)-scaffold based tetravalent sdAb:CD40(V12t) variants with different VHH domain positioning. Upper panel: Domain architecture of constructs. Lower panel: HT1080-CD40 cells were stimulated overnight with the different VHH:CD40-Fc(DANA) variants and finally IL8 production was determined in cell culture supernatants by ELISA. Stimulation with soluble Flag-CD40L oligomerized with the anti-Flag mAb M2 served as a positive control.

FIG. 17: Intrinsic agonism of Tenascin trimerization (TNC) domain-scaffold based tri-, hexa- and nonavalent sdAb:CD40(V12t) variants. Upper panel: Domain architecture of constructs. Lower panel: U2OS cells were stimulated overnight with the different VHH:CD40-TNC variants and finally IL8 production was determined by ELISA. Stimulation with soluble Flag-CD40L oligomerized with the anti-Flag mAb M2 served as a positive control.

FIG. 18: Intrinsic agonism of Fc(DANA)-scaffold based bi-, tetra- and hexavalent sdAb:CD40(V12t) variants. Upper panel: Domain architecture of constructs. Lower panel: U2OS cells were stimulated overnight with the different VHH:CD40-Fc(DANA) variants and finally IL8 production was determined in cell culture supernatants by ELISA. Stimulation with soluble Flag-CD40L oligomerized with the anti-Flag mAb M2 served as a positive control.

FIG. 19: Intrinsic agonism of Fab-scaffold based tri-, tetra- and pentavalent sdAb:CD40(V12t) variants with one sdAb domain on the opposite part of the scaffold than the other domains. Upper panel: Domain architecture of constructs. Lower panel: U2OS cells were stimulated overnight with the different VHH:CD40-Fab variants and finally IL8 production was determined by ELISA. Stimulation with soluble Flag-CD40L oligomerized with the anti-Flag mAb M2 served as a positive control.

### DETAILED DESCRIPTION OF THE INVENTION

### Definitions and General Techniques

Unless otherwise defined below, the terms used in the present invention shall be understood in accordance with their common meaning known to the person skilled in the art. All publications, patents and patent applications cited herein are hereby incorporated by reference in their entirety for all purposes. Publications referred to herein may be cited by specifying the full literature reference in the text, or by naming the author and the publication year (e.g., “Kuramitsu et al. 2014”) and by specifying the corresponding full literature reference in the “references” section.

All proteins in accordance with the invention, including the antibody constructs of the invention, can be obtained by methods known in the art. Such methods include methods for the production of recombinant proteins. The antibody constructs of the invention can be expressed in recombinant host cells according to the invention. Recombinant host cells of the invention are preferably mammalian cells such as CHO and HEK cells. It will be understood that the antibody constructs of the invention are meant to optionally include a secretion signal peptide sequence. Similarly, the antibody constructs of the invention are meant to also optionally include affinity tags, e.g. in order to facilitate purification, and optional protease cleavage sites between the tag and the antibody construct, e.g. in order to facilitate removal of the tags by protease cleavage. Likewise, it will be understood that the antibody constructs of the invention are meant to optionally include the respective pro-peptides.

It is also understood that any reference to amino acid sequences referred to herein is meant to encompass not only the unmodified amino acid sequence but also typical posttranslational modifications of these amino acid sequences (e.g., glycosylation or deamidation of amino acids, the clipping of particular amino acids or other posttranslational modifications) occurring in cellular expression systems known in the art, including mammalian cells such as CHO and HEK cells.

The term “antibody” as used herein refers to any functional antibody that is capable of specific binding to the antigen of interest, as generally outlined in chapter 7 of Paul, W. E. (Ed.).: Fundamental Immunology 2nd Ed. Raven Press, Ltd., New York 1989, which is incorporated herein by reference. Without particular limitation, the term “antibody” encompasses antibodies from any appropriate source species, including chicken and mammalian such as mouse, goat, non-human primate and human. Preferably, the antibody is a humanized or human antibody. The antibody is preferably a monoclonal antibody which can be prepared by methods well-known in the art. The term “antibody” encompasses an IgG-1, -2, -3, or -4, IgE, IgA, IgM, or IgD isotype antibody. The term “antibody” encompasses monomeric antibodies (such as IgD, IgE, IgG) or oligomeric antibodies (such as IgA or IgM). The term “antibody” also encompasses—without particular limitations—isolated antibodies and modified antibodies such as genetically engineered antibodies, e.g. chimeric, humanized or human antibodies. For example, the terms “IgG1”, “IgG2”, “IgG3” or “IgG4” as used in accordance with the invention encompass chimeric, humanized and human IgG1, IgG2, IgG3 and IgG4, respectively.

As defined herein, an antibody construct of the invention is not a natural IgA or IgM molecule. As used herein, the term “natural IgA or IgM molecule” includes any naturally occurring IgA or IgM molecule as well as chimeric, humanized or human forms thereof. That is, an antibody construct of the invention is neither a naturally occurring IgA or IgM molecule nor a chimeric, humanized or human form thereof. In contrast, it is understood that the term “a natural IgA or IgM molecule” does not include IgA or IgM molecules which have been fused to single-domain antibodies (sdAb) containing binding sites for CD40 or to scFvs containing binding sites for CD40, and it does also not include IgA or IgM molecules, parts of which have been replaced by single-domain antibodies (sdAb) containing binding sites for CD40 or by scFvs containing binding sites for CD40. Thus, in an embodiment of the invention, an antibody construct of the invention can be an IgA or IgM molecule which has been fused to single-domain antibodies (sdAb) containing binding sites for CD40 or to scFvs containing binding sites for CD40, or an IgA or IgM molecule, parts of which have been replaced by single-domain antibodies (sdAb) containing binding sites for CD40 or by scFvs containing binding sites for CD40.

The term single-domain antibody (sdAb) is to be understood in accordance with its known meaning in the art. Single-domain antibodies are oftentimes also referred to as “nanobodies”. Single-domain antibodies can, for instance, be engineered from heavy-chain antibodies such as those found in camelids. Single-domain antibodies engineered from heavy-chain antibodies are oftentimes referred to as “VHH”. Thus, in one preferred embodiment, one, more, or all occurrences of a “single-domain antibody (sdAb)” in connection with the invention can optionally be replaced by “VHH”.

As used herein, an “IgG” can be a naturally occurring IgG or a mutated IgG, as is known in the art.

For IgG1 such as chimeric, humanized or human IgG1, known mutations include, but are not limited to: N297A, N297D, N297Q, N297G, E233P, L234A, L234F, L235A, L235E, P331S, P329A, P329G, P331S, P238S, LALA (LALA=mutation of Leucine 234 to Alanine and Leucine 235 to Alanine), A330S, G237A, M252Y, S254T, S228P, T256E, M252F, M252S, M252W, M252T, T256S, T256R, T256Q, T256D, H268A, and combinations thereof. Known IgG1 mutations also include E345K or E430G which promote IgG1 hexamerization.

For IgG2 such as chimeric, humanized or human IgG2, known mutations include, but are not limited to: V234A, G237A, P238S, H268A, H268Q, A330S, P331S, P233S, V309L, and combinations thereof.

For IgG3 such as chimeric, humanized or human IgG3, known mutations include, but are not limited to: replacement of the CH2 domain of IgG3 with the CH2 domain of IgG2, removal of Fc glycosylation, mutations of L322, L276, N297, and combinations thereof.

For IgG4 such as chimeric, humanized or human IgG4, known mutations include, but are not limited to: V234A, L235E, LALA (Mutation of Leucine 234 to Alanine and Leucine 235 to Alanine), F234A, P329G, S228P, G237A, P238S, G236 deletion, and combinations thereof.

The nomenclature of the antibodies, antibody fragments and mutations thereof follows the terms as known in the art. It will be understood by a person skilled in the art that in accordance with the invention, this known nomenclature is to be applied also to the antibody constructs of the invention. For example, the known nomenclature is to be applied also to an antibody which forms part of an antibody construct of the invention. For instance, where terms relating antibody mutations such as “N297A” are used herein in connection with an IgG1 which forms part of an antibody construct of the invention, it will be understood that these terms are based on known antibody nomenclature.

Each monomer of an antibody comprises two heavy chains and two light chains, as generally known in the art. Of these, each heavy and light chain comprises a variable domain (termed VH for the heavy chain and VL for the light chain) which is important for antigen binding. These heavy and light chain variable domains comprise (in an N-terminal to C-terminal order) the regions FR1, CDR1, FR2, CDR2, FR3, CDR3, and FR4 (FR, framework region; CDR, complementarity determining region which is also known as hypervariable region). The identification and assignment of the above-mentioned antibody regions within the antibody sequence is generally in accordance with Kabat et al. (Sequences of proteins of immunological interest, U.S. Dept. of Health and Human Services, Public Health Service, National Institutes of Health, Bethesda, Md. 1983), or Chothia et al. (Conformations of immunoglobulin hypervariable regions. Nature. 1989 Dec. 21-28; 342(6252):877-83.), or may be performed by using the IMGTN-QUEST software described in Giudicelli et al. (IMGTN-QUEST, an integrated software program for immunoglobulin and T cell receptor V-J and V-D-J rearrangement analysis. Nucleic Acids Res. 2004 Jul. 1; 32(Web Server issue):W435-40.), which is incorporated herein by reference. Preferably, the antibody regions indicated above are identified and assigned by using the IMGTN-QUEST software.

A “monoclonal antibody” is an antibody from an essentially homogenous population of antibodies, wherein the antibodies are substantially identical in sequence (i.e. identical except for minor fraction of antibodies containing naturally occurring sequence modifications such as amino acid modifications at their N- and C-termini). Unlike polyclonal antibodies which contain a mixture of different antibodies directed to either a single epitope or to numerous different epitopes, monoclonal antibodies are directed to the same epitope and are therefore highly specific. The term “monoclonal antibody” includes (but is not limited to) antibodies which are obtained from a monoclonal cell population derived from a single cell clone, as for instance the antibodies generated by the hybridoma method described in Kohler and Milstein (Nature, 1975 Aug. 7; 256(5517):495-7) or Harlow and Lane (“Antibodies: A Laboratory Manual” Cold Spring Harbor Laboratory Press, Cold Spring Harbor, New York 1988). A monoclonal antibody may also be obtained from other suitable methods, including phage display techniques such as those described in Clackson et al. (Nature. 1991 Aug. 15; 352(6336):624-8) or Marks et al. (J Mol Biol. 1991 Dec. 5; 222(3):581-97). A monoclonal antibody may be an antibody that has been optimized for antigen-binding properties such as decreased Kd values, optimized association and dissociation kinetics by methods known in the art. For instance, Kd values may be optimized by display methods including phage display, resulting in affinity-matured monoclonal antibodies. The term “monoclonal antibody” is not limited to antibody sequences from particular species of origin or from one single species of origin. Thus, the meaning of the term “monoclonal antibody” encompasses chimeric monoclonal antibodies such as humanized monoclonal antibodies and human antibodies.

“Humanized antibodies” are antibodies which contain human sequences and a minor portion of non-human sequences which confer binding specificity to an antigen of interest. Typically, humanized antibodies are generated by replacing hypervariable region sequences from a human acceptor antibody by hypervariable region sequences from a non-human donor antibody (e.g. a mouse, rabbit, rat donor antibody) that binds to an antigen of interest. In some cases, framework region sequences of the acceptor antibody may also be replaced by the corresponding sequences of the donor antibody. In addition to the sequences derived from the donor and acceptor antibodies, a “humanized antibody” may either contain other (additional or substitute) residues or sequences or not. Such other residues or sequences may serve to further improve antibody properties such as binding properties (e.g. to decrease Kd values) and/or immunogenic properties (e.g. to decrease antigenicity in humans). Non-limiting examples for methods to generate humanized antibodies are known in the art, e.g. from Riechmann et al. (Nature. 1988 Mar. 24; 332(6162):323-7) or Jones et al. (Nature. 1986 May 29-Jun. 4; 321(6069):522-5).

The term “human antibody” relates to an antibody containing human variable and constant domain sequences. This definition encompasses antibodies having human sequences bearing single amino acid substitutions or modifications which may serve to further improve antibody properties such as binding properties (e.g. to decrease Kd values) and/or immunogenic properties (e.g. to decrease antigenicity in humans). The term “human antibody” excludes humanized antibodies where a portion of non-human sequences confers binding specificity to an antigen of interest.

An “antigen-binding portion” of an antibody as used herein refers to a portion of an antibody that retains the capability of the antibody to specifically bind to the antigen. This capability can, for instance, be determined by determining the capability of the antigen-binding portion to compete with the antibody for specific binding to the antigen by methods known in the art. The antigen-binding portion may contain one or more fragments of the antibody. Without particular limitation, the antigen-binding portion can be produced by any suitable method known in the art, including recombinant DNA methods and preparation by chemical or enzymatic fragmentation of antibodies. Antigen-binding portions may be Fab fragments, F(ab′) fragments, Fab2 fragments, single-chain variable fragments (scFv), single-domain antibodies, diabodies or any other portion(s) of the antibody that retain the capability of the antibody to specifically bind to the antigen. It will be understood that in accordance with the meaning of the term “Fab2” as known in the art, the term “Fab2” is synonymous with the terms “Fab2”, “Fab2”, and “FAB2”. Thus, for the purposes of the present application, the term “Fab2” is used interchangeably with the terms “Fab2”, “Fab2”, and “FAB2”.

An “antibody construct” according to the invention contains several binding sites for CD40, as defined herein, and is a protein. An antibody construct is capable of binding to its specific target antigen, i.e. CD40. It is understood that an antibody construct according to the invention may be a protein consisting of a single polypeptide chain, or it may be a protein wherein two or more polypeptide chains are linked together. They can be linked together covalently, e.g., by covalent linkages. Such covalent linkages may be one or more disulfide linkages. Alternatively, the covalent linkages may be obtained by chemical conjugation (preferably by chemical conjugation using click chemistry), and/or they may be any other covalent linkage which is known in the art as a suitable link for proteins. Alternatively, an antibody construct according to the invention may be a protein wherein two or more polypeptide chains are linked together non-covalently (e.g., by non-covalent interactions). For example, the IgG1 mutations E345K and/or E430G promote IgG1 hexamerization. According to the invention, such IgG1 mutations can be included in the antibody constructs of the invention, in order to obtain hexameric antibody constructs.

Generally, in connection with all antibody constructs of the invention, it will be understood that the parts of the antibody constructs may be fused using linker sequences. In that case, the antibody construct of the invention will comprise such linker sequences. Suitable linker sequences are known in the art and comprise, for example, peptide linkers, without being limited thereto.

An “antibody construct” according to the invention may an antibody construct which is derivatized or linked to a different molecule. For example, molecules that may be linked to the antibody construct are a molecular label (e.g. a fluorescent, luminescent, colored or radioactive molecule) and/or a pharmaceutical agent.

The meaning of the terms “antigen binding site” and “antigen binding sites” as used herein is known in the art. Typically, an antigen binding site comprises six complementarity-determining regions (CDRs). The six complementarity-determining regions (CDRs) are typically located in the VH and VL domains, i.e. a CDR1, a CDR2 and a CDR3 in the VH (i.e., in the variable domain of the heavy chain) and a CDR1, a CDR2 and a CDR3 in the VL (i.e., in the variable domain of the light chain). Various forms of antigen binding sites are known in the art and include, without limitation, antigen binding sites contained in the VH and VH of antibodies, or antigen binding sites contained in their antibody fragments such as scFvs.

The terms “Fc gamma receptor” or “FcγR” as used herein refer to any member of the family of proteins that bind the IgG antibody Fc domain and are encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2). An FcγR can be from any organism, including but not limited to humans, mice, rats, rabbits, and monkeys, and is preferably from humans. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

As used herein, the term “mutation reducing FcγR binding” in connection with the Fc domain of an IgG of the antibody constructs of the invention encompasses any mutation that reduces binding of the antibody construct to at least one of the Fc gamma receptors as compared to a corresponding reference antibody construct where the mutation is absent from the Fc domain. Whether a mutation reduces FcγR binding to an Fc gamma receptor can be determined by methods known in the art such as comparative surface plasmon resonance measurements of the binding of the antibody construct and the reference antibody construct to the respective immobilized recombinant Fc gamma receptor.

Mutations reducing FcγR binding are well known in the art.

For IgG1 such as chimeric, humanized or human IgG1, known mutations reducing FcγR binding include, but are not limited to: N297A, N297D, N297Q, N297G, E233P, L234A, L234F, L235A, L235E, P331S, P329A, P329G, P331S, P238S, LALA (LALA=mutation of Leucine 234 to Alanine and Leucine 235 to Alanine), A330S, G237A, M252Y, S254T, S228P, T256E, M252F, M252S, M252W, M252T, T256S, T256R, T256Q, T256D, H268A, DANA (=D265A and N297A),and combinations thereof.

For IgG2 such as chimeric, humanized or human IgG2, known mutations reducing FcγR binding include, but are not limited to: V234A, G237A, P238S, H268A, H268Q, A330S, P331S, P233S, V309L, and combinations thereof.

For IgG3 such as chimeric, humanized or human IgG3, known mutations reducing FcγR binding include, but are not limited to: replacement of the CH2 domain of IgG3 with the CH2 domain of IgG2, removal of Fc glycosylation, mutations of L322, L276, N297, and combinations thereof.

For IgG4 such as chimeric, humanized or human IgG4, known mutations reducing FcγR binding include, but are not limited to: V234A, L235E, LALA (Mutation of Leucine 234 to Alanine and Leucine 235 to Alanine), F234A, P329G, S228P, G237A, P238S, G236 deletion, and combinations thereof.

The term “CD40 Agonist” refers to a molecule that is able to trigger CD40 signal transduction or certain aspects of CD40 signal transduction (e.g. one pathway of the collection of pathways engaged in memCD40L-induced CD40 signal transduction). CD40 signal transduction can be triggered by CD40-ligand (CD40L/CD154), a trimer transmembrane protein of the TNF superfamily (TNFSF), which occurs in two forms, namely as transmembrane CD40L (memCD40L) or soluble CD40L (sCD40L). memCD40L is predominantly expressed on activated CD4+ T cells, and sCD40L is released from memCD40L by proteolytic processing through, including but not limited to, metalloproteases.

CD40L also binds to integrins αMβ2, α5β1, α4β1, αIIbβ3 und αv33 (Aloui et al., 2014; Takada et al., 2019). The binding of CD40L to the integrins is not competitive with CD40, but rather leads to ternary CD40L-CD40-integrin complexes. Binding of sCD40L to CD40 and an integrin (α5β1) stimulated the classical NFκB signaling and B cell activation (Takada et al., 2019).

Hence, whether an antibody construct is an CD40 agonist can be determined by methods known in the art, including, without limitation, methods for measuring IL8 secretion in cells expressing CD40 (e.g. by ELISA), and methods for measuring p100, the precursor protein of the NFκB-transcription factor subunit p52. Cells expressing CD40 are known in the art and are available and include, for instance, U2OS or HT-1080-CD40 cells but also B-cells, dendritic cells and macrophages.

The cancers to be treated according to the present invention are preferably solid cancers. A “solid cancer” is a cancer which forms one or more solid tumors. Such solid cancers forming solid tumors are generally known in the art. The term “solid cancer” encompasses both a primary tumor formed by the cancer and possible secondary tumors, which are also known as metastases. Known solid cancers to be treated according to the invention include, but are not limited to melanoma, colorectal cancer, prostate cancer, head and neck cancer, urothelial cancer, stomach cancer, pancreatic cancer, liver cancer, testis cancer, ovarian cancer, endometrial cancer, cervical cancer, brain cancer, breast cancer, gastric cancer, renal cell carcinoma, Ewing's sarcoma, non-small cell lung cancer and small cell lung cancer.

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). Preferably, an assessment of whether or not a therapeutic treatment works can be made based on a classification of responders and non-responders by using the response evaluation criteria in solid tumours, version 1.1 (RECIST v1.1) (Eisenhauer et al.: New response evaluation criteria in solid tumours: revised RECIST guideline (version 1.1). In: Eur. J. Cancer. 45, No. 2, January 2009, pp. 228-47). Alternatively, or additionally, an assessment of whether or not a therapeutic treatment works can be made based on known clinical indicators of cancer progression.

The treatment of cancer according to the invention can be a first-line therapy, a second-line therapy or a third-line therapy or a therapy that is beyond third-line therapy. The meaning of these terms is known in the art and in accordance with the terminology that is commonly used by the US National Cancer Institute.

Whether a cancer is a cancer expressing CD40 can be determined by methods known in the art, including, without limitation, immunohistochemistry methods using anti-CD40 antibodies to analyse a biopsy sample of the cancer or flow cytometry or binding studies with CD40-specific reagents (CD40L, antibody).

In accordance with the present invention, each occurrence of the term “comprising” may optionally be substituted with the term “consisting of”.

### Methods and Techniques

Generally, unless otherwise defined herein, the methods used in the present invention (e.g. cloning methods or methods relating to antibodies) are performed in accordance with procedures known in the art, e.g. the procedures described in Sambrook et al. (“Molecular Cloning: A Laboratory Manual.”, 2nd Ed., Cold Spring Harbor Laboratory Press, Cold Spring Harbor, New York 1989), Ausubel et al. (“Current Protocols in Molecular Biology.” Greene Publishing Associates and Wiley Interscience; New York 1992), and Harlow and Lane (“Antibodies: A Laboratory Manual” Cold Spring Harbor Laboratory Press, Cold Spring Harbor, New York 1988), all of which are incorporated herein by reference.

Protein-protein binding, such as binding of antibodies to their respective target proteins, can be assessed by methods known in the art. Protein-protein binding is preferably assessed by surface plasmon resonance spectroscopy measurements, flow cytometry and Gaussia princeps luciferase (GpL) fusion proteins of the proteins of interest (CD40, antibody constructs etc.).

Sequence Alignments of sequences according to the invention are performed by using the BLAST algorithm (see Altschul et al. (1990) “Basic local alignment search tool.” Journal of Molecular Biology 215. p. 403-410.; Altschul et al.: (1997) Gapped BLAST and PSI-BLAST: a new generation of protein database search programs. Nucleic Acids Res. 25:3389-3402.). Appropriate parameters for sequence alignments of short peptides by the BLAST algorithm, which are suitable for peptide antigens in accordance with the invention, are known in the art. Most software tools using the BLAST algorithm automatically adjust the parameters for sequence alignments for a short input sequence. In one embodiment, the following parameters are used: Max target sequences 10; Word size 3; BLOSUM 62 matrix; gap costs: existence 11, extension 1; conditional compositional score matrix adjustment. Thus, when used in connection with sequences, terms such as “identity” or “identical” preferably refer to the identity value obtained by using the BLAST algorithm.

### Preparation of Pharmaceutical Compositions of the Invention

Pharmaceutical compositions of the present invention are prepared in accordance with known standards for the preparation of pharmaceutical compositions. For instance, the pharmaceutical compositions are prepared in a way that they can be stored and administered appropriately. The pharmaceutical compositions of the invention may therefore comprise pharmaceutically acceptable components such as carriers, excipients and/or stabilizers. Such pharmaceutically acceptable components are not toxic in the amounts used when administering the pharmaceutical composition to a human patient. The pharmaceutical acceptable components added to the pharmaceutical compositions may depend on the chemical nature of the active ingredients present in the composition, the particular intended use of the pharmaceutical compositions and the route of administration. In general, the pharmaceutically acceptable components used in connection with the present invention are used in accordance with knowledge available in the art, e.g. from Remington's Pharmaceutical Sciences, Ed. AR Gennaro, 20th edition, 2000, Williams & Wilkins, PA, USA.

Sequences Preferred amino acid sequences referred to in the present application can be independently selected from the following sequences. The sequences are represented in an N-terminal to C-terminal order; and they are represented in the one-letter amino acid code.

The following non-limiting exemplary sequences were used in the experimental examples of the present application:

TABLE 1
Amino acid sequences of heavy and light chain variants of CD40-specific antibody
constructs.

| Plasmid No. | Plasmid name | Encoded amino acidsequence (SEQ ID NO) |
|---|---|---|
| 1 | anti-CD40(G28.5)- | DIVMTQNPLSLPVSLGDEASISCRSSQSLENSNGNTFLNWFFQ |
| | Flag-VL-pCR3 | KPGQSPQLLIYRVSNRFSGVPDRFSGSGSGTDFTLKISRVEAE |
| | | DLGVYFCLQVTHVPYTFGGGTTLEIKGSEIKRTVAAPSVFIFPPS |
| | | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC (SEQ ID NO: 1) |
| 2 | anti-CD40(G28.5)- | DIQLQQSGPGLVKPSQSLSLTCSVTGYSITTNYNWNWIRQFPG |
| | Flag-VH-N297A- | NKLEWMGYIRYDGTSEYTPSLKNRVSITRDTSMNQFFLRLTSVT |
| | TNC-pCR3 | PEDTATYYCARLDYWGQGTLVTVSSGSSSASTKGPSVFPLAP |
| | | SSKSTSGGTAALGCLVKDYFPEPVTVSWNSGALTSGVHTFPA |
| | | VLQSSGLYSLSSVVTVPSSSLGTQTYICNVNHKPSNTKVDKKV |
| | | EPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQ |
| | | PREPQVYTLPPSRDELTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKLEDIACGCAAAPDIKDLLSRLEEL |
| | | EGLVSSLREQGTG (SEQ ID NO: 2) |
| 3 | anti-CD40(G28.5)- | DIQLQQSGPGLVKPSQSLSLTCSVTGYSITTNYNWNWIRQFPG |
| | Flag-VH- | NKLEWMGYIRYDGTSEYTPSLKNRVSITRDTSMNQFFLRLTSVT |
| | IgG1(A + RGY)- | PEDTATYYCARLDYWGQGTLVTVSSGSSSASTKGPSVFPLAP |
| | pCR3 | SSKSTSGGTAALGCLVKDYFPEPVTVSWNSGALTSGVHTFPA |
| | | VLQSSGLYSLSSVVTVPSSSLGTQTYICNVNHKPSNTKVDKKV |
| | | EPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQ |
| | | PRRPQVYTLPPSRDELTKNQVSLTCLVKGFYPSDIAVEWESN |
| | | GQPENNYKTTPPVLDSDGSFFLYSKLTVDKSRWQQGNVFSCS |
| | | VMHGALHNHYTQKYLSLSPGK (SEQ ID NO: 3) |
| 4 | anti-CD40(G28.5)- | DIQLQQSGPGLVKPSQSLSLTCSVTGYSITTNYNWNWIRQFPG |
| | Flag-VH-N297A- | NKLEWMGYIRYDGTSEYTPSLKNRVSITRDTSMNQFFLRLTSVT |
| | scFv-anti- | PEDTATYYCARLDYWGQGTLVTVSSGSSSASTKGPSVFPLAP |
| | CD40(G28.5)-pCR3 | SSKSTSGGTAALGCLVKDYFPEPVTVSWNSGALTSGVHTFPA |
| | | VLQSSGLYSLSSVVTVPSSSLGTQTYICNVNHKPSNTKVDKKV |
| | | EPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQ |
| | | PREPQVYTLPPSRDELTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGKLEDIVMTQNPLSLPVSLGDEASI |
| | | SCRSSQSLENSNGNTFLNWFFQKPGQSPQLLIYRVSNRFSGVP |
| | | DRFSGSGSGTDFTLKISRVEAEDLGVYFCLQVTHVPYTFGGGT |
| | | TLEIKGGGGSGGGGSGGGGSDIQLQQSGPGLVKPSQSLSLTC |
| | | SVTGYSITTNYNWNWIRQFPGNKLEWMGYIRYDGTSEYTPSLK |
| | | NRVSITRDTSMNQFFLRLTSVTPEDTATYYCARLDYWGQGTLV |
| | | TVSS (SEQ ID NO: 4) |
| 5 | anti-CD40(Lob7.4)- | DIQMTQTTSSLSASLGDRVTITCSASQGINNYLNWYQQKPDGT |
| | Flag-VL-pCR3 | VKLLIYYTSSLHSGVPSRFSGSGSGTDYSLTISNLEPEDIATYYC |
| | | QQYSNLPYTFGGGTKLEIKGSEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC (SEQ ID NO: 5) |
| 6 | anti-CD40(Lob7.4)- | EVQLQQSGPDLVKPGASVKISCKTSGYTFTEYIMHWVKQSHGK |
| | Flag-VH-N297A- | SLEWIGGIIPNNGGTSYNQKFKDKATMTVDKSSSTGYMELRSLT |
| | TNC-pCR3 | SEDSAVYYCTRREVYGRNYYALDYWGQGTLVTVSSRSSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKKVEPKSCDKTHTCPPCPAPELLGGPSVFLFPPKP |
| | | KDTLMISRTPEVTCVVVDVSHEDPEVKFNWYVDGVEVHNAKT |
| | | KPREEQYASTYRVVSVLTVLHQDWLNGKEYKCKVSNKALPAP |
| | | IEKTISKAKGQPREPQVYTLPPSRDELTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGKLEDIACGCAAA |
| | | PDIKDLLSRLEELEGLVSSLREQGTG (SEQ ID NO: 6) |
| 7 | anti-CD40(Lob7.4)- | EVQLQQSGPDLVKPGASVKISCKTSGYTFTEYIMHWVKQSHGK |
| | Flag-VH- | SLEWIGGIIPNNGGTSYNQKFKDKATMTVDKSSSTGYMELRSLT |
| | IgG1(A + RGY)- | SEDSAVYYCTRREVYGRNYYALDYWGQGTLVTVSSRSSSAST |
| | pCR3 | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKKVEPKSCDKTHTCPPCPAPELLGGPSVFLFPPKP |
| | | KDTLMISRTPEVTCVVVDVSHEDPEVKFNWYVDGVEVHNAKT |
| | | KPREEQYASTYRVVSVLTVLHQDWLNGKEYKCKVSNKALPAP |
| | | IEKTISKAKGQPRRPQVYTLPPSRDELTKNQVSLTCLVKGFYP |
| | | SDIAVEWESNGQPENNYKTTPPVLDSDGSFFLYSKLTVDKSR |
| | | WQQGNVFSCSVMHGALHNHYTQKYLSLSPGK (SEQ ID NO: |
| | | 7) |
| 8 | anti-CD40(Lob7.4)- | EVQLQQSGPDLVKPGASVKISCKTSGYTFTEYIMHWVKQSHGK |
| | Flag-VH-N297A- | SLEWIGGIIPNNGGTSYNQKFKDKATMTVDKSSSTGYMELRSLT |
| | scFv-anti- | SEDSAVYYCTRREVYGRNYYALDYWGQGTLVTVSSRSSSAST |
| | CD40(Lob7.4)- | KGPSVFPLAPSSKSTSGGTAALGCLVKDYFPEPVTVSWNSGA |
| | pCR3 | LTSGVHTFPAVLQSSGLYSLSSVVTVPSSSLGTQTYICNVNHK |
| | | PSNTKVDKKVEPKSCDKTHTCPPCPAPELLGGPSVFLFPPKP |
| | | KDTLMISRTPEVTCVVVDVSHEDPEVKFNWYVDGVEVHNAKT |
| | | KPREEQYASTYRVVSVLTVLHQDWLNGKEYKCKVSNKALPAP |
| | | IEKTISKAKGQPREPQVYTLPPSRDELTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGKLEEVQLQQSG |
| | | PDLVKPGASVKISCKTSGYTFTEYIMHWVKQSHGKSLEWIGGII |
| | | PNNGGTSYNQKFKDKATMTVDKSSSTGYMELRSLTSEDSAVY |
| | | YCTRREVYGRNYYALDYWGQGTLVTVSSRSSTKGPKLEEGEF |
| | | SEAQLDIQMTQTTSSLSASLGDRVTITCSASQGINNYLNWYQQ |
| | | KPDGTVKLLIYYTSSLHSGVPSRFSGSGSGTDYSLTISNLEPEDI |
| | | ATYYCQQYSNLPYTFGGGTKLEIK (SEQ ID NO: 8) |
| 9 | anti- | QSVLTQPPSASGTPGQRVTISCTGSSSNIGAGYNVYWYQQLP |
| | CD40(Mitazalimab)- | GTAPKLLIYGNINRPSGVPDRFSGSKSGTSASLAISGLRSEDEA |
| | Flag-VL-pCR3 | DYYCAAWDKSISGLVFGGGTKLTVLGGSEIKRTVAAPSVFIFPP |
| | | SDEQLKSGTASVVCLLNNFYPREAKVQWKVDNALQSGNSQE |
| | | SVTEQDSKDSTYSLSSTLTLSKADYEKHKVYACEVTHQGLSS |
| | | PVTKSFNRGEC (SEQ ID NO: 9) |
| 10 | anti- | EVQLLESGGGLVQPGGSLRLSCAASGFTFSTYGMHWVRQAPG |
| | CD40(Mitazalimab)- | KGLEWLSYISGGSSYIFYADSVRGRFTISRDNSENALYLQMNSL |
| | Flag-VH-N297A- | RAEDTAVYYCARILRGGSGMDLWGQGTLVTVSSRSSSASTKG |
| | TNC-pCR3 | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYASTYRVVSVLTVLHQDWLNGKEYKCKVSNKALPAPIE |
| | | KTISKAKGQPREPQVYTLPPSRDELTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPGKLEDIACGCAAAPDI |
| | | KDLLSRLEELEGLVSSLREQGTG (SEQ ID NO: 10) |
| 11 | anti- | EVQLLESGGGLVQPGGSLRLSCAASGFTFSTYGMHWVRQAPG |
| | CD40(Mitazalimab)- | KGLEWLSYISGGSSYIFYADSVRGRFTISRDNSENALYLQMNSL |
| | Flag-VH- | RAEDTAVYYCARILRGGSGMDLWGQGTLVTVSSRSSSASTKG |
| | IgG1(A + RGY-pCR3 | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYASTYRVVSVLTVLHQDWLNGKEYKCKVSNKALPAPIE |
| | | KTISKAKGQPRRPQVYTLPPSRDELTKNQVSLTCLVKGFYPSD |
| | | IAVEWESNGQPENNYKTTPPVLDSDGSFFLYSKLTVDKSRWQ |
| | | QGNVFSCSVMHGALHNHYTQKYLSLSPGK (SEQ ID NO: 11) |
| 12 | anti- | EVQLLESGGGLVQPGGSLRLSCAASGFTFSTYGMHWVRQAPG |
| | CD40(Mitazalimab)- | KGLEWLSYISGGSSYIFYADSVRGRFTISRDNSENALYLQMNSL |
| | Flag-VH-N297A- | RAEDTAVYYCARILRGGSGMDLWGQGTLVTVSSRSSSASTKG |
| | scFv-anti- | PSVFPLAPSSKSTSGGTAALGCLVKDYFPEPVTVSWNSGALT |
| | CD40(Mitazalimab)- | SGVHTFPAVLQSSGLYSLSSVVTVPSSSLGTQTYICNVNHKPS |
| | pCR3 | NTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYASTYRVVSVLTVLHQDWLNGKEYKCKVSNKALPAPIE |
| | | KTISKAKGQPREPQVYTLPPSRDELTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPGKLEEVQLLESGGGL |
| | | VQPGGSLRLSCAASGFTFSTYGMHWVRQAPGKGLEWLSYISG |
| | | GSSYIFYADSVRGRFTISRDNSENALYLQMNSLRAEDTAVYYCA |
| | | RILRGGSGMDLWGQGTLVTVSSRSSTKGPKLEEGEFSEAQLQ |
| | | SVLTQPPSASGTPGQRVTISCTGSSSNIGAGYNVYWYQQLPGT |
| | | APKLLIYGNINRPSGVPDRFSGSKSGTSASLAISGLRSEDEADY |
| | | YCAAWDKSISGLVFGGGTKLTVLG (SEQ ID NO: 12) |
| 13 | anti- | DIVMTQTPSSASEPVGGTVTIKCQASQSISSRLAWYQQKPGQP |
| | CD40(Apexigen)- | PKLLIYRASTLASGVPSRFKGSGSGTEFTLTISDLECADAATYYC |
| | Flag-VL-pCR3 | QCTGYGISWPIGGGTEVVVKGSEIKRTVAAPSVFIFPPSDEQLK |
| | | SGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC (SEQ ID NO: 13) |
| 14 | anti- | QSLEESGGDLVKPGASLTLTCTASGFSFSSTYVCWVRQAPGK |
| | CD40(Apexigen)- | GLEWIACIYTGDGTNYSASWAKGRFTISKPSSTTVTLQMTSLTP |
| | Flag-VH-N297A- | ADTATYFCARPDITYGFAINFWGPGTLVTVSSRSSSASTKGPSV |
| | TNC-pCR3 | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPELLGGPSVFLFPPKPKDTLMI |
| | | SRTPEVTCVVVDVSHEDPEVKFNWYVDGVEVHNAKTKPREEQ |
| | | YASTYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGKLEDIACGCAAAPDIKDL |
| | | LSRLEELEGLVSSLREQGTG (SEQ ID NO: 14) |
| 15 | anti- | QSLEESGGDLVKPGASLTLTCTASGFSFSSTYVCWVRQAPGK |
| | CD40(Apexigen)- | GLEWIACIYTGDGTNYSASWAKGRFTISKPSSTTVTLQMTSLTP |
| | Flag-VH-(A + RGY)- | ADTATYFCARPDITYGFAINFWGPGTLVTVSSRSSSASTKGPSV |
| | pCR3 | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPELLGGPSVFLFPPKPKDTLMI |
| | | SRTPEVTCVVVDVSHEDPEVKFNWYVDGVEVHNAKTKPREEQ |
| | | YASTYRVVSVLTVLHQDWLNGKEYKCKVSNKALPAPIEKTISK |
| | | AKGQPRRPQVYTLPPSRDELTKNQVSLTCLVKGFYPSDIAVE |
| | | WESNGQPENNYKTTPPVLDSDGSFFLYSKLTVDKSRWQQGN |
| | | VFSCSVMHGALHNHYTQKYLSLSPGK (SEQ ID NO: 15) |
| 16 | anti- | QSLEESGGDLVKPGASLTLTCTASGFSFSSTYVCWVRQAPGK |
| | CD40(Apexigen)- | GLEWIACIYTGDGTNYSASWAKGRFTISKPSSTTVTLQMTSLTP |
| | Flag-VH-N297A- | ADTATYFCARPDITYGFAINFWGPGTLVTVSSRSSSASTKGPSV |
| | scFv-anti- | FPLAPSSKSTSGGTAALGCLVKDYFPEPVTVSWNSGALTSGV |
| | CD40(Apexigen)- | HTFPAVLQSSGLYSLSSVVTVPSSSLGTQTYICNVNHKPSNTK |
| | pCR3 | VDKKVEPKSCDKTHTCPPCPAPELLGGPSVFLFPPKPKDTLMI |
| | | SRTPEVTCVVVDVSHEDPEVKFNWYVDGVEVHNAKTKPREEQ |
| | | YASTYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGKLEQSLEESGGDLVKPG |
| | | ASLTLTCTASGFSFSSTYVCWVRQAPGKGLEWIACIYTGDGTN |
| | | YSASWAKGRFTISKPSSTTVTLQMTSLTPADTATYFCARPDITY |
| | | GFAINFWGPGTLVTVSSRSSTKGPKLEEGEFSEAQLDIVMTQT |
| | | PSSASEPVGGTVTIKCQASQSISSRLAWYQQKPGQPPKLLIYRA |
| | | STLASGVPSRFKGSGSGTEFTLTISDLECADAATYYCQCTGYGI |
| | | SWPIGGGTEVVVK (SEQ ID NO: 16) |
| 17 | anti- | DIQMTQSPSSVSASVGDRVTITCRASQGIYSWLAWYQQKPGKA |
| | CD40(Selicrelumab)- | PNLLIYTASTLQSGVPSRFSGSGSGTDFTLTISSLQPEDFATYYC |
| | Flag-VL-pCR3 | QQANIFPLTFGGGTKVEIKGSEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC (SEQ ID NO: 17) |
| 18 | anti- | QVQLVQSGAEVKKPGASVKVSCKASGYTFTGYYMHWVRQAP |
| | CD40(Selicrelumab)- | GQGLEWMGWINPDSGGTNYAQKFQGRVTMTRDTSISTAYMEL |
| | Flag-VH-N297A- | NRLRSDDTAVYYCARDQPLGYCTNGVCSYFDYWGQGTLVTVS |
| | TNC-pCR3 | SRSSSASTKGPSVFPLAPSSKSTSGGTAALGCLVKDYFPEPV |
| | | TVSWNSGALTSGVHTFPAVLQSSGLYSLSSVVTVPSSSLGTQ |
| | | TYICNVNHKPSNTKVDKKVEPKSCDKTHTCPPCPAPELLGGP |
| | | SVFLFPPKPKDTLMISRTPEVTCVVVDVSHEDPEVKFNWYVDG |
| | | VEVHNAKTKPREEQYASTYRVVSVLTVLHQDWLNGKEYKCKV |
| | | SNKALPAPIEKTISKAKGQPREPQVYTLPPSRDELTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGKLE |
| | | DIACGCAAAPDIKDLLSRLEELEGLVSSLREQGTG (SEQ ID NO: |
| | | 18) |
| 19 | anti- | QVQLVQSGAEVKKPGASVKVSCKASGYTFTGYYMHWVRQAP |
| | CD40(Selicrelumab)- | GQGLEWMGWINPDSGGTNYAQKFQGRVTMTRDTSISTAYMEL |
| | Flag-VH-(A + RGY)- | NRLRSDDTAVYYCARDQPLGYCTNGVCSYFDYWGQGTLVTVS |
| | pCR3 | SRSSSASTKGPSVFPLAPSSKSTSGGTAALGCLVKDYFPEPV |
| | | TVSWNSGALTSGVHTFPAVLQSSGLYSLSSVVTVPSSSLGTQ |
| | | TYICNVNHKPSNTKVDKKVEPKSCDKTHTCPPCPAPELLGGP |
| | | SVFLFPPKPKDTLMISRTPEVTCVVVDVSHEDPEVKFNWYVDG |
| | | VEVHNAKTKPREEQYASTYRVVSVLTVLHQDWLNGKEYKCKV |
| | | SNKALPAPIEKTISKAKGQPRRPQVYTLPPSRDELTKNQVSLT |
| | | CLVKGFYPSDIAVEWESNGQPENNYKTTPPVLDSDGSFFLYS |
| | | KLTVDKSRWQQGNVFSCSVMHGALHNHYTQKYLSLSPGK |
| | | (SEQ ID NO: 19) |
| 20 | anti- | QVQLVQSGAEVKKPGASVKVSCKASGYTFTGYYMHWVRQAP |
| | CD40(Selicrelumab)- | GQGLEWMGWINPDSGGTNYAQKFQGRVTMTRDTSISTAYMEL |
| | Flag-VH-scFv-anti- | NRLRSDDTAVYYCARDQPLGYCTNGVCSYFDYWGQGTLVTVS |
| | CD40(Selicrelumab)- | SRSSSASTKGPSVFPLAPSSKSTSGGTAALGCLVKDYFPEPV |
| | pCR3 | TVSWNSGALTSGVHTFPAVLQSSGLYSLSSVVTVPSSSLGTQ |
| | | TYICNVNHKPSNTKVDKKVEPKSCDKTHTCPPCPAPELLGGP |
| | | SVFLFPPKPKDTLMISRTPEVTCVVVDVSHEDPEVKFNWYVDG |
| | | VEVHNAKTKPREEQYASTYRVVSVLTVLHQDWLNGKEYKCKV |
| | | SNKALPAPIEKTISKAKGQPREPQVYTLPPSRDELTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGKLE |
| | | QVQLVQSGAEVKKPGASVKVSCKASGYTFTGYYMHWVRQAP |
| | | GQGLEWMGWINPDSGGTNYAQKFQGRVTMTRDTSISTAYMEL |
| | | NRLRSDDTAVYYCARDQPLGYCTNGVCSYFDYWGQGTLVTVS |
| | | SRSSTKGPKLEEGEFSEAQLDIQMTQSPSSVSASVGDRVTITC |
| | | RASQGIYSWLAWYQQKPGKAPNLLIYTASTLQSGVPSRFSGSG |
| | | SGTDFTLTISSLQPEDFATYYCQQANIFPLTFGGGTKVEIK (SEQ |
| | | ID NO: 20) |
| 21 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Flag-LC-pCR3 | SLKPEDTAVYYCNMHTFWGQGTQVTVSSGSEIKRTVAAPSVFI |
| | | FPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGEC (SEQ ID NO: 21) |
| 22 | anti- | QVQLQESGGGLVQAGGSLRLSCAASGLVFKRYSMNWYRQPP |
| | CD40(V12)VHH(2x)- | GQQRGLVASISDSGVSTNYADSVKGRFTISRDNAKNIGYLQMN |
| | Flag-LC-pCR3 | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQFEIKRTVAAPSVFI |
| | | FPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGEC (SEQ ID NO: 22) |
| 23 | anti-CD40(V12)- | QVQLQESGGGLVQAGGSLRLSCAASGLVFKRYSMNWYRQPP |
| | VHH(3x)-Flag-LC- | GQQRGLVASISDSGVSTNYADSVKGRFTISRDNAKNIGYLQMN |
| | pCR3 | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGE |
| | | C (SEQ ID NO: 23) |
| 24 | anti-CD40(V12)- | QVQLQESGGGLVQAGGSLRLSCAASGLVFKRYSMNWYRQPP |
| | VHH-Flag-N297A- | GQQRGLVASISDSGVSTNYADSVKGRFTISRDNAKNIGYLQMN |
| | pCR3 | SLKPEDTAVYYCNMHTFWGQGTQVTVSSGSSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTCPPCPAPELLGGPSVFLFPPKPKDTLMISR |
| | | TPEVTCVVVDVSHEDPEVKFNWYVDGVEVHNAKTKPREEQYA |
| | | STYRVVSVLTVLHQDWLNGKEYKCKVSNKALPAPIEKTISKAK |
| | | GQPREPQVYTLPPSRDELTKNQVSLTCLVKGFYPSDIAVEWE |
| | | SNGQPENNYKTTPPVLDSDGSFFLYSKLTVDKSRWQQGNVFS |
| | | CSVMHEALHNHYTQKSLSLSPGK (SEQ ID NO: 24) |
| 25 | anti-CD40(V12)- | QVQLQESGGGLVQAGGSLRLSCAASGLVFKRYSMNWYRQPP |
| | VHH(2x)-Flag- | GQQRGLVASISDSGVSTNYADSVKGRFTISRDNAKNIGYLQMN |
| | N297A-pCR3 | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQFSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTCPPCPAPELLGGPSVFLFPPKPKDTLMISR |
| | | TPEVTCVVVDVSHEDPEVKFNWYVDGVEVHNAKTKPREEQYA |
| | | STYRVVSVLTVLHQDWLNGKEYKCKVSNKALPAPIEKTISKAK |
| | | GQPREPQVYTLPPSRDELTKNQVSLTCLVKGFYPSDIAVEWE |
| | | SNGQPENNYKTTPPVLDSDGSFFLYSKLTVDKSRWQQGNVFS |
| | | CSVMHEALHNHYTQKSLSLSPGK (SEQ ID NO: 25) |
| 26 | anti-CD40(V12)- | QVQLQESGGGLVQAGGSLRLSCAASGLVFKRYSMNWYRQPP |
| | VHH(3x)-N297A- | GQQRGLVASISDSGVSTNYADSVKGRFTISRDNAKNIGYLQMN |
| | pCR3 | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSSSASTKGPSVFPLAPSSKSTSGG |
| | | TAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPELLGGPSVFLFPPKPKDTLMISRTPEVTCVVVDV |
| | | SHEDPEVKFNWYVDGVEVHNAKTKPREEQYASTYRVVSVLTV |
| | | LHQDWLNGKEYKCKVSNKALPAPIEKTISKAKGQPREPQVYT |
| | | LPPSRDELTKNQVSLTCLVKGFYPSDIAVEWESNGQPENNYK |
| | | TTPPVLDSDGSFFLYSKLTVDKSRWQQGNVFSCSVMHEALHN |
| | | HYTQKSLSLSPGK (SEQ ID NO: 26) |
| 27 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Flag-TNC-pCR3 | SLKPEDTAVYYCNMHTFWGQGTQVTVSSGSDYKDDDDKDIAC |
| | | GCAAAPDIKDLLSRLEELEGLVSSFREQGTG (SEQ ID NO: 27) |
| 28 | anti- | QVQLQESGGGLVQAGGSLRLSCAASGLVFKRYSMNWYRQPP |
| | CD40(V12)VHH(2x)- | GQQRGLVASISDSGVSTNYADSVKGRFTISRDNAKNIGYLQMN |
| | TNC-pCR3 | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLDYKDDDDKDIAC |
| | | GCAAAPDIKDLLSRLEELEGLVSSLREQGTG (SEQ ID NO: 28) |
| 29 | anti-CD40(V12)(3x)- | QVQLQESGGGLVQAGGSLRLSCAASGLVFKRYSMNWYRQPP |
| | Flag-TNC-pCR3 | GQQRGLVASISDSGVSTNYADSVKGRFTISRDNAKNIGYLQMN |
| | | SLKPEDTAVYYCNMHTFWGQGTQVTVSSRSGGGGSGGGGSG |
| | | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSDYKDDDDKDIACGCAAAPDIKDLL |
| | | SRLEELEGLVSSLREQGTG (SEQ ID NO: 29) |
| 31 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Fc(DANA)-Flag- | SLKPEDTAVYYCNMHTFWGQGTQVTVSSGSKTHTCPPCPAPE |
| | pCR3 | LLGGPSVFLFPPKPKDTLMISRTPEVTCVVVAVSHEDPEVKFN |
| | | WYVDGVEVHNAKTKPREEQYASTYRVVSVLTVLHQDWLNGK |
| | | EYKCKVSNKALPAPIEKTISKAKGQPREPQVYTLPPSRDELTK |
| | | NQVSLTCLVKGFYPSDIAVEWESNGQPENNYKTTPPVLDSDG |
| | | SFFLYSKLTVDKSRWQQGNVFSCSVMHEALHNHYTQKSLSLS |
| | | PGKEFDYKDDDDKLE (SEQ ID NO: 31) |
| 32 | anti- | QVQLQESGGGLVQAGGSLRLSCAASGLVFKRYSMNWYRQPP |
| | CD40(V12)VHH(2x)- | GQQRGLVASISDSGVSTNYADSVKGRFTISRDNAKNIGYLQMN |
| | Fc(DANA)-2xFlag- | SLKPEDTAVYYCNMHTFWGQGTQVTVSSRSGGGGSGGGGSG |
| | pCR3 | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLGTKTHTCPPCP |
| | | APELLGGPSVFLFPPKPKDTLMISRTPEVTCVVVAVSHEDPEV |
| | | KFNWYVDGVEVHNAKTKPREEQYASTYRVVSVLTVLHQDWL |
| | | NGKEYKCKVSNKALPAPIEKTISKAKGQPREPQVYTLPPSRDE |
| | | LTKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGKDYKDDDDKEFDYKDDDDKLE (SEQ ID NO: 32) |
| 33 | anti- | QVQLQESGGGLVQAGGSLRLSCAASGLVFKRYSMNWYRQPP |
| | CD40(V12)VHH(3x)- | GQQRGLVASISDSGVSTNYADSVKGRFTISRDNAKNIGYLQMN |
| | Fc(DANA)-Flag- | SLKPEDTAVYYCNMHTFWGQGTQVTVSSRSGGGGSGGGGSG |
| | pCR3 | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSKTHTCPPCPAPELLGGPSVFLFPP |
| | | KPKDTLMISRTPEVTCVVVAVSHEDPEVKFNWYVDGVEVHNA |
| | | KTKPREEQYASTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFFLYSKLTVDKS |
| | | RWQQGNVFSCSVMHEALHNHYTQKSLSLSPGKEFDYKDDDD |
| | | KLE (SEQ ID NO: 33) |
| 34 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Fc(DANA)-Flag-anti- | SLKPEDTAVYYCNMHTFWGQGTQVTVSSGSKTHTCPPCPAPE |
| | CD40(V12)VHH- | LLGGPSVFLFPPKPKDTLMISRTPEVTCVVVAVSHEDPEVKFN |
| | pCR3 | WYVDGVEVHNAKTKPREEQYASTYRVVSVLTVLHQDWLNGK |
| | | EYKCKVSNKALPAPIEKTISKAKGQPREPQVYTLPPSRDELTK |
| | | NQVSLTCLVKGFYPSDIAVEWESNGQPENNYKTTPPVLDSDG |
| | | SFFLYSKLTVDKSRWQQGNVFSCSVMHEALHNHYTQKSLSLS |
| | | PGKEFDYKDDDDKLEQVQLQESGGGLVQAGGSLRLSCAASGL |
| | | VFKRYSMNWYRQPPGQQRGLVASISDSGVSTNYADSVKGRFT |
| | | ISRDNAKNIGYLQMNSLKPEDTAVYYCNMHTFWGQGTQVTVS |
| | | S (SEQ ID NO: 34) |
| 35 | anti- | QVQLQESGGGLVQAGGSLRLSCAASGLVFKRYSMNWYRQPP |
| | CD40(V12)VHH(3x)- | GQQRGLVASISDSGVSTNYADSVKGRFTISRDNAKNIGYLQMN |
| | Fc(DANA)-Flag- | SLKPEDTAVYYCNMHTFWGQGTQVTVSSRSGGGGGGGGSG |
| | anti- | GGGSGGGGSGGGGSQVQLQESGGGLVQAGGSLRLSCAASG |
| | CD40(V12)VHH- | LVFKRYSMNWYRQPPGQQRGLVASISDSGVSTNYADSVKGRF |
| | pCR3 | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSKTHTCPPCPAPELLGGPSVFLFPP |
| | | KPKDTLMISRTPEVTCVVVAVSHEDPEVKFNWYVDGVEVHNA |
| | | KTKPREEQYASTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFFLYSKLTVDKS |
| | | RWQQGNVFSCSVMHEALHNHYTQKSLSLSPGKEFDYKDDDD |
| | | KLEQVQLQESGGGLVQAGGSLRLSCAASGLVFKRYSMNWYR |
| | | QPPGQQRGLVASISDSGVSTNYADSVKGRFTISRDNAKNIGYL |
| | | QMNSLKPEDTAVYYCNMHTFWGQGTQVTVSS (SEQ ID NO: |
| | | 35) |
| 36 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Flag-FAB1(1-108)- | SLKPEDTAVYYCNMHTFWGQGTQVTVSSGSSSASTKGPSVFP |
| | pCR3 | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHT (SEQ ID NO: 36) |
| 37 | anti- | QVQLQESGGGLVQAGGSLRLSCAASGLVFKRYSMNWYRQPP |
| | CD40(V12)VHH(2x) | GQQRGLVASISDSGVSTNYADSVKGRFTISRDNAKNIGYLQMN |
| | Flag-FAB1(1-108)- | SLKPEDTAVYYCNMHTFWGQGTQVTVSSRSGGGGSGGGGSG |
| | pCR3 | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQFSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHT (SEQ ID NO: 37) |
| 38 | anti- | QVQLQESGGGLVQAGGSLRLSCAASGLVFKRYSMNWYRQPP |
| | CD40(V12)VHH(3x)- | GQQRGLVASISDSGVSTNYADSVKGRFTISRDNAKNIGYLQMN |
| | Flag-FAB1(1-108)- | SLKPEDTAVYYCNMHTFWGQGTQVTVSSRSGGGGSGGGGSG |
| | pCR3 | GGGSGGGGSGGGGSQVQLQESGGGLVQAGGSLRLSCAASG |
| | | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSSSASTKGPSVFPLAPSSKSTSGG |
| | | TAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HT (SEQ ID NO: 38) |
| 39 | anti- | QVQLQESGGGLVQAGGSLRLSCAASGLVFKRYSMNWYRQPP |
| | CD40(V12)VHH- | GQQRGLVASISDSGVSTNYADSVKGRFTISRDNAKNIGYLQMN |
| | Flag-FAB1(1-108)- | SLKPEDTAVYYCNMHTFWGQGTQVTVSSGSSSASTKGPSVFP |
| | anti- | LAPSSKSTSGGTAALGCLVKDYFPEPVTVSWNSGALTSGVHT |
| | CD40(V12)VHH- | FPAVLQSSGLYSLSSVVTVPSSSLGTQTYICNVNHKPSNTKVD |
| | pCR3 | KKVEPKSCDKTHTQVQLQESGGGLVQAGGSLRLSCAASGLVF |
| | | KRYSMNWYRQPPGQQRGLVASISDSGVSTNYADSVKGRFTIS |
| | | RDNAKNIGYLQMNSLKPEDTAVYYCNMHTFWGQGTQVTVSS |
| | | (SEQ ID NO: 39) |
| 40 | anti-CD40(V12) | QVQLQESGGGLVQAGGSLRLSCAASGLVFKRYSMNWYRQPP |
| | VHH(2x)-Flag-FAB1 | GQQRGLVASISDSGVSTNYADSVKGRFTISRDNAKNIGYLQMN |
| | (1-108)-anti- | SLKPEDTAVYYCNMHTFWGQGTQVTVSSRSGGGGSGGGGSG |
| | CD40(V12) VHH- | GGGSGGGGSGGGGSQVQLQESGGGLVQAGGSLRLSCAASG |
| | pCR3 | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQFSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTLEQVQLQESGGGLVQAGGSLRLSCAASGL |
| | | VFKRYSMNWYRQPPGQQRGLVASISDSGVSTNYADSVKGRFT |
| | | ISRDNAKNIGYLQMNSLKPEDTAVYYCNMHTFWGQGTQVTVS |
| | | S (SEQ ID NO: 40) |
| 41 | anti-CD40(V12) | QVQLQESGGGLVQAGGSLRLSCAASGLVFKRYSMNWYRQPP |
| | VHH(3x)-Flag-FAB1 | GQQRGLVASISDSGVSTNYADSVKGRFTISRDNAKNIGYLQMN |
| | (1-108)-anti- | SLKPEDTAVYYCNMHTFWGQGTQVTVSSRSGGGGSGGGGSG |
| | CD40(V12) VHH- | GGGSGGGGSGGGGSQVQLQESGGGLVQAGGSLRLSCAASG |
| | pCR3 | LVFKRYSMNWYRQPPGQQRGLVASISDSGVSTNYADSVKGRF |
| | | TISRDNAKNIGYLQMNSLKPEDTAVYYCNMHTFWGQGTQVTVS |
| | | SGGGGSGGGGSGGGGSGGGGSGGGGSQLQVQLQESGGGL |
| | | VQAGGSLRLSCAASGLVFKRYSMNWYRQPPGQQRGLVASISD |
| | | SGVSTNYADSVKGRFTISRDNAKNIGYLQMNSLKPEDTAVYYC |
| | | NMHTFWGQGTQVTVSSGSSSASTKGPSVFPLAPSSKSTSGG |
| | | TAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTLEQVQLQESGGGLVQAGGSLRLSCAASGLVFKRYSMNWY |
| | | RQPPGQQRGLVASISDSGVSTNYADSVKGRFTISRDNAKNIGY |
| | | LQMNSLKPEDTAVYYCNMHTFWGQGTQVTVSS (SEQ ID NO: |
| | | 41) |
| 42 | anti-CD40(V12)- | QVQLQESGGGLVQAGGSLRLSCAASGLVFKRYSMNWYRQPP |
| | VHH-Flag-TNC-anti- | GQQRGLVASISDSGVSTNYADSVKGRFTISRDNAKNIGYLQMN |
| | CD40(V12)-VHH- | SLKPEDTAVYYCNMHTFWGQGTQVTVSSGSDYKDDDDKDIAC |
| | pCR3 | GCAAAPDIKDLLSRLEELEGLVSSFREQGTGVEQVQLQESGGG |
| | | LVQAGGSLRLSCAASGLVFKRYSMNWYRQPPGQQRGLVASIS |
| | | DSGVSTNYADSVKGRFTISRDNAKNIGYLQMNSLKPEDTAVYY |
| | | CNMHTFWGQGTQVTVSS (SEQ ID NO: 42) |
| 43 | anti-CD40(15B8)- | DIVMTQSPLSLSVAPGQPASISCKSSQSLLESYGETYLYWYLQK |
| | Flag-VL-pCR3 | PGQPPQLLIYAVFKRFSGVPDRFSGSGSGTDFTLKISRVEAEDV |
| | | GVYYCMQSMQLPLTFGGGTKVEIKGSEIKRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC (SEQ ID NO: 43) |
| 44 | anti-CD40(15B8)- | QVQLQESGGGVVQPGRSLRLSCAASGFTFNNFGIHWVRQAPG |
| | Flag-VH-N297A- | KGLEWVAVISYDGSDKYYADSVKGRFTISRDNSKNTLNLQMNS |
| | TNC-PCR3 | LRAEDTAVYYCARDRRYYYHYYGMDVWGQGTMVTVSSRSSS |
| | | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVHN |
| | | AKTKPREEQYASTYRVVSVLTVLHQDWLNGKEYKCKVSNKAL |
| | | PAPIEKTISKAKGQPREPQVYTLPPSRDELTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGKLEDIACGCA |
| | | AAPDIKDLLSRLEELEGLVSSLREQGTG (SEQ ID NO: 44) |
| 45 | anti-CD40(15B8)- | QVQLQESGGGVVQPGRSLRLSCAASGFTFNNFGIHWVRQAPG |
| | Flag-VH- | KGLEWVAVISYDGSDKYYADSVKGRFTISRDNSKNTLNLQMNS |
| | IgG1(A + RGY)- | LRAEDTAVYYCARDRRYYYHYYGMDVWGQGTMVTVSSRSSS |
| | pCR3 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVHN |
| | | AKTKPREEQYASTYRVVSVLTVLHQDWLNGKEYKCKVSNKAL |
| | | PAPIEKTISKAKGQPRRPQVYTLPPSRDELTKNQVSLTCLVKG |
| | | FYPSDIAVEWESNGQPENNYKTTPPVLDSDGSFFLYSKLTVDK |
| | | SRWQQGNVFSCSVMHGALHNHYTQKYLSLSPGK (SEQ ID |
| | | NO: 45) |
| 46 | anti-CD40(15B8)- | QVQLQESGGGVVQPGRSLRLSCAASGFTFNNFGIHWVRQAPG |
| | Flag-VH-N297A- | KGLEWVAVISYDGSDKYYADSVKGRFTISRDNSKNTLNLQMNS |
| | scFv-anti- | LRAEDTAVYYCARDRRYYYHYYGMDVWGQGTMVTVSSRSSS |
| | CD40(15B8)-pCR3 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVHN |
| | | AKTKPREEQYASTYRVVSVLTVLHQDWLNGKEYKCKVSNKAL |
| | | PAPIEKTISKAKGQPREPQVYTLPPSRDELTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGKLEQVQLQE |
| | | SGGGVVQPGRSLRLSCAASGFTFNNFGIHWVRQAPGKGLEWV |
| | | AVISYDGSDKYYADSVKGRFTISRDNSKNTLNLQMNSLRAEDT |
| | | AVYYCARDRRYYYHYYGMDVWGQGTMVTVSSRSSTKGPKLE |
| | | EGEFSEAQLDIVMTQSPLSLSVAPGQPASISCKSSQSLLESYGE |
| | | TYLYWYLQKPGQPPQLLIYAVFKRFSGVPDRFSGSGSGTDFTL |
| | | KISRVEAEDVGVYYCMQSMQLPLTFGGGTKVEIK (SEQ ID NO: |
| | | 46) |
| 47 | anti-CD40(24-31)- | DIVMTQSPSFLSASVGDRVTITCKASQNVITAVAWYQQKPGKS |
| | Flag-VL-pCR3 | PKLLIYSASNRYTGVPDRFSGSGSGTDFTLTISSLQPEDFADYF |
| | | CQQYNSYPYTFGGGTKLEIKGSEIKRTVAAPSVFIFPPSDEQLK |
| | | SGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC (SEQ ID NO: 47) |
| 48 | anti-CD40(24-31)- | EVQLQESGPGLVKPSETLSLTCTVSGDSITNGFWIWIRKPPGNK |
| | Flag-VH-N297A- | LEYMGYISYSGSTYYNPSLKSRISISRDTSKNQFSLKLSSVTAAD |
| | TNC-pCR3 | TGVYYCACRSYGRTPYYFDFWGQGTTLTVSSRSSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTI |
| | | SKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGKLEDIACGCAAAPDIKD |
| | | LLSRLEELEGLVSSLREQGTG (SEQ ID NO: 48) |
| 49 | anti-CD40(24-31)- | EVQLQESGPGLVKPSETLSLTCTVSGDSITNGFWIWIRKPPGNK |
| | Flag-VH- | LEYMGYISYSGSTYYNPSLKSRISISRDTSKNQFSLKLSSVTAAD |
| | IgG1(A + RGY)- | TGVYYCACRSYGRTPYYFDFWGQGTTLTVSSRSSSASTKGPS |
| | pCR3 | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTI |
| | | SKAKGQPRRPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHGALHNHYTQKYLSLSPGK (SEQ ID NO: 49) |
| 50 | anti-CD40(24-31)- | EVQLQESGPGLVKPSETLSLTCTVSGDSITNGFWIWIRKPPGNK |
| | Flag-VH-N297A- | LEYMGYISYSGSTYYNPSLKSRISISRDTSKNQFSLKLSSVTAAD |
| | scFv-anti-CD40(24- | TGVYYCACRSYGRTPYYFDFWGQGTTLTVSSRSSSASTKGPS |
| | 31)-pCR3 | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTI |
| | | SKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGKLEEVQLQESGPGLVK |
| | | PSETLSLTCTVSGDSITNGFWIWIRKPPGNKLEYMGYISYSGST |
| | | YYNPSLKSRISISRDTSKNQFSLKLSSVTAADTGVYYCACRSYG |
| | | RTPYYFDFWGQGTTLTVSSRSSTKGPKLEEGEFSEAQLDIVMT |
| | | QSPSFLSASVGDRVTITCKASQNVITAVAWYQQKPGKSPKLLIY |
| | | SASNRYTGVPDRFSGSGSGTDFTLTISSLQPEDFADYFCQQYN |
| | | SYPYTFGGGTKLEIK (SEQ ID NO: 50) |
| 51 | anti-CD40(F5)-Flag- | DIQMTQSPSSVSGSVGDRVTITCRASQGISSWLAWYQQKPGK |
| | VL-PCR3 | APKLLIYAGSSLQSGVPSRFSGSGFGTDFTLTISSLQPEDFATY |
| | | YCQQASSFPRTFQGTKVEIKGSEIKRTVAAPSVFIFPPSDEQLK |
| | | SGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC (SEQ ID NO: 51) |
| 52 | anti-CD40(F5)-Flag- | EVQLLESGGGLVQPGGSLRLSCAASGFTFSSYAMSWVRQAPG |
| | VH-N297A-TNC- | KGLEWVSAISGSGGSTYYADSVKGRFTISRDNSKNTLYLQMNS |
| | pCR3 | LRAEDTAVYYCAKDGGYYGSGSYGYFDYWGQGTLVTVSSRSS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPGKLEDIAC |
| | | GCAAAPDIKDLLSRLEELEGLVSSLREQGTG (SEQ ID NO: 52) |
| 53 | anti-CD40(F5)-Flag- | EVQLLESGGGLVQPGGSLRLSCAASGFTFSSYAMSWVRQAPG |
| | VH-IgG1(A + RGY)- | KGLEWVSAISGSGGSTYYADSVKGRFTISRDNSKNTLYLQMNS |
| | pCR3 | LRAEDTAVYYCAKDGGYYGSGSYGYFDYWGQGTLVTVSSRSS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPRRPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHGALHNHYTQKYLSLSPGK (SEQ ID |
| | | NO: 53) |
| 54 | anti-CD40(F5)-Flag- | EVQLLESGGGLVQPGGSLRLSCAASGFTFSSYAMSWVRQAPG |
| | VH-N297A-scFv- | KGLEWVSAISGSGGSTYYADSVKGRFTISRDNSKNTLYLQMNS |
| | anti-CD40(F5)- | LRAEDTAVYYCAKDGGYYGSGSYGYFDYWGQGTLVTVSSRSS |
| | pCR3 | SASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPGKLEEVQL |
| | | LESGGGLVQPGGSLRLSCAASGFTFSSYAMSWVRQAPGKGLE |
| | | WVSAISGSGGSTYYADSVKGRFTISRDNSKNTLYLQMNSLRAE |
| | | DTAVYYCAKDGGYYGSGSYGYFDYWGQGTLVTVSSRSSTKG |
| | | PKLEEGEFSEAQLDIQMTQSPSSVSGSVGDRVTITCRASQGIS |
| | | SWLAWYQQKPGKAPKLLIYAGSSLQSGVPSRFSGSGFGTDFTL |
| | | TISSLQPEDFATYYCQQASSFPRTFQGTKVEIK (SEQ ID NO: |
| | | 54) |
| 55 | anti- | EVQLQESGGGLVQAGGSLRLSCVTSGSAFSSDTMGWFRQAP |
| | CD40(V15t)VHH- | GKQRELVASISSRGVREYADSVKGRFTISRDNAKNTVYLQMNS |
| | Flag-LC-pCR3 | LQPEDTAVYYCNRGALGLPGYRPYNNWGQGTQVTVSSGSEIK |
| | | RTVAAPSVFIFPPSDEQLKSGTASVVCLLNNFYPREAKVQWK |
| | | VDNALQSGNSQESVTEQDSKDSTYSLSSTLTLSKADYEKHKV |
| | | YACEVTHQGLSSPVTKSFNRGEC (SEQ ID NO: 55) |
| 56 | anti-CD40(V15t)- | EVQLQESGGGLVQAGGSLRLSCVTSGSAFSSDTMGWFRQAP |
| | VHH-Flag-N297A- | GKQRELVASISSRGVREYADSVKGRFTISRDNAKNTVYLQMNS |
| | pCR3 | LQPEDTAVYYCNRGALGLPGYRPYNNWGQGTQVTVSSGSSS |
| | | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVHN |
| | | AKTKPREEQYASTYRVVSVLTVLHQDWLNGKEYKCKVSNKAL |
| | | PAPIEKTISKAKGQPREPQVYTLPPSRDELTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGK (SEQ ID |
| | | NO: 56) |
| 57 | anti- | EVQLQESGGGLVQAGGSLRLSCVTSGSAFSSDTMGWFRQAP |
| | CD40(V15t)VHH(3x) | GKQRELVASISSRGVREYADSVKGRFTISRDNAKNTVYLQMNS |
| | -Fc(DANA)-Flag- | LQPEDTAVYYCNRGALGLPGYRPYNNWGQGTQVTVSSRSGG |
| | pCR3 | GGSGGGGGGGGSGGGGSGGGGSEVQLQESGGGLVQAGG |
| | | SLRLSCVTSGSAFSSDTMGWFRQAPGKQRELVASISSRGVRE |
| | | YADSVKGRFTISRDNAKNTVYLQMNSLQPEDTAVYYCNRGALG |
| | | LPGYRPYNNWGQGTQVTVSSGGGGSGGGGSGGGGSGGGG |
| | | SGGGGSQLEVQLQESGGGLVQAGGSLRLSCVTSGSAFSSDT |
| | | MGWFRQAPGKQRELVASISSRGVREYADSVKGRFTISRDNAK |
| | | NTVYLQMNSLQPEDTAVYYCNRGALGLPGYRPYNNWGQGTQ |
| | | VTVSSGSKTHTCPPCPAPELLGGPSVFLFPPKPKDTLMISRTP |
| | | EVTCVVVAVSHEDPEVKFNWYVDGVEVHNAKTKPREEQYAST |
| | | YRVVSVLTVLHQDWLNGKEYKCKVSNKALPAPIEKTISKAKG |
| | | QPREPQVYTLPPSRDELTKNQVSLTCLVKGFYPSDIAVEWESN |
| | | GQPENNYKTTPPVLDSDGSFFLYSKLTVDKSRWQQGNVFSCS |
| | | VMHEALHNHYTQKSLSLSPGKEFDYKDDDDKLE (SEQ ID NO: |
| | | 57) |
| 58 | anti- | EVQLQESGGGLVQAGGSLRLSCVTSGSAFSSDTMGWFRQAP |
| | CD40(V15t)(3x)- | GKQRELVASISSRGVREYADSVKGRFTISRDNAKNTVYLQMNS |
| | Flag-TNC-pCR3 | LQPEDTAVYYCNRGALGLPGYRPYNNWGQGTQVTVSSRSGG |
| | | GGSGGGGSGGGGSGGGGSGGGGSEVQLQESGGGLVQAGG |
| | | SLRLSCVTSGSAFSSDTMGWFRQAPGKQRELVASISSRGVRE |
| | | YADSVKGRFTISRDNAKNTVYLQMNSLQPEDTAVYYCNRGALG |
| | | LPGYRPYNNWGQGTQVTVSSGGGGSGGGGSGGGGSGGGG |
| | | SGGGGSQLEVQLQESGGGLVQAGGSLRLSCVTSGSAFSSDT |
| | | MGWFRQAPGKQRELVASISSRGVREYADSVKGRFTISRDNAK |
| | | NTVYLQMNSLQPEDTAVYYCNRGALGLPGYRPYNNWGQGTQ |
| | | VTVSSGSDYKDDDDKDIACGCAAAPDIKDLLSRLEELEGLVSSF |
| | | REQGTG (SEQ ID NO: 58) |

Legend:
XXX restriction site encoding 2 AAs linker sequence
XXX Tenascin C (TNC) trimerization domain (DIACGCAAAPDIKDLLSRLEELEGLVSSLREQGTG; SEQ ID NO: 60)
XXX internal flag tag (DYKDDDDK)
XXX VL (variable light) domain: IgG1(N297A), IgG1(A + RGY), Fc, Fc(DANA), FAB1(1-108)
XXX variable domain
XXX linker sequence

For each of the plasmids No. 1-5, 9, 13, 17, 21, 24, 36, 43, 47, 51, 55, and 56 the encoded amino acid sequence is as follows: The indicated amino acid sequence of SEQ ID NOs 1-6, 9, 13, 17, 21, 24, 36, 43, 47, 51, 55, and 56, respectively, is preceded by an N-terminal amino acid sequence consisting of (in an N to C-terminal order): a leader sequence (SED ID NO: 30: MNFGFSLIFLVLVLKGVQCEVKLVPR), a first restriction site encoding a 2 AAs linker sequence consisting of the amino acids QL, a flag tag (SEQ ID:59: DYKDDDDK), and a second restriction site encoding 2 AAs linker sequence consisting of the amino acids EL.

For each of the plasmids No. 6, 7, 8, 10, 11, 12, 14, 15, 16, 18, 19, 20, 44, 45, 46, 48, 49, 50, and 52-54, the encoded amino acid sequence is as follows: The indicated amino acid sequence of SEQ ID NOs 7, 8, 10, 11, 12, 14, 15, 16, 18, 19, 20, 44, 45, 46, 48, 49, 50, and 52-54, respectively, is preceded by an N-terminal amino acid sequence consisting of (in an N- to C-terminal order): a leader sequence (SEQ ID NO: 30: MNFGFSLIFLVLVLKGVQCEVKLVPR), a first restriction site encoding a 2 AAs linker sequence consisting of the amino acids QL, a flag tag (SEQ ID NO: 59: DYKDDDDK), and a second restriction site encoding 2 AAs linker sequence consisting of the amino acids EF.

For each of the plasmids No. 22, 23, 25, 26, and 37-41, the encoded amino acid sequence is as follows: The indicated amino acid sequence of SEQ ID NOs 22, 23, 25, 26, and 37-41, respectively, is preceded by an N-terminal amino acid sequence consisting of (in an N- to C-terminal order): a leader sequence (SEQ ID NO: 30: MNFGFSLIFLVLVLKGVQCEVKLVPR)), a first restriction site encoding a 2 AAs linker sequence consisting of the amino acids QL, a flag tag (SEQ ID NO: 61: DYKDDDDK), and a second restriction site encoding 2 AAs linker sequence consisting of the amino acids GT.

For the plasmid NOs 27, 31, 34 and 42, the encoded amino acid sequence is as follows: The indicated amino acid sequence of SEQ ID NO 27 and 42, respectively, is preceded by an N-terminal amino acid sequence consisting of (in an N- to C-terminal order): a leader sequence (SEQ ID NO: 30: MNFGFSLIFLVLVLKGVQCEVKLVPR), and a restriction site encoding a 2 AAs linker sequence consisting of the amino acids QL.

For each of the plasmids No. 28-29, 32, 33, 35, 57 and 58 the encoded amino acid sequence is as follows: The indicated amino acid sequence of SEQ ID NOs 28-29, respectively, is preceded by an N-terminal amino acid sequence consisting of (in an N- to C-terminal order): a leader sequence (SEQ ID NO: 30: MNFGFSLIFLVLVLKGVQCEVKLVPR), and a restriction site encoding a 2 AAs linker sequence consisting of the amino acids GT.

Note that in accordance with the invention, the above-indicated restriction site encoding a 2 AAs linker sequence are cloning-related amino acid linker sequences and may be independently removed, or they may be independently replaced by another linker sequence comprising 1-20 amino acids. Similarly, and in accordance with the invention, the above-indicated internal linker sequence STKGPKLEEGEFSEA in the amino acid sequence of SEQ ID NOs 8, 12, 16, 20, 45, 50, and 54, respectively, and the above-indicated internal linker sequence GGGGSGGGGSGGGGSGGGGSGGGGS in the amino acid sequence of SEQ ID NOs 22, 23, 25, 26, 28, 28, 32, 33, 35, 37, 38, 40, 41, 57, and 58, respectively, are internal linker sequences and may be independently removed, or they may be independently replaced by another linker sequence comprising 1-20 amino acids.

TABLE 2
CD40 agonists proteins.

| | | | binding |
|---|---|---|---|
| | | | site number |
| | | | (N-term + |
| No | Protein name | Plasmid(s) | C-term) |
| 1 | antiCD40(G28.5)-N297A-HC:TNC | 1 + 2 | 6 + 0 |
| 2 | antiCD40(G28.5)-IgG1(A + RGY) | 1 + 3 | 12 + 0 |
| 3 | antiCD40(G28.5)-N297a-HC:scFvG28.5 | 1 + 4 | 2 + 2 |
| 4 | antiCD40(Lob7.4)-N297A-HC:TNC | 5 + 6 | 6 + 0 |
| 5 | antiCD40(Lob7.4)-IgG1(A + RGY) | 5 + 7 | 12 + 0 |
| 6 | antiCD40(Lob7.4)-N297A-HC:scFvLob7.4 | 5 + 8 | 2 + 2 |
| 7 | antiCD40(Mita)-N297A-HC:TNC | 9 + 10 | 6 + 0 |
| 8 | antiCD40(Mita)-IgG1(A + RGY) | 9 + 11 | 12 + 0 |
| 9 | antiCD40(Mita)-N297A-HC:scFvMita | 9 + 12 | 2 + 2 |
| 10 | antiCD40(Apex)-N297A-HC:TNC | 13 + 14 | 6 + 0 |
| 11 | antiCD40(Apex)-IgG1(A + RGY) | 13 + 15 | 12 + 0 |
| 12 | antiCD40(Apex)-N297A-HC:scFvApex | 13 + 16 | 2 + 2 |
| 13 | antiCD40(Seli)-N297A-HC:TNC | 17 + 18 | 6 + 0 |
| 14 | antiCD40(Seli)-IgG1(A + RGY) | 17 + 19 | 12 + 0 |
| 15 | antiCD40(Seli)-N297A-HC:scFvSeli | 17 + 20 | 2 + 2 |
| 16 | LC:VHH(V12)-HC:VHH(V12)-N297A | 21 + 24 | 4 + 0 |
| 17 | LC:VHH(V12)-HC:2xVHH(V12)-N297A | 21 + 25 | 6 + 0 |
| 18 | LC:VHH(V12)-HC:3xVHH(V12)-N297A | 21 + 26 | 8 + 0 |
| 19 | LC:2xVHH(V12)-HC:VHH(V12)-N297A | 22 + 24 | 6 + 0 |
| 20 | LC:2xVHH(V12)-HC:2xVHH(V12)-N297A | 22 + 25 | 8 + 0 |
| 21 | LC:2xVHH(V12)-HC:3xVHH(V12)-N297A | 22 + 26 | 10 + 0 |
| 22 | LC:3xVHH(V12)-HC:VHH(V12)-N297A | 23 + 24 | 8 + 0 |
| 23 | LC:3xVHH(V12)-HC:2xVHH(V12)-N297A | 23 + 25 | 10 + 0 |
| 24 | LC:3xVHH(V12)-HC:3xVHH(V12)-N297A | 23 + 26 | 12 + 0 |
| 25 | VHH(V12)-TNC | 27 | 3 + 0 |
| 26 | 2xVHH(V12)-TNC | 28 | 6 + 0 |
| 27 | 3xVHH(V12)-TNC | 29 | 9 + 0 |
| 28 | VHH(V12)-Fc(DANA) | 31 | 2 + 0 |
| 29 | 2xVHH(V12)-Fc(DANA) | 32 | 4 + 0 |
| 30 | 3xVHH(V12)-Fc(DANA) | 33 | 6 + 0 |
| 31 | VHH(V12)-Fc(DANA)-VHH(V12) | 34 | 2 + 2 |
| 32 | 3xVHH(V12)-Fc(DANA)-VHH(V12) | 35 | 6 + 2 |
| 33 | LC:VHH(V12)-HC:VHH(V12)-Fab1 | 21 + 36 | 2 + 0 |
| 34 | LC:2xVHH(V12)-HC:VHH(V12)-Fab1 | 22 + 36 | 3 + 0 |
| 35 | LC:3xVHH(V12)-HC:VHH(V12)-Fab1 | 23 + 36 | 4 + 0 |
| 36 | LC:VHH(V12t)-HC:VHH(V12)-Fab1-HC:VHH(V12) | 21 + 39 | 2 + 1 |
| 37 | LC:2xVHH(V12)-HC:VHH(V12)-Fab1-HC:VHH(V12) | 22 + 39 | 3 + 1 |
| 38 | LC:3xVHH(V12)-HC:VHH(V12)-Fab1-HC:VHH(V12) | 23 + 39 | 4 + 1 |
| 39 | LC:VHH(V12)-HC:2xVHH(V12)-Fab1-HC:VHH(V12) | 21 + 40 | 3 + 1 |
| 40 | LC:2xVHH(V12)-HC:2xVHH(V12)-Fab1-HC:VHH(V12) | 22 + 40 | 4 + 1 |
| 41 | LC:3xVHH(V12)-HC:2xVHH(V12)-Fab1-HC:VHH(V12) | 23 + 40 | 5 + 1 |
| 42 | LC:VHH(V12)-HC:3xVHH(V12)-Fab1-HC:VHH(V12) | 21 + 41 | 4 + 1 |
| 43 | LC:2xVHH(V12)-HC:3xVHH(V12)-Fab1-HC:VHH(V12) | 22 + 41 | 5 + 1 |
| 44 | LC:3xVHH(V12)-HC:3xVHH(V12)-Fab1-HC:VHH(V12) | 23 + 41 | 6 + 1 |
| 45 | VHH(V12)-TNC-VHH(V12) | 42 | 3 + 3 |
| 46 | LC:VHH(irr)-HC:VHH(V12)-Fab1-HC:VHH(V12) | X + 39 | 1 + 1 |
| 47 | LC:VHH(irr)-HC:3xVHH(V12)-Fab1-HC:VHH(V12) | X + 41 | 3 + 1 |
| 48 | antiCD40(15B8)-N297A-HC:TNC | 43 + 44 | 6 + 0 |
| 49 | antiCD40(15B8)-IgG1(A + RGY) | 43 + 45 | 12 + 0 |
| 50 | antiCD40(15B8)-N297A-HC:scFv15B8 | 43 + 46 | 2 + 2 |
| 51 | antiCD40(24-31)-N297A-HC:TNC | 47 + 48 | 6 + 0 |
| 52 | antiCD40(24-31)-IgG1(A + RGY) | 47 + 49 | 12 + 0 |
| 53 | antiCD40(24-31)-N297A-HC:scFv24-31 | 47 + 50 | 2 + 2 |
| 54 | antiCD40(F5)-N297A-HC:TNC | 51 + 52 | 6 + 0 |
| 55 | antiCD40(F5)-IgG1(A + RGY) | 51 + 53 | 12 + 0 |
| 56 | antiCD40(F5)-N297A-HC:scFvF5 | 51 + 54 | 2 + 2 |
| 57 | LC:VHH(V15)-HC:VHH(V15)-N297A | 55 + 56 | 4 + 0 |
| 58 | 3xVHH(V15)-Fc(DANA) | 57 | 6 + 0 |
| 59 | 3xVHH(V15)-TNC | 58 | 9 + 0 |

Note that “X” can be any single-domain antibody (sdAb).

### Examples

The present invention is further illustrated by the following non-limiting examples.

### Material and Methods

### Cell Culture

HEK293, U20S, L929, A20J and HT1080 cells (ATCC, Rockville, MD, USA) as well as HT1080-CD40 transfectants (Wyzgol et al., 2009) were cultivated at 37° C. and 5% CO2 and were regularly split twice a week. HEK293, A20J and HT1080/HT1080-CD40 cells were cultivated in RPMI 1640 medium (Gibco) and U20S and L929 cells in DMEM with high glucose medium (Glutamax, Gibco). Culture media were supplemented with 10% fetal calf serum (FCS; Life technologies, Karlsruhe, Germany).

### Expression Plasmids

Standard cloning techniques, DNA amplicons and synthetic DNA fragments were used to generate pCR3-based expression plasmids encoding the proteins and antibody chains listed in Table 1. The antibody variants used in this study are listed in Table 2. AA sequences of building blocks of the proteins encoded b the plasmids of Table 1 are listed in Table 3.

TABLE 3
Amino acid sequences of building blocks of proteins encoded by the plasmids of Table 1.

| | | GenBank |
|---|---|---|
| | Amino acids | (accession) |
| Signal peptide | MNFGFSLIFLVLVLKGVQCEVKLVPR | |
| Flag tag | DYKDDDDK | |
| Tenascin-C | 110-139 | XP_025905028 |
| Restriction sites | QL encoded by Mfe1 (CAATTG) | |
| | EL encoded by EcoR1/Mfe1 (GAATTG) | |
| | GS encoded by BamH1 (GGATCC) | |
| | EF encoded by EcoR1 (GAATTC) | |
| | LE encoded by Xho1 (CTCGAG) | |
| Gaussia princeps luciferase | 18-185 | AAG54095 |
| (GpL) | | |
| Constant heavy chain of human | 145-476 | AAA02914.1 |
| IgG1 | | |
| Constant heavy chain of human | 145-476 with Fc mutation | AAA02914.1 |
| IgG1(A-RGY), with Fc mutation | N287A/E374R/E459G/S469Y | |
| N287A/E374R/E459G/S469Y | | |
| Constant heavy chain of human | 145-476 with Fc mutation N287A | AAA02914.1 |
| IgG1(N297A), with Fc mutation | | |
| N287A | | |
| Constant heavy chain of human | 145-476 | AAA02914.1 |
| IgG1, N to A mutation at position | | |
| 326 | | |
| Constant heavy chain of human | 1-325 | AAB59393.1 |
| IgG2 | | |
| Constant heavy chain of human | 1-326 | AAB59394.1 |
| IgG4 | | |
| Constant light chain | 105-214 | BAA97671.1 |
| Linker | GGGGSGGGGSGGGGSGGGGSGGGGS | |
| CD40(ed) | 1-192 | AAO43990.1 |
| CD40(ed)-CRD1-4 | 1-187 | AAO43990.1 |
| CD40(ed)-CRD1-3 | 1-144 | AAO43990.1 |
| CD40(ed)-CRD1-2 | 1-103 | AAO43990.1 |
| CD40(ed)-CDR1 | 1-60 | AAO43990.1 |
| VH and VL of G28.5 domain | VL: 148-259 VH: 21-130 (from scFv sequence) | AJ853736 |
| Selicrelumab | VL: 1-117, VH: 1-126 | KEGG drug entry |
| | | D11491 |
| APX005M | As indicated VL and VH in aa sequence for | WO 2014/070934 |
| | light and heavy chain R-8 | Al |
| ChiLob7.4 | VL: 1-117, VH: 1-122 | PDB entries |
| | | 6FAX_L (V L) and |
| | | 6FAX_H (VH) |
| Mitazalimab | VL: 1-110, VH: 1-119 | Thera-SAbDab¹ |
| Humanized 24-31 | VL: 1-110; VH: 1-120 | WO 01/94586 A2 |
| 15B8 | VL: 1-12, VH: 1-122 | WO 02/28904 A2 |
| F5-77 | VL: 1-106, VH_ 1-124 | US 2003/0059427 |
| | | A1 |
| V12t | 1-112 | De Weerdt et al., |
| | | 2021 |
| V15t | 1-121 | De Weerdt et al., |
| | | 2021 |

¹Therapeutic Structural Antibody Database

### Production and Purification of Recombinant Proteins

All recombinant proteins were produced by transfection of HEK293 cells with corresponding expression plasmids using polyethylenimine (PEI; Polyscience Inc., Warrington, USA) as described elsewhere in detail (Kums et al., 2017). For the generation of recombinant antibody variants comprising a light and a heavy chain, a 1:1 mixture of the corresponding expression plasmids was used. 5-7 days post transfection cell culture supernatants of transfected cells were collected and cleared from cell debris by centrifugation for 10 min (5000 g). The concentration of the recombinant proteins was estimated by western blot analysis of the supernatants along with Flag-tagged protein standards of known concentrations using anti-Flag antibody M2 (Sigma-Aldrich, Saint Louis, USA) and goat anti-mouse-IgG1 IRDye 800CW antibody (Licor, Lincoln, USA).

### Purification of Antibody Variants by Affinity Chromatography

To purify Flag-tagged antibody variants, affinity chromatography with anti-Flag M2 agarose and 3×Flag peptide (both Sigma-Aldrich, Steinheim, Germany) was performed as described by the manufacturer. After elution, purity of the proteins were analysed by SDS-PAGE and silver staining with the Pierce Silver Stain Kit (Thermo Fisher Scientific, MA, USA) according to the protocol of the manufacturer. The concentrations of the purified proteins were estimated by comparison with the proteins of known concentration of the Amersham LMW Calibration Kit for SDS Electrophoresis (GE Healthcare UK Limited, Little Chalfont, UK).

### Co-Culture, ELISA and ECL Western Blot

To evaluate activation of CD40, CD40-responsive cells (HT1080-CD40 or U20S) were grown over night on flat bottom cell culture plate. In parallel, HEK293 cells were transfected with expression plasmids encoding FcγRIIB or memCD40L or, as a negative control, empty vector using the PEI method (see above). Next day, the CD40 responsive cells were mixed (1:1) with the murine FcγRIIb and EV transfectants and stimulated the resulting co-cultures with the anti-CD40 antibody construct of interest. For analysis of CD40-induced IL8 production co-culture experiments were performed in 96 well plates (2-104+2-104 cells per well). After overnight challenge with the antibody constructs, supernatants were analysed for IL-8 content by enzyme-linked immunosorbent assay (ELISA; BD Biosciences, San Diego, USA) according to the manufacturers protocol. Co-cultures with memCD40L expressing transfectants served to define the maximal CD40-induced IL8 response. To evaluate CD40-mediated activation, p100 processing and TRAF1 induction were analysed by western blotting in U20S cells. For western blot experiments co-culture experiments were performed in 6 well plates (1-106+1106). After overnight stimulation, co-cultures were collected ice cold PBS by scraping with a rubber policeman. Cells were then washed twice with fresh ice cold PBS, pelleted and resuspended in Laemmli buffer. Samples were sonicated for 25 seconds at 100% amplitude with a sonication probe (UP100H Ultrasonic Processor, Hielscher, Germany), heated at 95° C. for 5 min and subjected to SDS-PAGE separation. After transfer of proteins to a nitrocellulose membrane western blot analysis was performed with an anti-p100/p52 (Millipore), anti-TRAF1 (Cell Signaling Technology Beverly, MA, USA), anti-R-actin (Sigma Aldrich) and horseradish peroxidase (HRP)-conjugated polyclonal rabbit anti-mouse antibody (Dako, Glostrup, Denmark) or HRP coupled anti-rabbit antibody (Cell Signaling Technology Beverly, MA, USA). Finally, membranes were developed by chemiluminscent western blot detection.

### Binding Studies

For binding studies 2-104 cells/well were cultured overnight in flat clear bottom black cell culture plates (Greiner Bio-One).

To analyse the inhibitory effect of anti-CD40 antibodies on CD40L binding, HT1080-CD40 cells were incubated with the anti-CD40 antibodies at 37° C. for 30 minutes in triplicates and then supplemented with GpL-TNC-CD40L (100 ng/ml). After one additional hour, cells were washed five times with ice cold PBS and cell-associated luciferase activity was measured.

To determine the specific binding of GpL-linked anti-CD40 antibodies, the latter were added pairwise with increasing concentrations to HT1080 (unspecific binding) and HT1080-CD40 (total binding) cells. After one hour at 37° C., cells were washed five times with fresh ice cold PBS and finally GpL activity was measured. Specific binding was calculated by subtraction of the unspecific binding values from the corresponding total binding values.

To determine the binding of GpL-tagged deletion mutants of the ectodomain of CD40 to the various anti-CD40 antibodies, black high binding 96-well plates (Greiner Bio-One) were coated with 1 μg/ml protein G overnight. After blocking remaining free binding sites with 10% FCS in PBS and a washing step with ice cold PBS, anti-CD40 antibodies (1 pg/ml) were added for 30 minutes in medium. After removal of free antibodies by two washing steps with ice cold PBS the GpL-linked CD40 deletion mutants were added for 1 hour at 37° C. Cells were then washed five times with ice cold PBS and luminescence was determined. GpL activities were measured by adding 25 μl of 1.5 μM coelenterazin substrate in PBS to the cells.

### Example 1: FcγR-Bound Anti-CD40-Antibodies Uniformly Display Strong Agonism Irrespective of Isoform and Idiotype

In view of the complex and sometimes even contradictory findings on the FcγR-dependency of the agonism of CD40 antibodies, a panel of anti-CD40 antibodies with widely differing properties was initially defined. This panel included the well-established and long known anti-CD40 antibody G28.5 (Clark et al., 1988) as well as several anti-CD40 antibodies currently being investigated in clinical trials and/or disclosed in patents (CP-870,893/Selicrelumab, Vonderheide et al., 2007; APX005M/Sotigalimab, Filbert et al., 2021; ADC-1013/Mitazalimab, Mangsbo et al., 2015; ChiLob7.4, Yu et al., 2018). All antibodies of the panel recognize the human CD40 molecule, but differ in their effect on CD40L binding. IgG1 variants of ADC-1013, APX005M and G28.5 significantly inhibited binding of a GpL fusion protein of soluble CD40L (GpL-CD40L) to CD40 expressing cells while CP-870,893 and ChiLob7.4 did not affect GpL-CD40L binding to CD40 (FIG. 1A). In line with this, the latter two antibodies had no effect on memCD40L-induced production of IL8, an easily measurable chemokine whose expression is dominantly regulated by the classical NFκB signaling pathway and which is thus efficiently induced by activation of CD40 (FIG. 1B). In contrast, ADC-1013, APX005M and G28.5 significantly inhibited this CD40 response (FIG. 1B). The antibodies of the panel furthermore differed in CD40 affinity and with regard to the region in the extracellular domain of CD40 with which they interact (FIG. 1C-E).

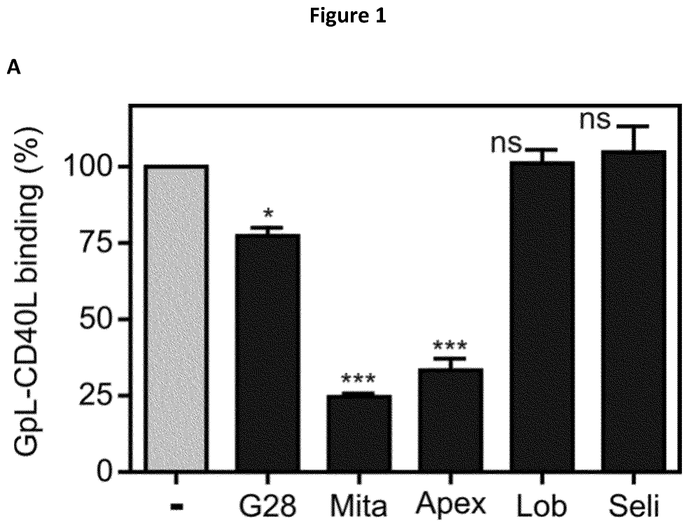

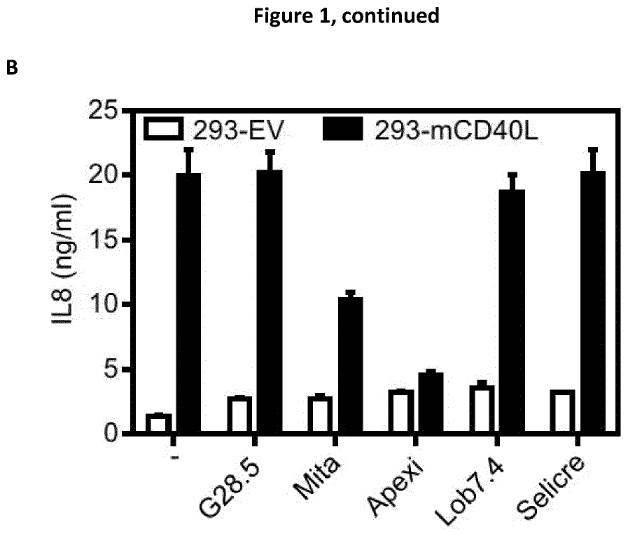

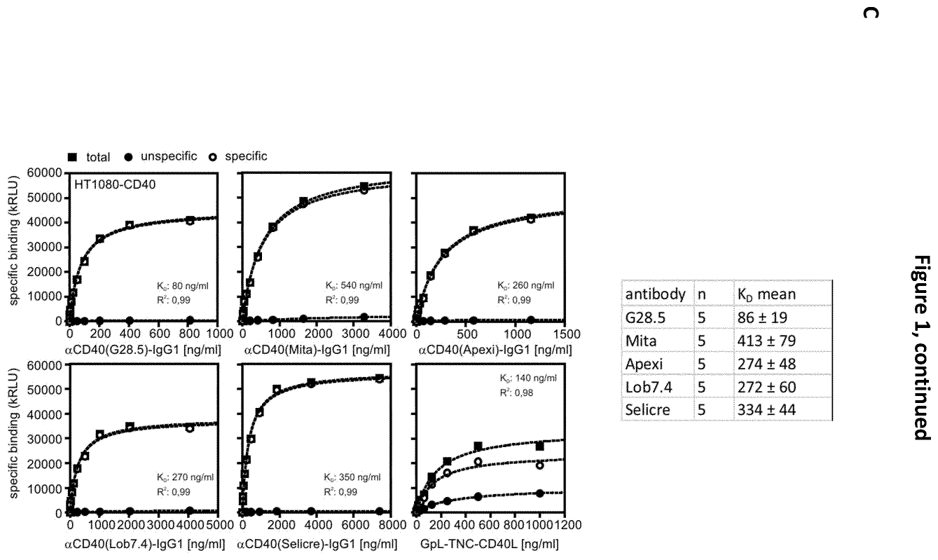

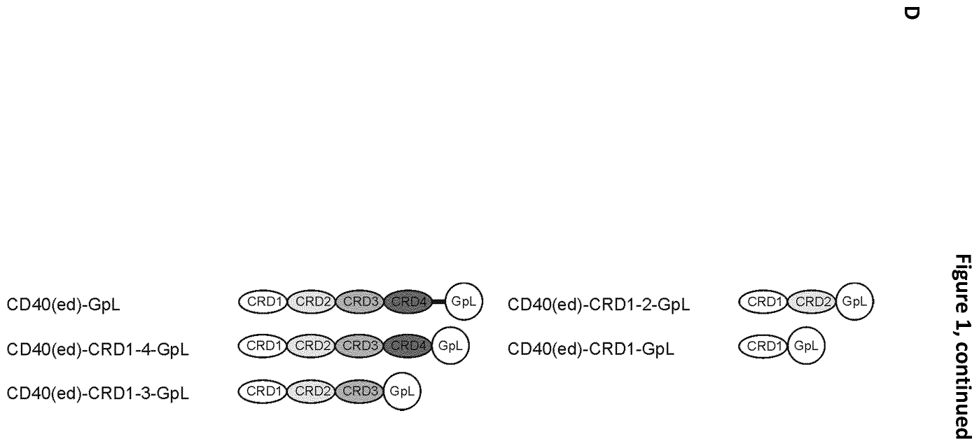

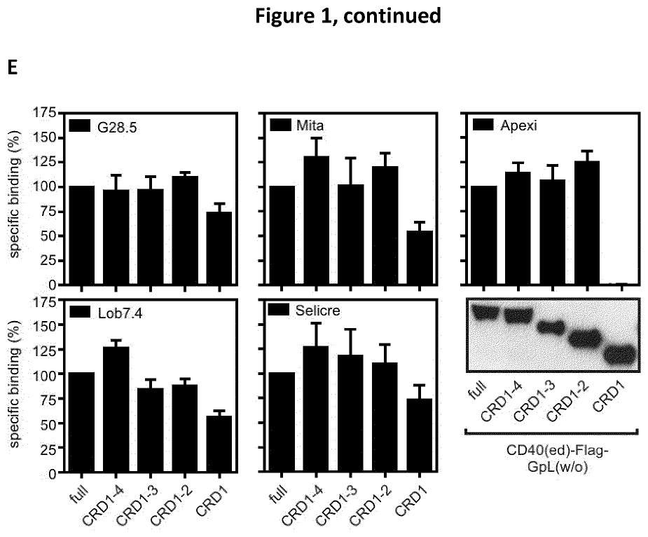

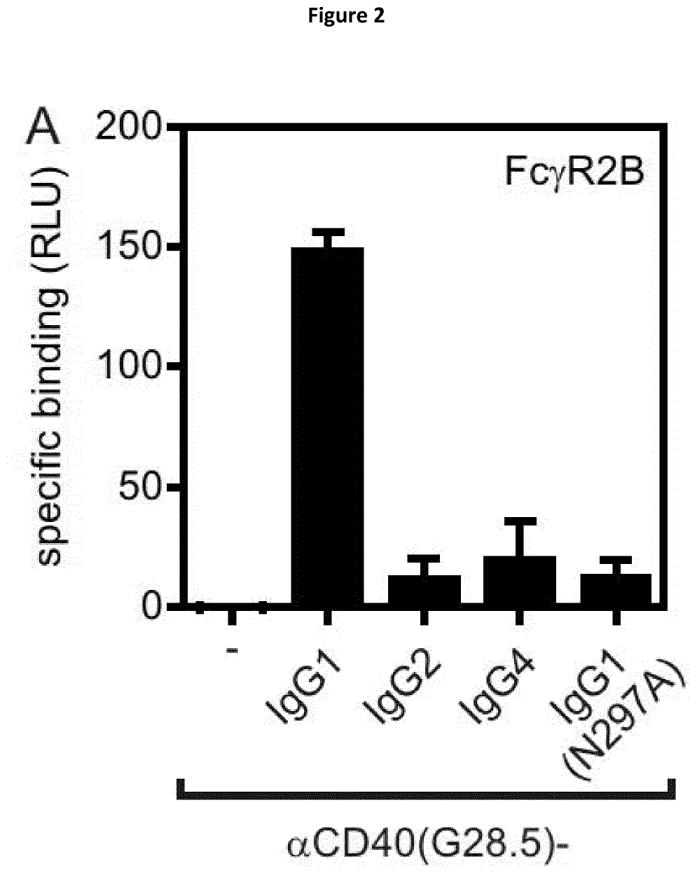

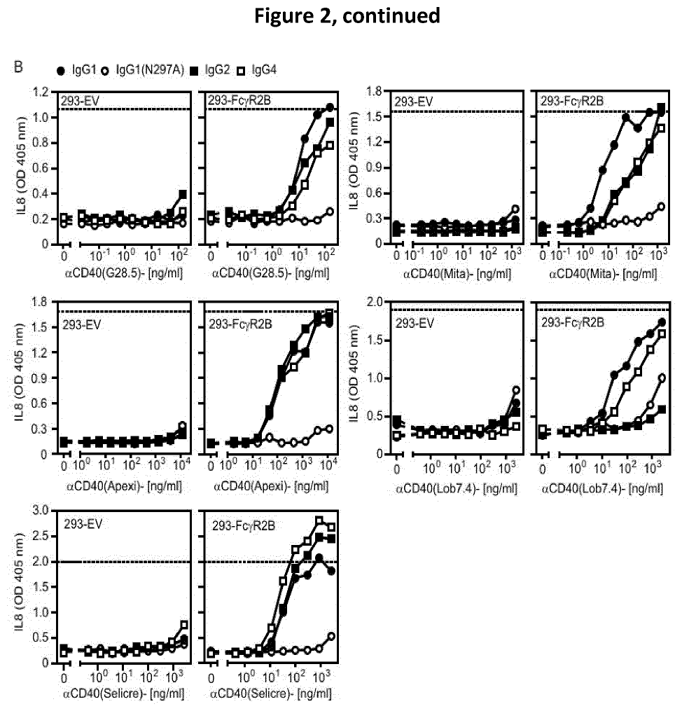

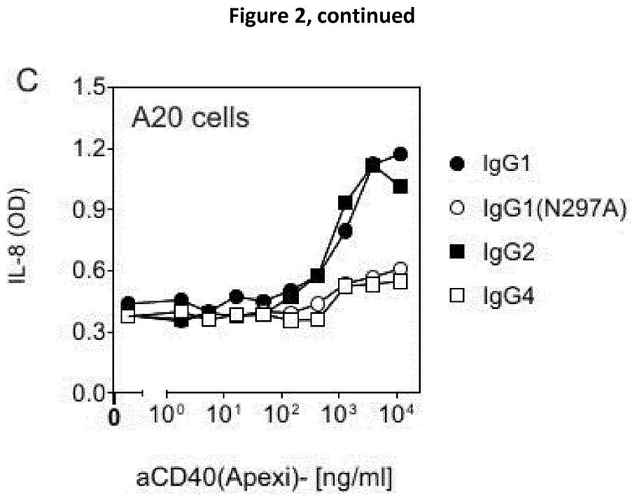

The anti-CD40 antibodies which are currently in clinical development are IgG1, IgG2 or IgG4 antibodies. The different in vitro agonism of these antibodies as well as their different in vivo tolerability has been attributed, at least in part, to isotype differences (Vonderheide and Glennie 2013; Vonderheide 2020). the anti-CD40 antibodies of the panel was therefore investigated not only in their original isotype, but also in the other two of the three isotypes mentioned. In addition, IgG1 variants (IgG1(N297A)) of the antibodies carrying a point mutation (N297A) was also analyzed, which strongly reduce binding FcγRI and abrogates binding to the remaining FcγRs (Wang et al., 2018). First, the ability of the conventional IgG variants of the anti-CD40 antibody panel to induce FcγR-dependent CD40 activation was evaluated. For this purpose, a simple in vitro system, in which CD40-expressing responder cells secreting large amounts of IL8 after CD40 stimulation (U2OS or HT1080-CD40 transfectants) were co-cultivated with HEK293 transfectants cells was used. The latter express little IL8 and were transiently transfected with the murine FcγRIIb capable of binding IgG1, IgG2 and weakly IgG4 (FIG. 2A), or with an empty vector. The use of FcγR transfectants had three advantages over the use of cells with endogenous FcγR expression. First, it was possible to analyze one type of FcγR without the possible interference by other FcγRs types. Second, there was a perfect negative control namely EV transfected HEK293 cells and third the high expression levels reached by transient transfection minimized the risk that CD40 engagement by FcγR-bound anti-CD40 antibodies is underestimated simply by the fact that the number of CD40 molecules in the system exceeds the number of FcγRs available for anti-CD40 presentation. All wild-type anti-CD40 IgG variants with the exception of those of the antibody Lob7.4, efficiently stimulated the production of IL8 in co-cultures with HEK293 transfectants expressing murine FcγRIIb reaching a similar or almost the same maximum response as obtained with memCD40L transfectants (FIG. 2B, lower panel). The agonistic activity of the IgG1 and IgG4 Lob7.4 variants was still significantly increased in co-cultures with FcγRIIb expressing HEK293 cells but not fully reached the level of memCD40L. In striking contrast, in co-cultures with HEK293 empty vector control transfectants, no or only a significantly weaker IL8 induction at high concentrations was found. As expected, the anti-CD40 IgG1(N297A) mutants of the various antibodies that do not bind to FcγRIIb showed little or no agonism both in co-cultures with FcγRIIb-transfected HEK293 cells (FIG. 2B, lower panel) as well as in co-cultures with control transfectants (FIG. 2B, upper panel). Principally similar results were obtained using cocultures with CD40 responder cells and murine A20 cells expressing FcγRs endogenously (FIG. 2C). A similar response pattern as for anti-CD40-induced IL8 production was also observed when CD40-mediated activation of the alternative NFκB signaling pathway was investigated. The alternative NFκB pathway essentially activates a different group of NFκB transcription factors than the classic signaling pathway and uses for this a distinct signaling pathway (Hayden and Gosh, 2012). Activation of the alternative NFκB pathway is characterized by the processing of a precursor protein termed p100 to the NFκB transcription factor subunit p52. In co-cultures with FcγRIIb-expressing HEK293 transfectants and U2OS cells, all anti-CD40 antibodies were able to efficiently process p100 to p52 and to upregulate expression of the TRAF1 protein, which is induced by both NFκB signaling pathways (FIG. 3). In contrast, in co-cultures with HEK293 control transfectants, there was only little or no evidence for activation of the alternative NFκB signaling pathway. Together, these results demonstrated that the agonism of plasma membrane presented anti-CD40 antibody-FcγR complexes is regularly much higher than that of the anti-CD40 IgG antibodies, irrespective of the epitope recognized by a concrete anti-CD40 antibody. Moreover, since the maximum IL8 induction achievable by FcγR-bound anti-CD40 antibodies is comparable to the IL8 production induced by membrane CD40L, it appears that FcγR-bound anti-CD40 antibodies, but not “free” anti-CD40 antibodies are able to stimulate the maximum possible CD40 activity.

### Example 2: Genetically Engineered Anti-CD40-Antibody Oligomers are Strong Agonists

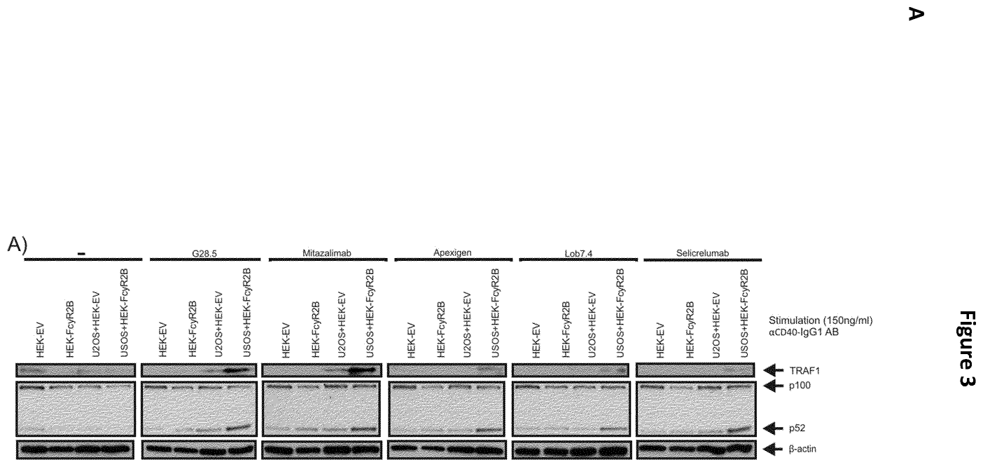

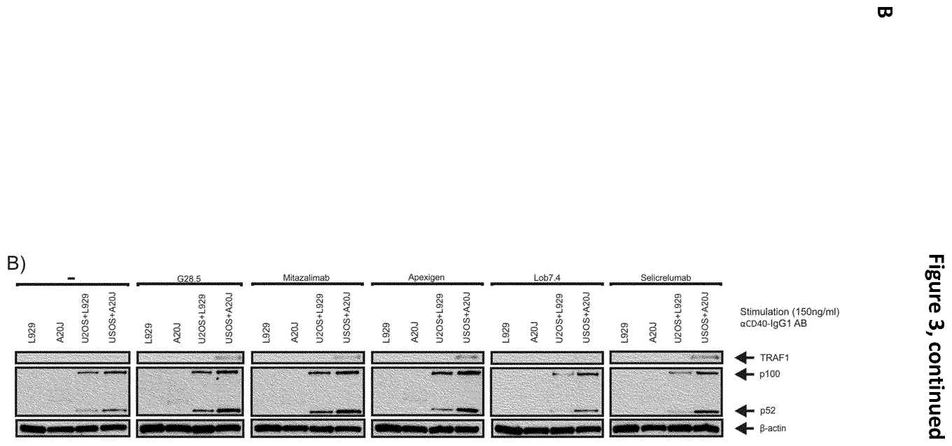

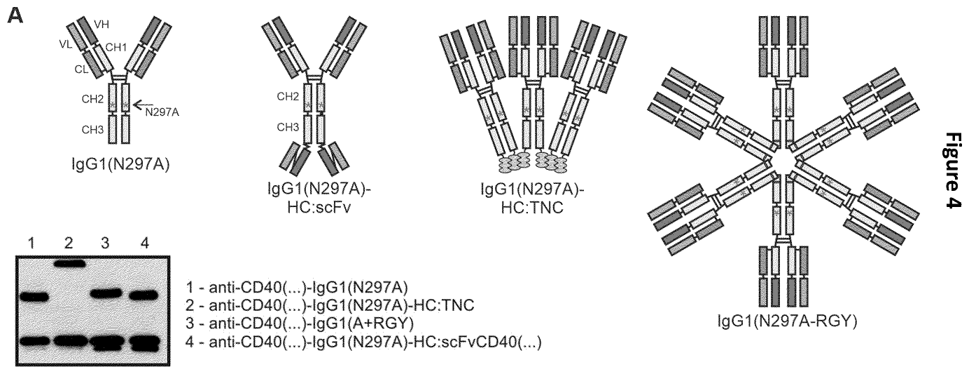

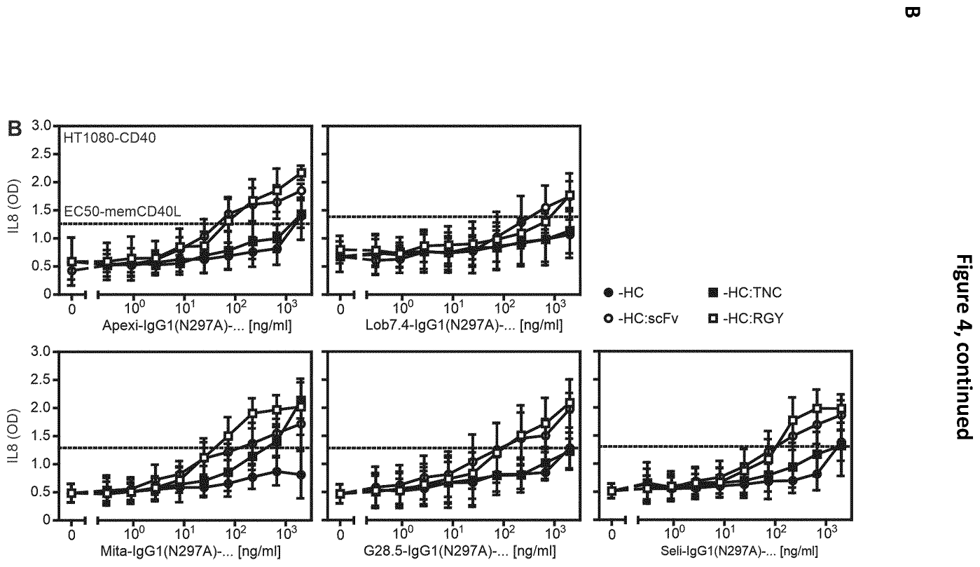

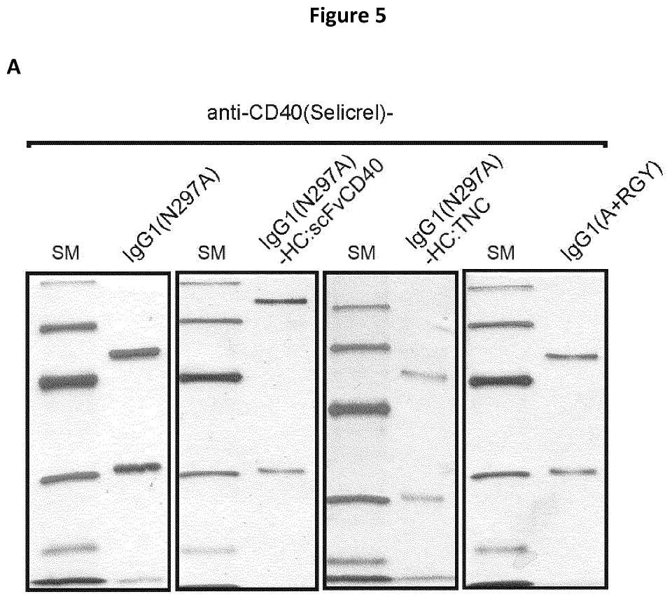

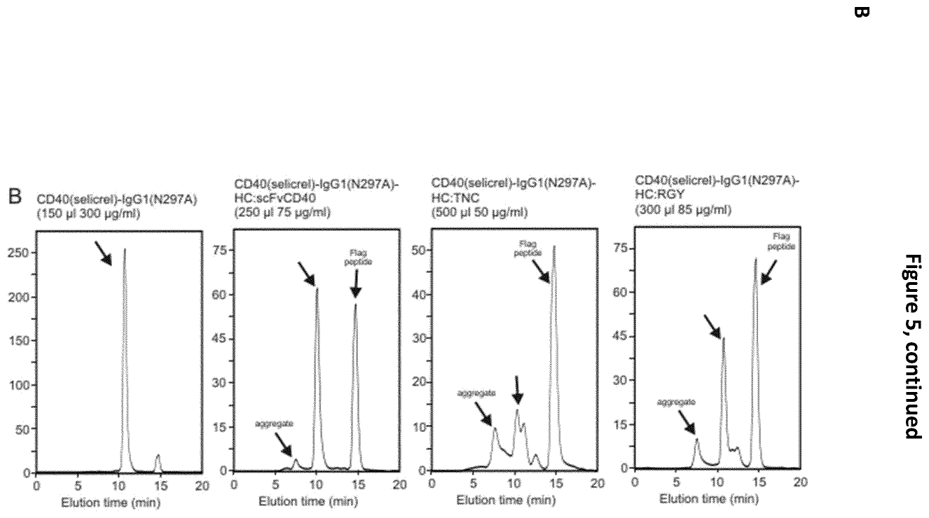

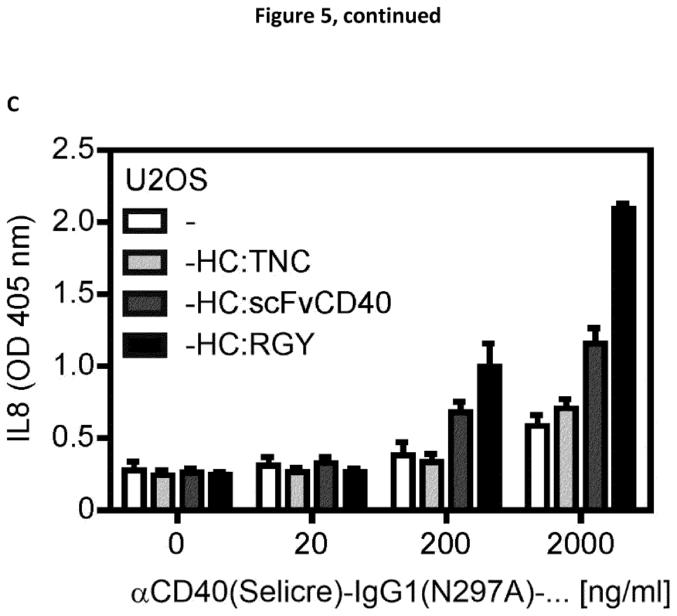

Since it is well-established that secondary crosslinking of anti-TNFR-antibodies can enhance their agonistic activity (Wajant 2015), it was investigated in the following whether the defined oligomerization of anti-CD40 antibodies by genetic engineering results in agonistic molecules. For this purpose, trimeric, thus hexavalent, and hexameric, thus dodecavalent variants of all IgG1(N297A) antibodies of the anti-CD40 antibody panel were generated. The hexavalent antibody variants were obtained by genetic fusion of the trimerization domain of the tenascin-C molecule, comprising about 30 amino acids, to the C terminus of the heavy chain of the IgG1(N297A) molecules (FIG. 4A). The dodecavalent antibody variants were obtained by introducing point mutations into the IgG1(N297A) heavy chain (Diebolder et al., 2014), which promote the assembly of hexameric IgG1 molecules (FIG. 4A). The various anti-CD40-IgG1(N297A)-TNC variants induced at concentrations between 0.2 and 2 μg/ml the half maximal IL8 response of the benchmark memCD40L expressing cells (FIG. 3B). The anti-CD40-IgG1(N297A-RGY) variants also acted as efficient CD40 agonists and were even regularly somewhat more efficient in CD40-dependent IL8 induction than the anti-CD40-IgG1(N297A)-TNC variants (FIG. 4B). In fact, with exception of the Lob7.4-based variant all anti-CD40-IgG1(N297A-RGY) constructs induced half maximal CD40-dependent IL8 production at concentrations below 100 ng/ml (FIG. 4B). Tetravalent variants of the anti-CD40-IgG1(N297A) antibodies which were obtained by fusing scFv domains generated from the variable domains of the antibodies to the C-termini of the antibody heavy chains (FIG. 4B) were also examined. These IgG1(N297A)-HC:scFv variants also showed a strong agonistic effect for all CD40 antibodies and were comparable active as the anti-CD40-IgG1(N297A-RGY) variants. The data shown so far were obtained with cell culture supernatants containing the anti-CD40 variant of interest. To verify that i) the agonistic activities observed are indeed related to oligomerized antibody molecules and that ii) the anti-CD40 fusion proteins maintain integrity, the CP-870,893 variants was exemplarily purified by gravity flow affinity chromatography on an anti-Flag antibody M2 (FIG. 5A). In fact, gel filtration analysis revealed high molecular weight peaks corresponding to the oligomerized antibody molecules but in the case of CP-870,893-IgG1(N297A-RGY and CP-870,893-IgG1(N297A)-TNC there were also peaks corresponding to the parental non-aggregated antibody molecules (FIG. 5B). Most important the purified protein samples were still efficient CD40 agonists (FIG. 5C).

### Example 3: Genetically Engineered Oligovalent CD40-Specific Single Domain Antibodies are Strong Agonists

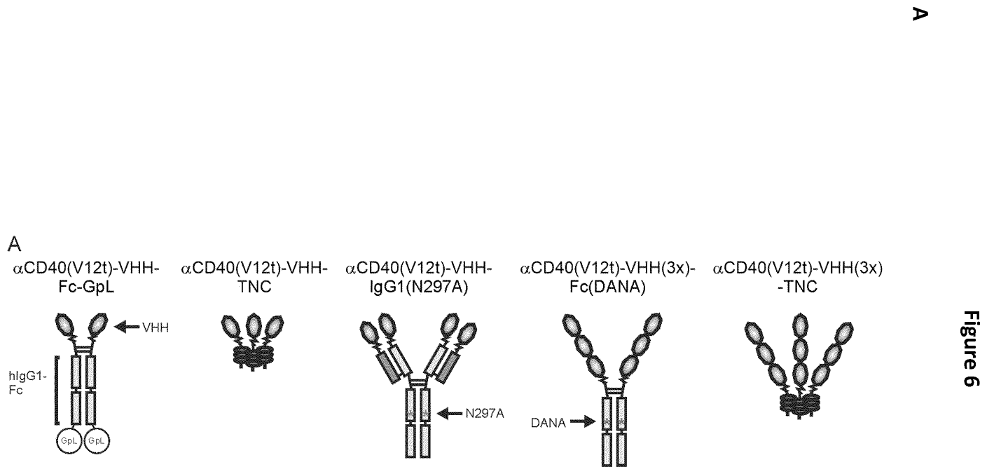

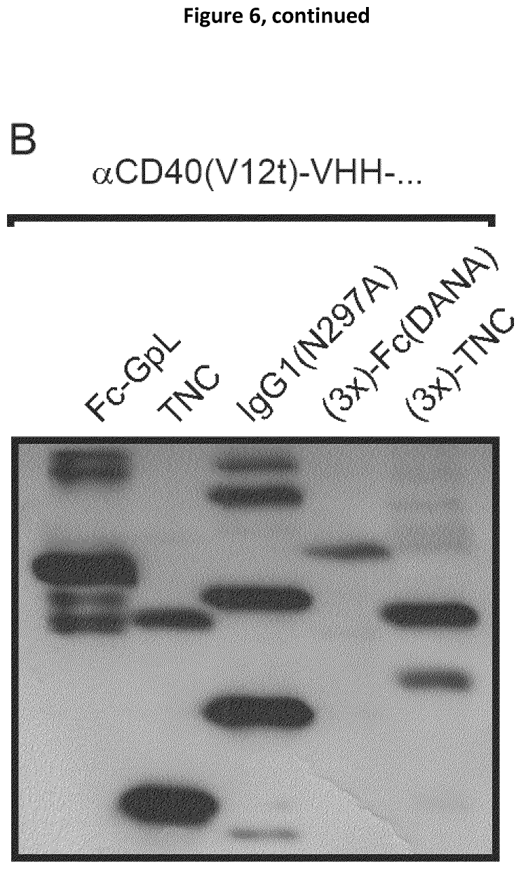

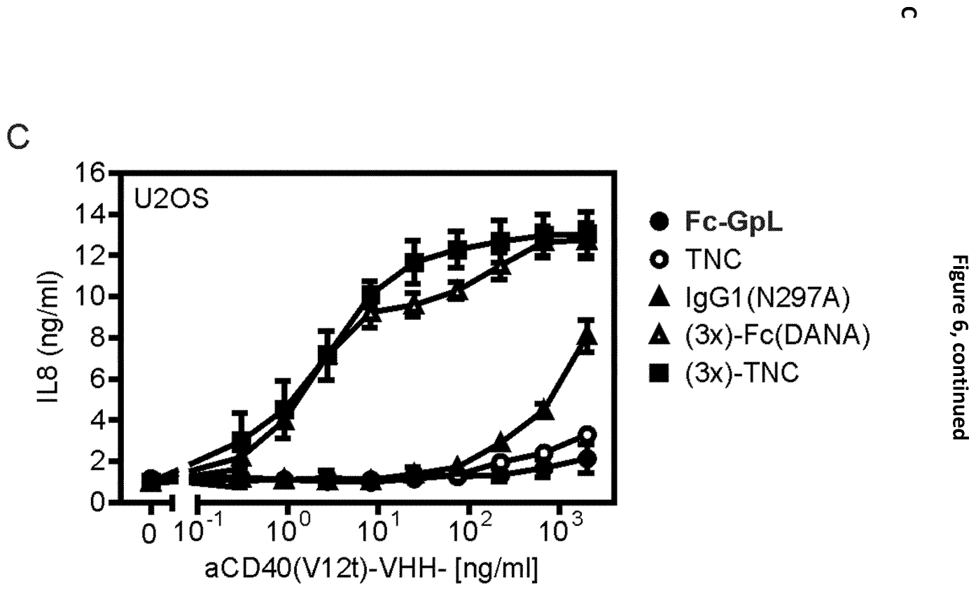

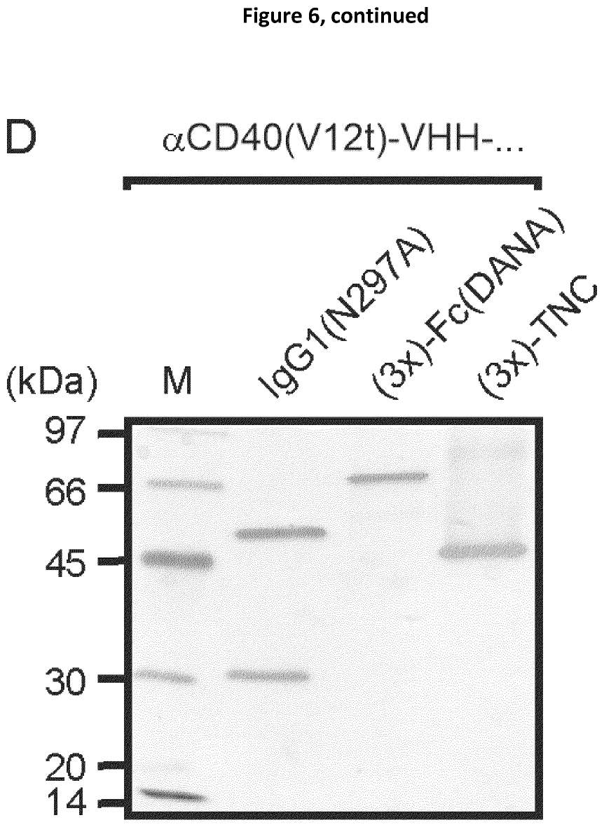

Similar results as with the oligomerized conventional antibodies were obtained when using human CD40-specific “single domain antibodies (sdAbs)” (de Weerdt et al., 2021) and oligomerizing protein scaffolds as building blocks (FIG. 6A,B). Like conventional bivalent anti-CD40 IgG antibodies, bivalent sdAb-Fc and trivalent sdAb-TNC fusion proteins proved to be poorly active/inactive (FIG. 6C). However, when three sdAb domains were cloned sequentially to the Fc domain of human IgG1 or to the trimerization domain of TNC, hexavalent and nonavalent molecules were obtained which show strong CD40-stimulating activity (FIG. 6C). A Tetravalent sdAb variants which were obtained by replacing the variable domains of an IgG1 molecule with a CD40-specific sdAb domain showed some activity but was by far less active compared to the hexa- and nonavalent variants. Gel filtration analysis of purified proteins revealed no aggregates for the tetra- and nonavalent variants but some aggregates for the hexavalent molecule.

### Example 4: Genetically Engineered Oligovalent CD40-Specific Variants Stimulate Maturation of Immature Human Dendritic Cells

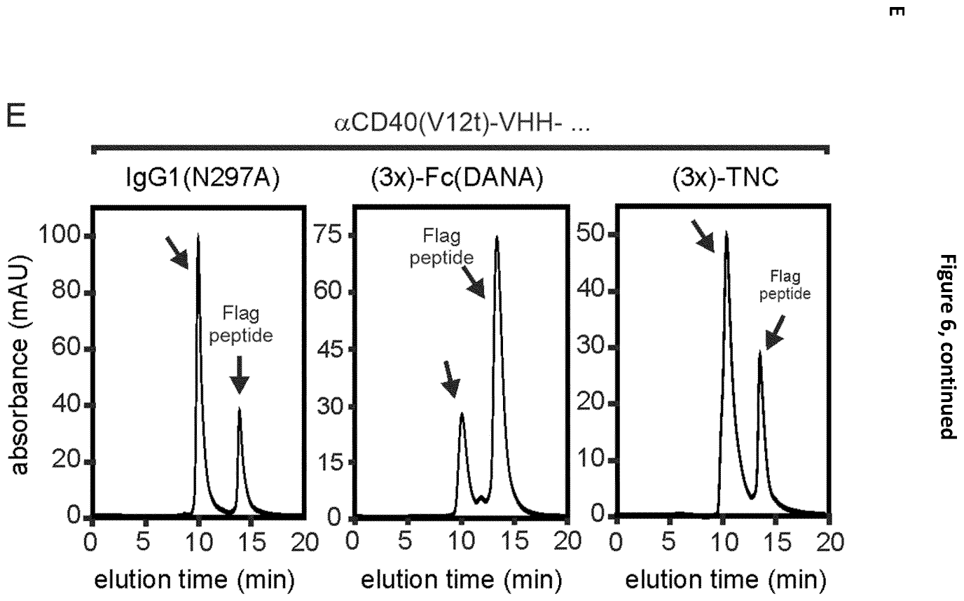

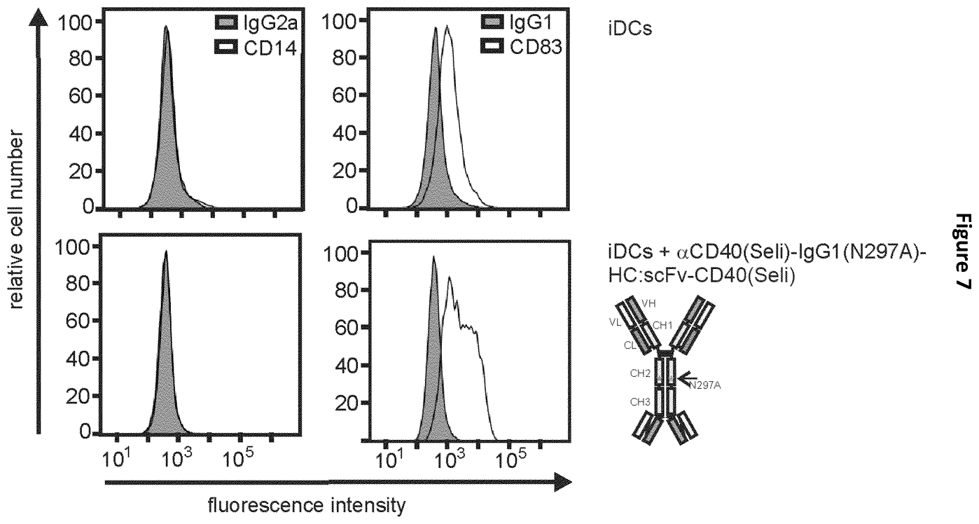

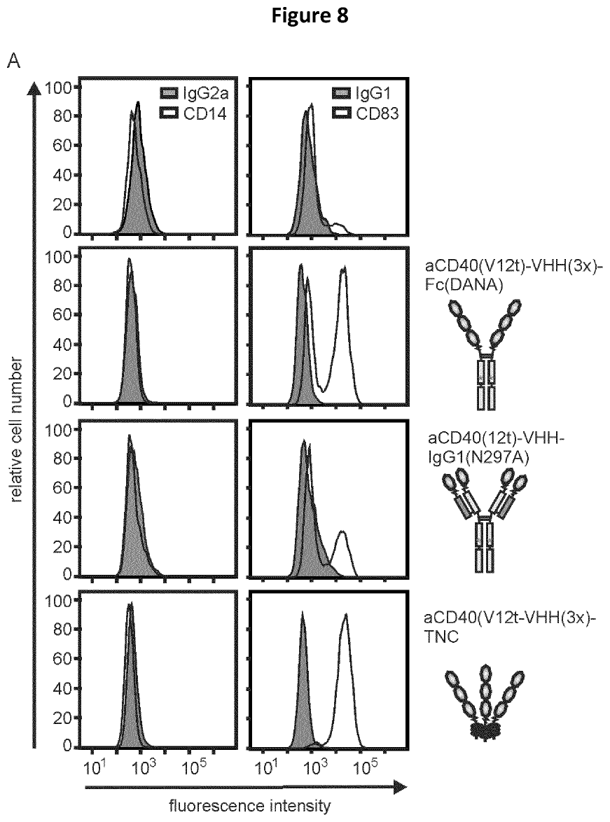

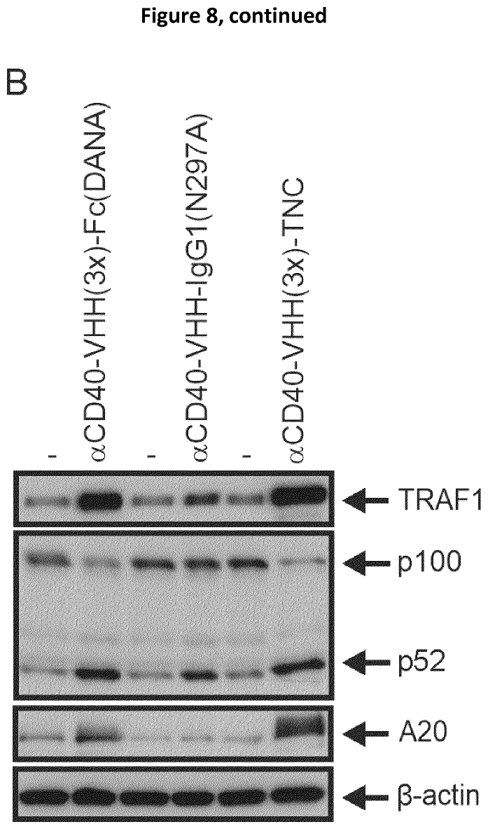

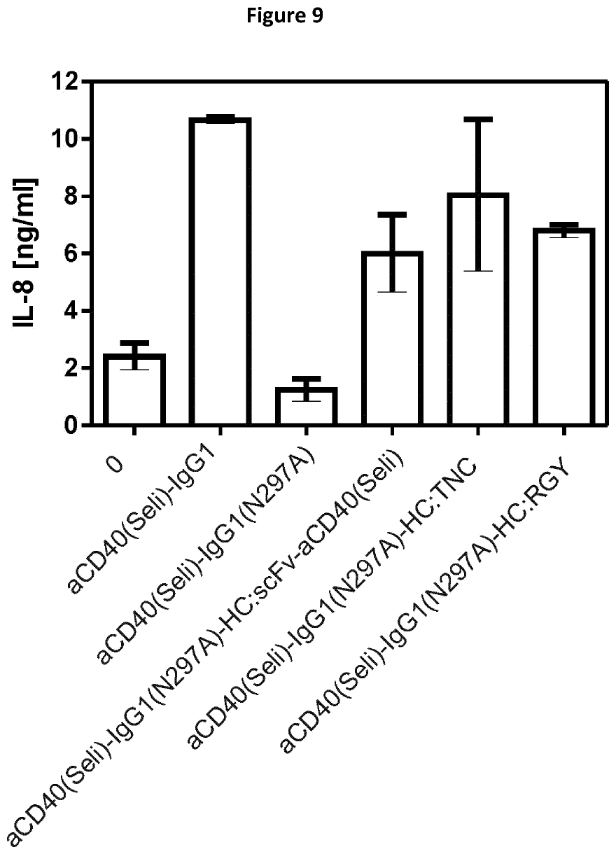

Immature monocyte-derived dendritic cells (iDCs) were generated by cultivation of monocytes for 7 days with GM-CSF/IL4. When iDCs were treated with the oligovalent CD40-specific variants described maturation of iDCs became evident by upregulation of CD83 measured by flow cytometry or by increased production of 1L8 and A20 measured by ELISA and Western blotting (FIGS. 7-10).

### Example 5: Number but Also Position of CD40-Binding Domains Determines Agonistic Activity

Various sdAb constructs with 2, 3, 4, 6, 8, 9, 10 or 12 parallel organized CD40 binding domains were obtained by fusion of one or two or three sdAb domains to the N-terminus of the human IgG1 Fc domain (N297A) version or by replacing the variable domains of an IgG1 antibody (again N297 version) or an Fab1.

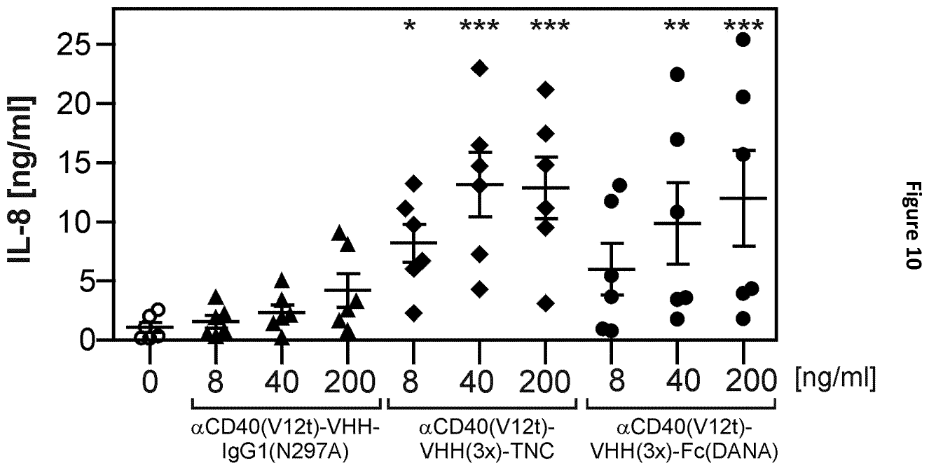

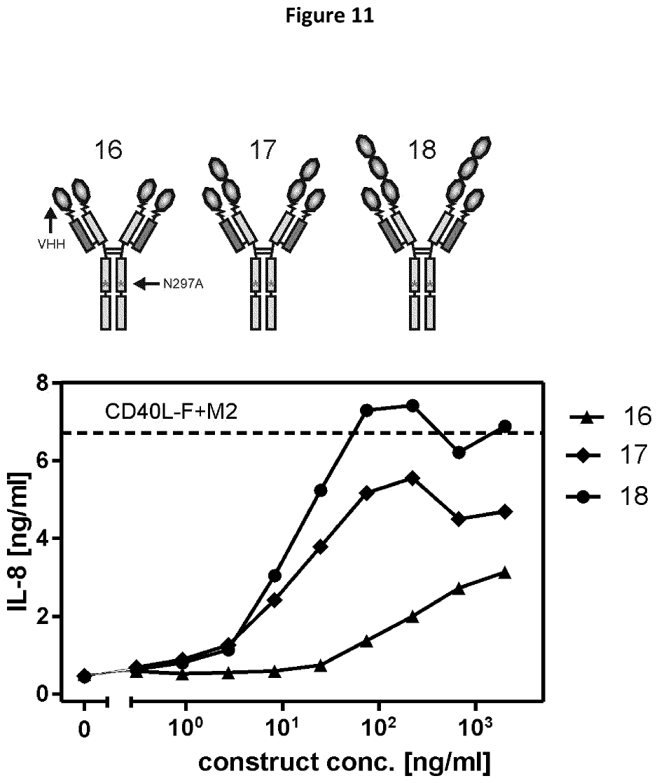

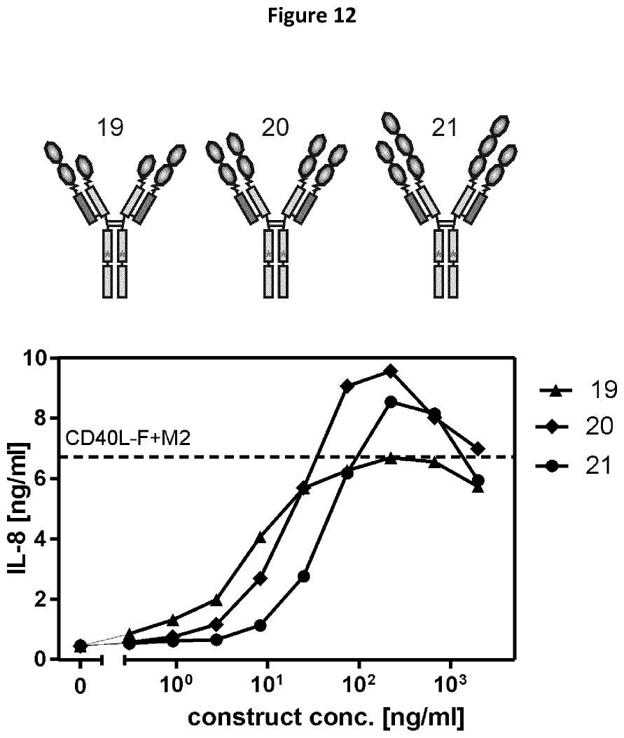

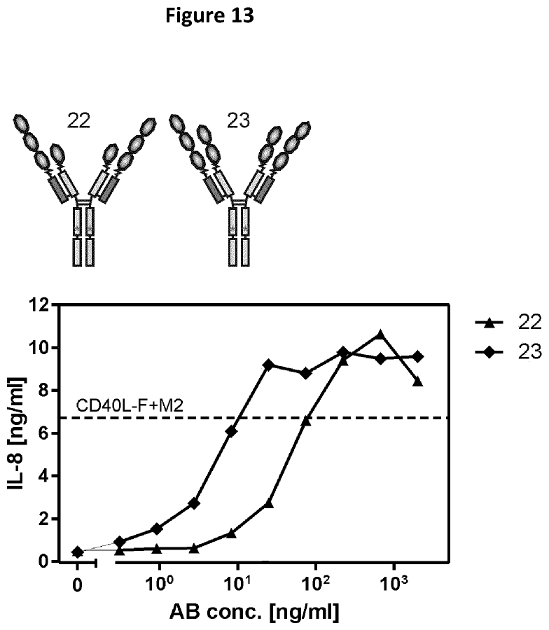

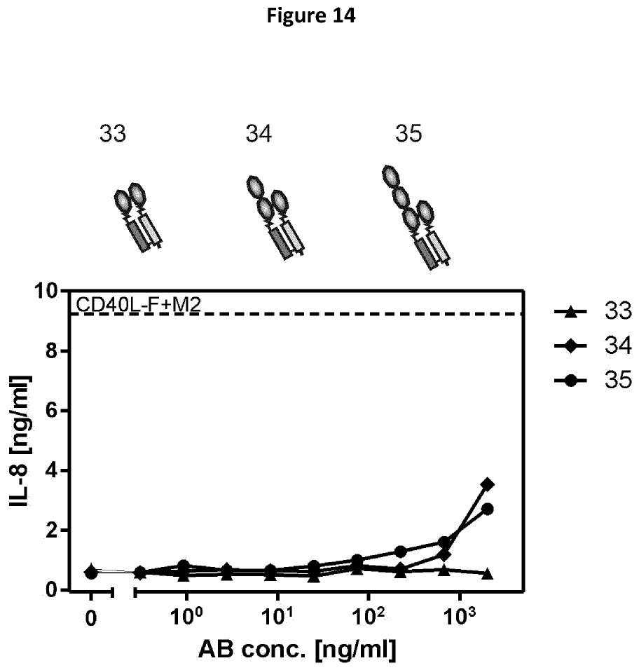

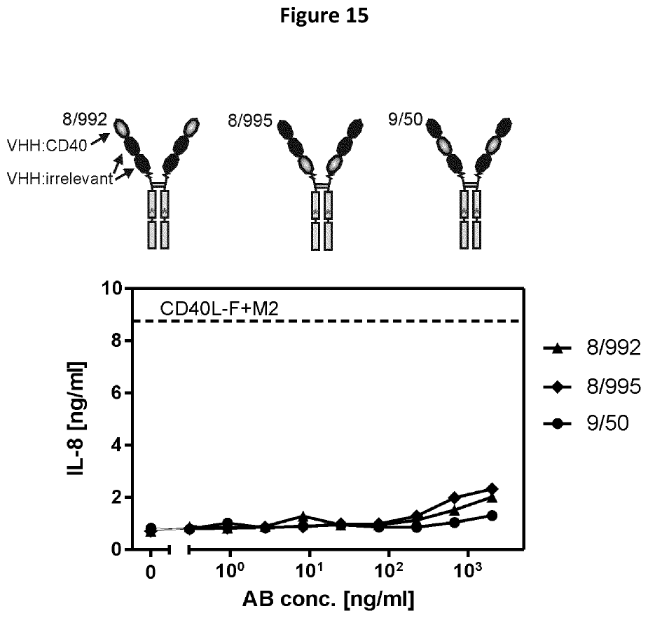

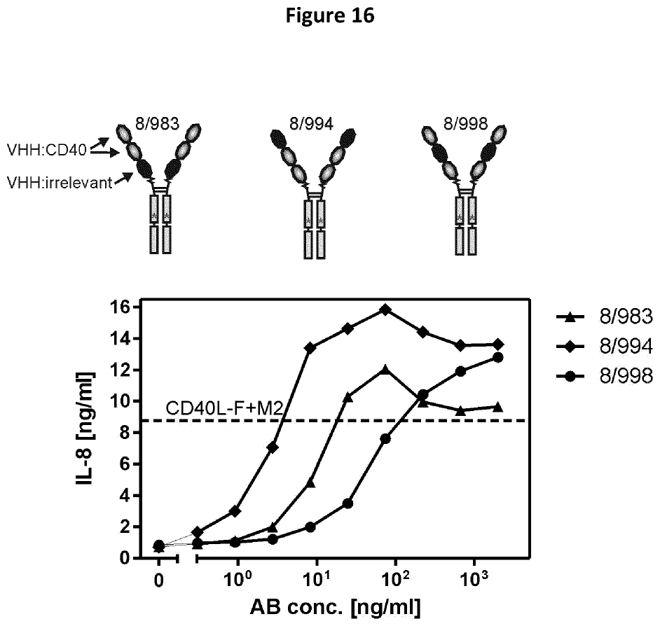

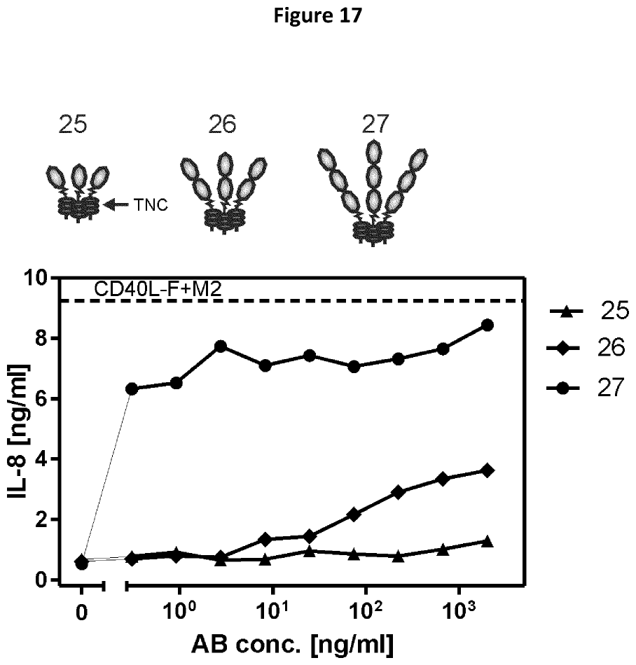

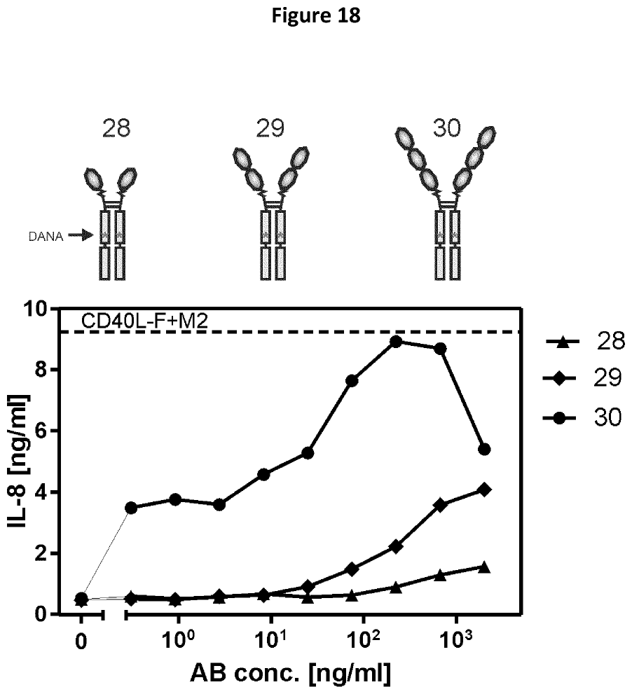

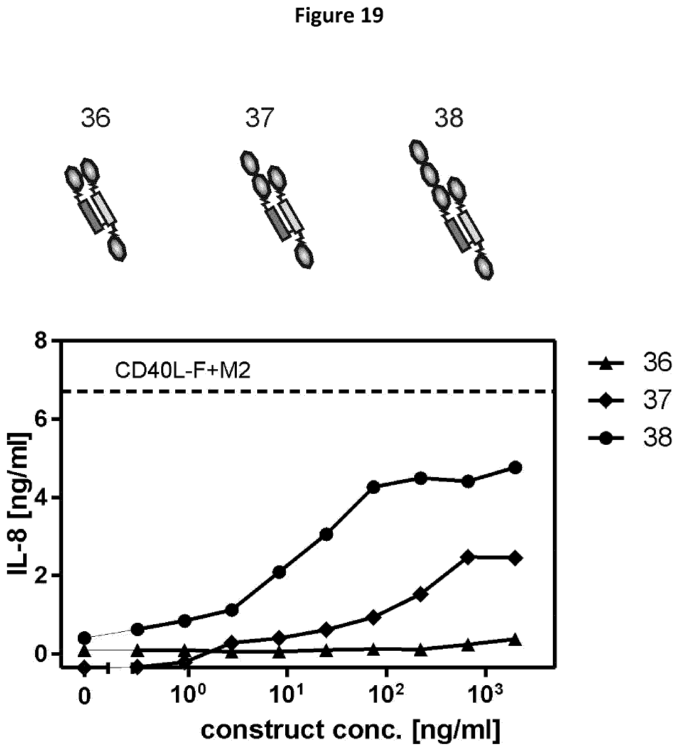

With exception of the mediocre active hexavalent 2×sdAb-TNC fusion protein (construct 26, FIG. 17), all constructs with 6 or more sdAb domains (constructs 17,18 in FIG. 11; constructs 19-21 in FIG. 12; constructs 22 and 23 in FIG. 13, construct 30 FIG. 18) were highly active at low concentrations reaching the activity of oligomerized recombinant soluble CD40L which served as a benchmark. All bi- or trivalent sdAb variants were largely inactive (constructs 33, 34, FIG. 14; constructs 8/992, 8/995, 9/50 FIG. 15; construct 25 FIG. 17; construct 28, FIG. 18). It is worth mentioning that the agonism of tetravalent constructs varied considerably from poorly active (construct 35 FIG. 14) over mediocre active (construct 16, FIG. 11) to largely as active as hexavalent constructs (constructs 8/983, 8/994, 8/998, FIG. 16). The huge variability in the activity of the tetravalent constructs might result from different abilities of the four sdAb domains within a certain construct to bind in parallel to CD40 or by different capacities to bind to CD40 molecules expressed by two neighboring cells (This may also account to the hexavalent version 2×VHH-TNC). Indeed, there is evidence that the cell to cell contact situation significantly increases the ability of soluble ligand molecules and antibodies to engage receptors of the TNFRSF including CD40 (Wajant 2015). To prove the latter, we equipped the bi-, tri- and tetravalent sdAb-Fab1 versions (construct 33-35, FIG. 14) with one additional sdAb domain at the C-terminus (constructs 36-38, FIG. 19). Indeed, this resulted in a significant enhanced activity of the inactive constructs 34 and 35 from FIG. 14.

Conclusion A) 6 Parallel oriented CD40 biding sites ensure strong CD40 agonism. Further increase of binding sites has comparable less impact. Constructs of 4 and presumably 5 binding sits define a transitional area where minor structural differences might have an strong impact on agonism.

Conclusion B) bidirectional oriented CD40 binding sites can compensate to some extend for CD40 binding site number.

### INDUSTRIAL APPLICABILITY

The pharmaceutical compositions, polypeptides, nucleic acids, cells, and products for use in the invention are industrially applicable. For example, they can be used in the manufacture of, or as, pharmaceutical products.

### REFERENCES

Vonderheide R H. CD40 Agonist Antibodies in Cancer Immunotherapy. Annu Rev Med. 2020 Jan. 27; 71:47-58
Li D K, Wang W. Characteristics and clinical trial results of agonistic anti-CD40 antibodies in the treatment of malignancies. Oncol Lett. 2020 Nov. 20(5):176
Bullock T N J. CD40 stimulation as a molecular adjuvant for cancer vaccines and other immunotherapies. Cell Mol Immunol. 2022 Jan. 19(1):14-22.
Wajant H. Principles of antibody-mediated TNF receptor activation. Cell Death Differ. 2015 Nov. 22(11):1727-41.
Mangsbo S M, Broos S, Fletcher E, Veitonmäki N, Furebring C, Dahlén E, Norlen P, Lindstedt M, Tötterman T H, Ellmark P. The human agonistic CD40 antibody ADC-1013 eradicates bladder tumors and generates T-cell-dependent tumor immunity. Clin Cancer Res. 2015 Mar. 1; 21(5):1115-26.
Filbert E L, Björck P K, Srivastava M K, Bahjat F R, Yang X. APX005M, a CD40 agonist antibody with unique epitope specificity and Fc receptor binding profile for optimal therapeutic application. Cancer Immunol Immunother. 2021 July; 70(7):1853-1865.
Grilley-Olson J E, Curti B D, Smith D C et al (2018) SEA-CD40, a non-fucosylated CD40 agonist: Interim results from a phase 1 study in advanced solid tumors. J Clin Oncol 36:3093. https://doi.org/10.1200/JCO.2018.36.15_suppl.3093
Medler J, Nelke J, Weisenberger D, Steinfatt T, Rothaug M, Berr S, Hünig T, Beilhack A, Wajant H. TNFRSF receptor-specific antibody fusion proteins with targeting controlled FcγR-independent agonistic activity. Cell Death Dis. 2019 Mar. 4; 10(3):224.
Chowdhury F, Johnson P W, Glennie M J, Williams A P. Ex vivo assays of dendritic cell activation and cytokine profiles as predictors of in vivo effects in an anti-human CD40 monoclonal antibody ChiLob 7/4 phase I trial. Cancer Immunol Res. 2014 Mar. 2(3):229-40.
Richman L P, Vonderheide R H. Anti-human CD40 monoclonal antibody therapy is potent without FcR crosslinking. Oncoimmunology. 2014 Apr. 29; 3:e28610.
Vitale L A, Thomas L J, He L Z, O'Neill T, Widger J, Crocker A, Sundarapandiyan K, Storey J R, Forsberg E M, Weidlick J, Baronas A R, Gergel L E, Boyer J M, Sisson C, Goldstein J, Marsh H C Jr, Keler T. Development of CDX-1140, an agonist CD40 antibody for cancer immunotherapy. Cancer Immunol Immunother. 2019 February; 68(2):233-245.
White A L, Chan H T, French R R, Willoughby J, Mockridge Cl, Roghanian A, Penfold C A, Booth S G, Dodhy A, Polak M E, Potter E A, Ardern-Jones M R, Verbeek J S, Johnson P W, A I-Shamkhani A, Cragg M S, Beers S A, Glennie M J. Conformation of the human immunoglobulin G2 hinge imparts superagonistic properties to immunostimulatory anticancer antibodies. Cancer Cell. 2015 Jan. 12; 27(1):138-48.
Yu X, Chan HTC, Fisher H, Penfold C A, Kim J, Inzhelevskaya T, Mockridge Cl, French R R, Duriez P J, Douglas L R, English V, Verbeek J S, White A L, Tews I, Glennie M J, Cragg M S. Isotype Switching Converts Anti-CD40 Antagonism to Agonism to Elicit Potent Antitumor Activity. Cancer Cell. 2020 Jun. 8; 37(6):850-866.e7.
Dahan R, Barnhart B C, Li F, Yamniuk A P, Korman A J, Ravetch J V. Therapeutic Activity of Agonistic, Human Anti-CD40 Monoclonal Antibodies Requires Selective FcγR Engagement. Cancer Cell. 2016 Jun. 13; 29(6):820-831.
Yu X, Chan HTC, Orr C M, Dadas O, Booth S G, Dahal L N, Penfold C A, O'Brien L, Mockridge Cl, French R R, Duriez P, Douglas L R, Pearson A R, Cragg M S, Tews I, Glennie M J, White A L. Complex Interplay between Epitope Specificity and Isotype Dictates the Biological Activity of Anti-human CD40 Antibodies. Cancer Cell. 2018 Apr. 9; 33(4):664-675.e4.
Li F, Ravetch J V. Inhibitory Fcγ receptor engagement drives adjuvant and anti-tumor activities of agonistic CD40 antibodies. Science. 2011 Aug. 19; 333(6045):1030-4.
Wilson N S, Yang B, Yang A, Loeser S, Marsters S, Lawrence D, Li Y, Pitti R, Totpal K, Yee S, Ross S, Vernes J M, Lu Y, Adams C, Offringa R, Kelley B, Hymowitz S, Daniel D, Meng G, Ashkenazi A. An Fcγ receptor-dependent mechanism drives antibody-mediated target-receptor signaling in cancer cells. Cancer Cell. 2011 Jan. 18; 19(1):101-13.
Medler J, Kucka K, Melo V, Zhang T, von Rotenhan S, Ulrich J, Bremer E, Hudecek M, Beilhack A, Wajant H. CD40- and 41BB-specific antibody fusion proteins with PDL1 blockade-restricted agonism. Theranostics. 2022 Jan. 1; 12(4):1486-1499.
Nelke J, Medler J, Weisenberger D, Beilhack A, Wajant H. CD40- and CD95-specific antibody single chain-Baff fusion proteins display BaffR-, TACl- and BCMA-restricted agonism. MAbs. 2020 Jan.-Dec. 12(1):1807721.
Ye S, Cohen D, Belmar N A, Choi D, Tan S S, Sho M, Akamatsu Y, Kim H, Iyer R, Cabel J, Lake M, Song D, Harlan J, Zhang C, Fang Y, Wahl A F, Culp P, Hollenbaugh D, Chao D T. A Bispecific Molecule Targeting CD40 and Tumor Antigen Mesothelin Enhances Tumor-Specific Immunity. Cancer Immunol Res. 2019 Nov. 7(11):1864-1875.
Sum E, Rapp M, Fröbel P, Le Clech M, Dürr H, Giusti A M, Perro M, Speziale D, Kunz L, Menietti E, Brünker P, Hopfer U, Lechmann M, Sobieniecki A, Appelt B, Adelfio R, Nicolini V, Freimoser-Grundschober A, Jordaan W, Labiano S, Weber F, Emrich T, Christen F, Essig B, Romero P, Trumpfheller C, Umaña P. Fibroblast Activation Protein α-Targeted CD40 Agonism Abrogates Systemic Toxicity and Enables Administration of High Doses to Induce Effective Antitumor Immunity. Clin Cancer Res. 2021 Jul. 15; 27(14):4036-4053.
Sum E, Rapp M, Dürr H, Mazumdar A, Romero P J, Trumpfheller C, Umaña P. The tumor-targeted CD40 agonist CEA-CD40 promotes T cell priming via a dual mode of action by increasing antigen delivery to dendritic cells and enhancing their activation. J Immunother Cancer. 2022 Mar. 10(3):e003264.
Salomon R, Rotem H, Katzenelenbogen Y, Weiner A, Cohen Saban N, Feferman T, Amit I, Dahan R. Bispecific antibodies increase the therapeutic window of CD40 agonists through selective dendritic cell targeting. Nat Cancer. 2022 Mar. 3(3):287-302.
Clark E A, Yip T C, Ledbetter J A, Yukawa H, Kikutani H, Kishimoto T, Ng M H. CDw40 and BLCa-specific monoclonal antibodies detect two distinct molecules which transmit progression signals to human B lymphocytes. Eur J Immunol. 1988 Mar. 18(3):451-7.
Vonderheide R H, Flaherty K T, Khalil M, Stumacher M S, Bajor D L, Hutnick N A, Sullivan P, Mahany J J, Gallagher M, Kramer A, Green S J, O'Dwyer P J, Running K L, Huhn R D, Antonia S J. Clinical activity and immune modulation in cancer patients treated with CP-870,893, a novel CD40 agonist monoclonal antibody. J Clin Oncol. 2007 Mar. 1; 25(7):876-83.
Vonderheide R H, Glennie M J. Agonistic CD40 antibodies and cancer therapy. Clin Cancer Res. 2013 Mar. 1; 19(5):1035-43.
Vonderheide R H. CD40 Agonist Antibodies in Cancer Immunotherapy. Annu Rev Med. 2020 Jan. 27; 71:47-58.
Wang X, Mathieu M, Brezski R J. IgG Fc engineering to modulate antibody effector functions. Protein Cell. 2018 Jan. 9(1):63-73.
Hayden, M. S.; Ghosh, S. N F-K B, the first quarter-century: remarkable progress and outstanding questions. Genes Dev 2012, 26, 203-234, doi:10.1101/gad.183434.111.
Diebolder C A, Beurskens F J, de Jong R N, Koning R I, Strumane K, Lindorfer M A, Voorhorst M, Ugurlar D, Rosati S, Heck A J, van de Winkel J G, Wilson I A, Koster A J, Taylor R P, Saphire E O, Burton D R, Schuurman J, Gros P, Parren P W. Complement is activated by IgG hexamers assembled at the cell surface. Science. 2014 Mar. 14; 343(6176):1260-3.
de Weerdt I, Lameris R, Scheffer G L, Vree J, de Boer R, Stam A G, van de Ven R, Levin M D, Pals S T, Roovers R C, Parren PWHI, de Gruijl T D, Kater A P, van der Vliet H J. A Bispecific Antibody Antagonizes Prosurvival CD40 Signaling and Promotes Vγ9Vδ2 T cell-Mediated Antitumor Responses in Human B-cell Malignancies. Cancer Immunol Res. 2021 Jan. 9(1):50-61.
Toxicity of an Fc-engineered anti-CD40 antibody is abrogated by intratumoral injection and results in durable antitumor immunity.
Knorr D A, Dahan R, Ravetch J V. Proc Natl Acad Sci USA. 2018 Oct. 23; 115(43):11048-11053.
Local convection-enhanced delivery of an anti-CD40 agonistic monoclonal antibody induces antitumor effects in mouse glioma models.
Shoji T, Saito R, Chonan M, Shibahara I, Sato A, Kanamori M, Sonoda Y, Kondo T, Ishii N, Tominaga T. Neuro Oncol. 2016 August; 18(8):1120-8.
Trimer stabilization, oligomerization, and antibody-mediated cell surface immobilization improve the activity of soluble trimers of CD27L, CD40L, 41BBL, and glucocorticoid-induced TNF receptor ligand.
Wyzgol A, Müller N, Fick A, Munkel S, Grigoleit G U, Pfizenmaier K, Wajant H. J Immunol. 2009 Aug. 1; 183(3):1851-61.
Quantitative analysis of cell surface antigen-antibody interaction using Gaussia princeps luciferase antibody fusion proteins.
Kums J, Nelke J, Ruth B, Schafer V, Siegmund D, Wajant H. MAbs. 2017 April; 9(3):506-520.
Takada Y K, Yu J, Shimoda M, Takada Y. Integrin Binding to the Trimeric Interface of CD40L Plays a Critical Role in CD40/CD40L Signaling. J Immunol. 2019 Sep. 1; 203(5):1383-1391.
Aloui C, Prigent A, Sut C, Tariket S, Hamzeh-Cognasse H, Pozzetto B, Richard Y, Cognasse F, Laradi S, Garraud O. The signaling role of CD40 ligand in platelet biology and in platelet component transfusion. Int J Mol Sci. 2014 Dec. 3; 15(12):22342-64.

1. A multivalent anti-CD40 antibody construct comprising at least 4 antigen binding sites for CD40, wherein the antibody construct is not a natural IgA or IgM molecule.

2. The construct of claim 1, wherein the construct comprises at least one IgG molecule containing two of said antigen binding sites for CD40.

3. The construct of claim 1, wherein the construct comprises a modified IgG molecule, which has been modified by replacing each of the two variable domains of the heavy chain by at least one single-domain antibody (sdAb) containing one of said antigen binding sites for CD40, and by replacing each of the two variable domains of the light chain by at least one single-domain antibody (sdAb) containing one of said antigen binding sites for CD40.

4. The construct of claim 2 or 3, wherein the construct further comprises an scFv or single-domain antibody (sdAb) covalently linked to the C-terminus of one of the two heavy chains of the IgG molecule or modified IgG molecule, the scFv or single-domain antibody (sdAb) containing one of said antigen binding sites for CD40.

5. The construct of claim 4, wherein the construct further comprises an scFv or single-domain antibody (sdAb) covalently linked to the C-terminus of the other of the two heavy chains of the IgG molecule or modified IgG molecule, the scFv or single-domain antibody (sdAb) containing one of said antigen binding sites for CD40.

6. The construct of claim 1, wherein the construct comprises an Fc fragment of an IgG molecule, and wherein each of the two N-termini and/or each of the two C-termini of said Fc fragment is covalently linked to a polypeptide chain comprising 1, 2 or 3, preferably 3 single-domain antibodies (sdAbs), wherein each of the 1, 2 or 3, preferably 3 single-domain antibodies (sdAbs) comprises one of said antigen binding sites for CD40.

7. The construct of any one of the preceding claims, wherein the construct comprises 6 antigen binding sites for CD40.

8. The construct of any one of the preceding claims, wherein the construct comprises 12 antigen binding sites for CD40.

9. The construct according to any one of the preceding claims, wherein any two of the antigen binding sites for CD40 are in parallel orientation.

10. The construct of any one of claims 2 to 9, wherein the construct comprises a trimer of three of the IgG molecules or modified IgG molecules, formed by a trimerization domain which is fused to the C-terminus of each heavy chain of each of the IgG molecules or modified IgG molecules.

11. The construct according to any one of claims 4 to 10, wherein the trimerization domain is the trimerization domain of tenascin-C.

12. The construct according to claim 10, wherein the trimerization domain comprises the amino acid sequence of SEQ ID NO: 60 or a sequence at least 70% identical thereto, preferably at least 80% identical thereto, more preferably at least 85% identical thereto, more preferably at least 90% identical thereto, more preferably at least 93% identical thereto, and more preferably at least 96% identical thereto.

13. The construct according to any one of claims 2-12, wherein the IgG molecule or modified IgG molecule contains an oligomerization mutation.

14. The construct according to claim 13, wherein the oligomerization mutation is an E345R/E430G/S440Y mutation.

15. The construct according to any one of the preceding claims, wherein the construct comprises one or more Fab1 domains, scFv domains, and/or sdAb domains, each containing one antigen binding site for CD40.

16. The construct of any one of claims 2-15, wherein the IgG molecule or modified IgG molecule is IgG1, IgG2, IgG3 or IgG4.

17. The construct of any one of claims 2-16, wherein the IgG molecule or modified IgG molecule is IgG2 or IgG4.

18. The construct according to any one of claims 2-17, wherein the Fc domain of the IgG molecule or modified IgG molecule or the Fc fragment of the IgG molecule contains a mutation reducing FcγR binding.

19. The construct according to claim 18, wherein the mutation is an N297A mutation.

20. A multivalent anti-CD40 antibody construct comprising at least 2 antigen binding sites for CD40, wherein the antibody construct is not a natural IgA or IgM molecule, and wherein two of the antigen binding sites for CD40 are in antiparallel orientation.

21. The construct of claim 20, wherein the domains containing the binding sites are linked by a linker sequence, preferably a peptide linker sequence.

22. The construct according to any one of the preceding claims, wherein the construct is a CD40 agonist in an Fc-independent manner.

23. The construct according to any one of the preceding claims, wherein the construct comprises:
a. an amino acid sequence at least 85% identical to the amino acid sequence of SEQ ID NO: 1 and an amino acid sequence at least 85% identical to the amino acid sequence of SEQ ID NO: 2, preferably an amino acid sequence at least 90% identical to the amino acid sequence of SEQ ID NO: 1 and an amino acid sequence at least 90% identical to the amino acid sequence of SEQ ID NO: 2, more preferably an amino acid sequence at least 95% identical to the amino acid sequence of SEQ ID NO: 1 and an amino acid sequence at least 95% identical to the amino acid sequence of SEQ ID NO: 2, more preferably an amino acid sequence at least 98% identical to the amino acid sequence of SEQ ID NO: 1 and an amino acid sequence at least 98% identical to the amino acid sequence of SEQ ID NO: 2, more preferably an amino acid sequence at least 99% identical to the amino acid sequence of SEQ ID NO:1 and an amino acid sequence at least 99% identical to the amino acid sequence of SEQ ID NO: 2, and most preferably the amino acid sequence of SEQ ID NO: 1 and the amino acid sequence of SEQ ID NO: 2;
b. an amino acid sequence at least 85% identical to the amino acid sequence of SEQ ID NO: 1 and an amino acid sequence at least 85% identical to the amino acid sequence of SEQ ID NO: 3, preferably an amino acid sequence at least 90% identical to the amino acid sequence of SEQ ID NO: 1 and an amino acid sequence at least 90% identical to the amino acid sequence of SEQ ID NO: 3, more preferably an amino acid sequence at least 95% identical to the amino acid sequence of SEQ ID NO: 1 and an amino acid sequence at least 95% identical to the amino acid sequence of SEQ ID NO: 3, more preferably an amino acid sequence at least 98% identical to the amino acid sequence of SEQ ID NO: 1 and an amino acid sequence at least 98% identical to the amino acid sequence of SEQ ID NO: 3, more preferably an amino acid sequence at least 99% identical to the amino acid sequence of SEQ ID NO:1 and an amino acid sequence at least 99% identical to the amino acid sequence of SEQ ID NO: 3, and most preferably the amino acid sequence of SEQ ID NO: 1 and the amino acid sequence of SEQ ID NO: 3;
c. an amino acid sequence at least 85% identical to the amino acid sequence of SEQ ID NO: 1 and an amino acid sequence at least 85% identical to the amino acid sequence of SEQ ID NO: 4, preferably an amino acid sequence at least 90% identical to the amino acid sequence of SEQ ID NO: 1 and an amino acid sequence at least 90% identical to the amino acid sequence of SEQ ID NO: 4, more preferably an amino acid sequence at least 95% identical to the amino acid sequence of SEQ ID NO: 1 and an amino acid sequence at least 95% identical to the amino acid sequence of SEQ ID NO: 4, more preferably an amino acid sequence at least 98% identical to the amino acid sequence of SEQ ID NO: 1 and an amino acid sequence at least 98% identical to the amino acid sequence of SEQ ID NO: 4, more preferably an amino acid sequence at least 99% identical to the amino acid sequence of SEQ ID NO:1 and an amino acid sequence at least 99% identical to the amino acid sequence of SEQ ID NO: 4, and most preferably the amino acid sequence of SEQ ID NO: 1 and the amino acid sequence of SEQ ID NO: 4;
d. an amino acid sequence at least 85% identical to the amino acid sequence of SEQ ID NO: 5 and an amino acid sequence at least 85% identical to the amino acid sequence of SEQ ID NO: 6, preferably an amino acid sequence at least 90% identical to the amino acid sequence of SEQ ID NO: 5 and an amino acid sequence at least 90% identical to the amino acid sequence of SEQ ID NO: 6, more preferably an amino acid sequence at least 95% identical to the amino acid sequence of SEQ ID NO: 5 and an amino acid sequence at least 95% identical to the amino acid sequence of SEQ ID NO: 6, more preferably an amino acid sequence at least 98% identical to the amino acid sequence of SEQ ID NO: 5 and an amino acid sequence at least 98% identical to the amino acid sequence of SEQ ID NO: 6, more preferably an amino acid sequence at least 99% identical to the amino acid sequence of SEQ ID NO:5 and an amino acid sequence at least 99% identical to the amino acid sequence of SEQ ID NO: 6, and most preferably the amino acid sequence of SEQ ID NO: 5 and the amino acid sequence of SEQ ID NO: 6;
e. an amino acid sequence at least 85% identical to the amino acid sequence of SEQ ID NO: 5 and an amino acid sequence at least 85% identical to the amino acid sequence of SEQ ID NO: 7, preferably an amino acid sequence at least 90% identical to the amino acid sequence of SEQ ID NO: 5 and an amino acid sequence at least 90% identical to the amino acid sequence of SEQ ID NO: 7, more preferably an amino acid sequence at least 95% identical to the amino acid sequence of SEQ ID NO: 5 and an amino acid sequence at least 95% identical to the amino acid sequence of SEQ ID NO: 7, more preferably an amino acid sequence at least 98% identical to the amino acid sequence of SEQ ID NO: 5 and an amino acid sequence at least 98% identical to the amino acid sequence of SEQ ID NO: 7, more preferably an amino acid sequence at least 99% identical to the amino acid sequence of SEQ ID NO:5 and an amino acid sequence at least 99% identical to the amino acid sequence of SEQ ID NO: 6, and most preferably the amino acid sequence of SEQ ID NO: 5 and the amino acid sequence of SEQ ID NO: 7;
f. an amino acid sequence at least 85% identical to the amino acid sequence of SEQ ID NO: 5 and an amino acid sequence at least 85% identical to the amino acid sequence of SEQ ID NO: 8, preferably an amino acid sequence at least 90% identical to the amino acid sequence of SEQ ID NO: 5 and an amino acid sequence at least 90% identical to the amino acid sequence of SEQ ID NO: 8, more preferably an amino acid sequence at least 95% identical to the amino acid sequence of SEQ ID NO: 5 and an amino acid sequence at least 95% identical to the amino acid sequence of SEQ ID NO: 8, more preferably an amino acid sequence at least 98% identical to the amino acid sequence of SEQ ID NO: 5 and an amino acid sequence at least 98% identical to the amino acid sequence of SEQ ID NO: 8, more preferably an amino acid sequence at least 99% identical to the amino acid sequence of SEQ ID NO:5 and an amino acid sequence at least 99% identical to the amino acid sequence of SEQ ID NO: 8, and most preferably the amino acid sequence of SEQ ID NO: 5 and the amino acid sequence of SEQ ID NO: 8;
g. an amino acid sequence at least 85% identical to the amino acid sequence of SEQ ID NO: 9 and an amino acid sequence at least 85% identical to the amino acid sequence of SEQ ID NO: 10, preferably an amino acid sequence at least 90% identical to the amino acid sequence of SEQ ID NO: 9 and an amino acid sequence at least 90% identical to the amino acid sequence of SEQ ID NO: 10, more preferably an amino acid sequence at least 95% identical to the amino acid sequence of SEQ ID NO: 9 and an amino acid sequence at least 95% identical to the amino acid sequence of SEQ ID NO: 10, more preferably an amino acid sequence at least 98% identical to the amino acid sequence of SEQ ID NO: 9 and an amino acid sequence at least 98% identical to the amino acid sequence of SEQ ID NO: 10, more preferably an amino acid sequence at least 99% identical to the amino acid sequence of SEQ ID NO: 9 and an amino acid sequence at least 99% identical to the amino acid sequence of SEQ ID NO: 10, and most preferably the amino acid sequence of SEQ ID NO: 9 and the amino acid sequence of SEQ ID NO: 10;
h. an amino acid sequence at least 85% identical to the amino acid sequence of SEQ ID NO: 9 and an amino acid sequence at least 85% identical to the amino acid sequence of SEQ ID NO: 11, preferably an amino acid sequence at least 90% identical to the amino acid sequence of SEQ ID NO: 9 and an amino acid sequence at least 90% identical to the amino acid sequence of SEQ ID NO: 11, more preferably an amino acid sequence at least 95% identical to the amino acid sequence of SEQ ID NO: 9 and an amino acid sequence at least 95% identical to the amino acid sequence of SEQ ID NO: 11, more preferably an amino acid sequence at least 98% identical to the amino acid sequence of SEQ ID NO: 9 and an amino acid sequence at least 98% identical to the amino acid sequence of SEQ ID NO: 11, more preferably an amino acid sequence at least 99% identical to the amino acid sequence of SEQ ID NO: 9 and an amino acid sequence at least 99% identical to the amino acid sequence of SEQ ID NO: 11, and most preferably the amino acid sequence of SEQ ID NO: 9 and the amino acid sequence of SEQ ID NO: 11;
i. an amino acid sequence at least 85% identical to the amino acid sequence of SEQ ID NO: 9 and an amino acid sequence at least 85% identical to the amino acid sequence of SEQ ID NO: 12, preferably an amino acid sequence at least 90% identical to the amino acid sequence of SEQ ID NO: 9 and an amino acid sequence at least 90% identical to the amino acid sequence of SEQ ID NO: 12, more preferably an amino acid sequence at least 95% identical to the amino acid sequence of SEQ ID NO: 9 and an amino acid sequence at least 95% identical to the amino acid sequence of SEQ ID NO: 12, more preferably an amino acid sequence at least 98% identical to the amino acid sequence of SEQ ID NO: 9 and an amino acid sequence at least 98% identical to the amino acid sequence of SEQ ID NO: 12, more preferably an amino acid sequence at least 99% identical to the amino acid sequence of SEQ ID NO: 9 and an amino acid sequence at least 99% identical to the amino acid sequence of SEQ ID NO: 12, and most preferably the amino acid sequence of SEQ ID NO: 9 and the amino acid sequence of SEQ ID NO: 12;
j. an amino acid sequence at least 85% identical to the amino acid sequence of SEQ ID NO: 13 and an amino acid sequence at least 85% identical to the amino acid sequence of SEQ ID NO: 14, preferably an amino acid sequence at least 90% identical to the amino acid sequence of SEQ ID NO: 13 and an amino acid sequence at least 90% identical to the amino acid sequence of SEQ ID NO: 14, more preferably an amino acid sequence at least 95% identical to the amino acid sequence of SEQ ID NO: 13 and an amino acid sequence at least 95% identical to the amino acid sequence of SEQ ID NO: 14, more preferably an amino acid sequence at least 98% identical to the amino acid sequence of SEQ ID NO: 13 and an amino acid sequence at least 98% identical to the amino acid sequence of SEQ ID NO: 14, more preferably an amino acid sequence at least 99% identical to the amino acid sequence of SEQ ID NO: 13 and an amino acid sequence at least 99% identical to the amino acid sequence of SEQ ID NO: 14, and most preferably the amino acid sequence of SEQ ID NO: 13 and the amino acid sequence of SEQ ID NO: 14;
k. an amino acid sequence at least 85% identical to the amino acid sequence of SEQ ID NO: 13 and an amino acid sequence at least 85% identical to the amino acid sequence of SEQ ID NO: 15, preferably an amino acid sequence at least 90% identical to the amino acid sequence of SEQ ID NO: 13 and an amino acid sequence at least 90% identical to the amino acid sequence of SEQ ID NO: 15, more preferably an amino acid sequence at least 95% identical to the amino acid sequence of SEQ ID NO: 13 and an amino acid sequence at least 95% identical to the amino acid sequence of SEQ ID NO: 15, more preferably an amino acid sequence at least 98% identical to the amino acid sequence of SEQ ID NO: 13 and an amino acid sequence at least 98% identical to the amino acid sequence of SEQ ID NO: 15, more preferably an amino acid sequence at least 99% identical to the amino acid sequence of SEQ ID NO: 13 and an amino acid sequence at least 99% identical to the amino acid sequence of SEQ ID NO: 15, and most preferably the amino acid sequence of SEQ ID NO: 13 and the amino acid sequence of SEQ ID NO: 15;
l. an amino acid sequence at least 85% identical to the amino acid sequence of SEQ ID NO: 13 and an amino acid sequence at least 85% identical to the amino acid sequence of SEQ ID NO: 16, preferably an amino acid sequence at least 90% identical to the amino acid sequence of SEQ ID NO: 13 and an amino acid sequence at least 90% identical to the amino acid sequence of SEQ ID NO: 16, more preferably an amino acid sequence at least 95% identical to the amino acid sequence of SEQ ID NO: 13 and an amino acid sequence at least 95% identical to the amino acid sequence of SEQ ID NO: 16, more preferably an amino acid sequence at least 98% identical to the amino acid sequence of SEQ ID NO: 13 and an amino acid sequence at least 98% identical to the amino acid sequence of SEQ ID NO: 16, more preferably an amino acid sequence at least 99% identical to the amino acid sequence of SEQ ID NO: 13 and an amino acid sequence at least 99% identical to the amino acid sequence of SEQ ID NO: 16, and most preferably the amino acid sequence of SEQ ID NO: 13 and the amino acid sequence of SEQ ID NO: 16;
m. an amino acid sequence at least 85% identical to the amino acid sequence of SEQ ID NO: 17 and an amino acid sequence at least 85% identical to the amino acid sequence of SEQ ID NO: 18, preferably an amino acid sequence at least 90% identical to the amino acid sequence of SEQ ID NO: 17 and an amino acid sequence at least 90% identical to the amino acid sequence of SEQ ID NO: 18, more preferably an amino acid sequence at least 95% identical to the amino acid sequence of SEQ ID NO: 17 and an amino acid sequence at least 95% identical to the amino acid sequence of SEQ ID NO: 18, more preferably an amino acid sequence at least 98% identical to the amino acid sequence of SEQ ID NO: 17 and an amino acid sequence at least 98% identical to the amino acid sequence of SEQ ID NO: 18, more preferably an amino acid sequence at least 99% identical to the amino acid sequence of SEQ ID NO: 17 and an amino acid sequence at least 99% identical to the amino acid sequence of SEQ ID NO: 18, and most preferably the amino acid sequence of SEQ ID NO: 17 and the amino acid sequence of SEQ ID NO: 18;
n. an amino acid sequence at least 85% identical to the amino acid sequence of SEQ ID NO: 17 and an amino acid sequence at least 85% identical to the amino acid sequence of SEQ ID NO: 19, preferably an amino acid sequence at least 90% identical to the amino acid sequence of SEQ ID NO: 17 and an amino acid sequence at least 90% identical to the amino acid sequence of SEQ ID NO: 19, more preferably an amino acid sequence at least 95% identical to the amino acid sequence of SEQ ID NO: 17 and an amino acid sequence at least 95% identical to the amino acid sequence of SEQ ID NO: 19, more preferably an amino acid sequence at least 98% identical to the amino acid sequence of SEQ ID NO: 17 and an amino acid sequence at least 98% identical to the amino acid sequence of SEQ ID NO: 19, more preferably an amino acid sequence at least 99% identical to the amino acid sequence of SEQ ID NO: 17 and an amino acid sequence at least 99% identical to the amino acid sequence of SEQ ID NO: 19, and most preferably the amino acid sequence of SEQ ID NO: 17 and the amino acid sequence of SEQ ID NO: 19;
o. an amino acid sequence at least 85% identical to the amino acid sequence of SEQ ID NO: 17 and an amino acid sequence at least 85% identical to the amino acid sequence of SEQ ID NO: 20, preferably an amino acid sequence at least 90% identical to the amino acid sequence of SEQ ID NO: 17 and an amino acid sequence at least 90% identical to the amino acid sequence of SEQ ID NO: 20, more preferably an amino acid sequence at least 95% identical to the amino acid sequence of SEQ ID NO: 17 and an amino acid sequence at least 95% identical to the amino acid sequence of SEQ ID NO: 20, more preferably an amino acid sequence at least 98% identical to the amino acid sequence of SEQ ID NO: 17 and an amino acid sequence at least 98% identical to the amino acid sequence of SEQ ID NO: 20, more preferably an amino acid sequence at least 99% identical to the amino acid sequence of SEQ ID NO: 17 and an amino acid sequence at least 99% identical to the amino acid sequence of SEQ ID NO: 20, and most preferably the amino acid sequence of SEQ ID NO: 17 and the amino acid sequence of SEQ ID NO: 20;
p. an amino acid sequence at least 85% identical to the amino acid sequence of SEQ ID NO: 21 and an amino acid sequence at least 85% identical to the amino acid sequence of SEQ ID NO: 24, preferably an amino acid sequence at least 90% identical to the amino acid sequence of SEQ ID NO: 21 and an amino acid sequence at least 90% identical to the amino acid sequence of SEQ ID NO: 24, more preferably an amino acid sequence at least 95% identical to the amino acid sequence of SEQ ID NO: 21 and an amino acid sequence at least 95% identical to the amino acid sequence of SEQ ID NO: 24, more preferably an amino acid sequence at least 98% identical to the amino acid sequence of SEQ ID NO: 21 and an amino acid sequence at least 98% identical to the amino acid sequence of SEQ ID NO: 24, more preferably an amino acid sequence at least 99% identical to the amino acid sequence of SEQ ID NO: 21 and an amino acid sequence at least 99% identical to the amino acid sequence of SEQ ID NO: 24, and most preferably the amino acid sequence of SEQ ID NO: 21 and the amino acid sequence of SEQ ID NO: 24;
q. an amino acid sequence at least 85% identical to the amino acid sequence of SEQ ID NO: 21 and an amino acid sequence at least 85% identical to the amino acid sequence of SEQ ID NO: 25, preferably an amino acid sequence at least 90% identical to the amino acid sequence of SEQ ID NO: 21 and an amino acid sequence at least 90% identical to the amino acid sequence of SEQ ID NO: 25, more preferably an amino acid sequence at least 95% identical to the amino acid sequence of SEQ ID NO: 21 and an amino acid sequence at least 95% identical to the amino acid sequence of SEQ ID NO: 25, more preferably an amino acid sequence at least 98% identical to the amino acid sequence of SEQ ID NO: 21 and an amino acid sequence at least 98% identical to the amino acid sequence of SEQ ID NO: 25, more preferably an amino acid sequence at least 99% identical to the amino acid sequence of SEQ ID NO: 21 and an amino acid sequence at least 99% identical to the amino acid sequence of SEQ ID NO: 25, and most preferably the amino acid sequence of SEQ ID NO: 21 and the amino acid sequence of SEQ ID NO: 25;
r. an amino acid sequence at least 85% identical to the amino acid sequence of SEQ ID NO: 21 and an amino acid sequence at least 85% identical to the amino acid sequence of SEQ ID NO: 26, preferably an amino acid sequence at least 90% identical to the amino acid sequence of SEQ ID NO: 21 and an amino acid sequence at least 90% identical to the amino acid sequence of SEQ ID NO: 26, more preferably an amino acid sequence at least 95% identical to the amino acid sequence of SEQ ID NO: 21 and an amino acid sequence at least 95% identical to the amino acid sequence of SEQ ID NO: 26, more preferably an amino acid sequence at least 98% identical to the amino acid sequence of SEQ ID NO: 21 and an amino acid sequence at least 98% identical to the amino acid sequence of SEQ ID NO: 26, more preferably an amino acid sequence at least 99% identical to the amino acid sequence of SEQ ID NO: 21 and an amino acid sequence at least 99% identical to the amino acid sequence of SEQ ID NO: 26, and most preferably the amino acid sequence of SEQ ID NO: 21 and the amino acid sequence of SEQ ID NO: 26;
s. an amino acid sequence at least 85% identical to the amino acid sequence of SEQ ID NO: 22 and an amino acid sequence at least 85% identical to the amino acid sequence of SEQ ID NO: 24, preferably an amino acid sequence at least 90% identical to the amino acid sequence of SEQ ID NO: 22 and an amino acid sequence at least 90% identical to the amino acid sequence of SEQ ID NO: 24, more preferably an amino acid sequence at least 95% identical to the amino acid sequence of SEQ ID NO: 22 and an amino acid sequence at least 95% identical to the amino acid sequence of SEQ ID NO: 24, more preferably an amino acid sequence at least 98% identical to the amino acid sequence of SEQ ID NO: 22 and an amino acid sequence at least 98% identical to the amino acid sequence of SEQ ID NO: 24, more preferably an amino acid sequence at least 99% identical to the amino acid sequence of SEQ ID NO: 22 and an amino acid sequence at least 99% identical to the amino acid sequence of SEQ ID NO: 24, and most preferably the amino acid sequence of SEQ ID NO: 22 and the amino acid sequence of SEQ ID NO: 24;
t. an amino acid sequence at least 85% identical to the amino acid sequence of SEQ ID NO: 22 and an amino acid sequence at least 85% identical to the amino acid sequence of SEQ ID NO: 25, preferably an amino acid sequence at least 90% identical to the amino acid sequence of SEQ ID NO: 22 and an amino acid sequence at least 90% identical to the amino acid sequence of SEQ ID NO: 25, more preferably an amino acid sequence at least 95% identical to the amino acid sequence of SEQ ID NO: 22 and an amino acid sequence at least 95% identical to the amino acid sequence of SEQ ID NO: 25, more preferably an amino acid sequence at least 98% identical to the amino acid sequence of SEQ ID NO: 22 and an amino acid sequence at least 98% identical to the amino acid sequence of SEQ ID NO: 25, more preferably an amino acid sequence at least 99% identical to the amino acid sequence of SEQ ID NO: 22 and an amino acid sequence at least 99% identical to the amino acid sequence of SEQ ID NO: 25, and most preferably the amino acid sequence of SEQ ID NO: 22 and the amino acid sequence of SEQ ID NO: 25;
u. an amino acid sequence at least 85% identical to the amino acid sequence of SEQ ID NO: 22 and an amino acid sequence at least 85% identical to the amino acid sequence of SEQ ID NO: 26, preferably an amino acid sequence at least 90% identical to the amino acid sequence of SEQ ID NO: 22 and an amino acid sequence at least 90% identical to the amino acid sequence of SEQ ID NO: 26, more preferably an amino acid sequence at least 95% identical to the amino acid sequence of SEQ ID NO: 22 and an amino acid sequence at least 95% identical to the amino acid sequence of SEQ ID NO: 26, more preferably an amino acid sequence at least 98% identical to the amino acid sequence of SEQ ID NO: 22 and an amino acid sequence at least 98% identical to the amino acid sequence of SEQ ID NO: 26, more preferably an amino acid sequence at least 99% identical to the amino acid sequence of SEQ ID NO: 22 and an amino acid sequence at least 99% identical to the amino acid sequence of SEQ ID NO: 26, and most preferably the amino acid sequence of SEQ ID NO: 22 and the amino acid sequence of SEQ ID NO: 26;
v. an amino acid sequence at least 85% identical to the amino acid sequence of SEQ ID NO: 23 and an amino acid sequence at least 85% identical to the amino acid sequence of SEQ ID NO: 24, preferably an amino acid sequence at least 90% identical to the amino acid sequence of SEQ ID NO: 23 and an amino acid sequence at least 90% identical to the amino acid sequence of SEQ ID NO: 24, more preferably an amino acid sequence at least 95% identical to the amino acid sequence of SEQ ID NO: 23 and an amino acid sequence at least 95% identical to the amino acid sequence of SEQ ID NO: 24, more preferably an amino acid sequence at least 98% identical to the amino acid sequence of SEQ ID NO: 23 and an amino acid sequence at least 98% identical to the amino acid sequence of SEQ ID NO: 24, more preferably an amino acid sequence at least 99% identical to the amino acid sequence of SEQ ID NO: 23 and an amino acid sequence at least 99% identical to the amino acid sequence of SEQ ID NO: 24, and most preferably the amino acid sequence of SEQ ID NO: 23 and the amino acid sequence of SEQ ID NO: 24;
w. an amino acid sequence at least 85% identical to the amino acid sequence of SEQ ID NO: 23 and an amino acid sequence at least 85% identical to the amino acid sequence of SEQ ID NO: 25, preferably an amino acid sequence at least 90% identical to the amino acid sequence of SEQ ID NO: 23 and an amino acid sequence at least 90% identical to the amino acid sequence of SEQ ID NO: 25, more preferably an amino acid sequence at least 95% identical to the amino acid sequence of SEQ ID NO: 23 and an amino acid sequence at least 95% identical to the amino acid sequence of SEQ ID NO: 25, more preferably an amino acid sequence at least 98% identical to the amino acid sequence of SEQ ID NO: 23 and an amino acid sequence at least 98% identical to the amino acid sequence of SEQ ID NO: 25, more preferably an amino acid sequence at least 99% identical to the amino acid sequence of SEQ ID NO: 23 and an amino acid sequence at least 99% identical to the amino acid sequence of SEQ ID NO: 25, and most preferably the amino acid sequence of SEQ ID NO: 23 and the amino acid sequence of SEQ ID NO: 25;
x. an amino acid sequence at least 85% identical to the amino acid sequence of SEQ ID NO: 23 and an amino acid sequence at least 85% identical to the amino acid sequence of SEQ ID NO: 26, preferably an amino acid sequence at least 90% identical to the amino acid sequence of SEQ ID NO: 23 and an amino acid sequence at least 90% identical to the amino acid sequence of SEQ ID NO: 26, more preferably an amino acid sequence at least 95% identical to the amino acid sequence of SEQ ID NO: 23 and an amino acid sequence at least 95% identical to the amino acid sequence of SEQ ID NO: 26, more preferably an amino acid sequence at least 98% identical to the amino acid sequence of SEQ ID NO: 23 and an amino acid sequence at least 98% identical to the amino acid sequence of SEQ ID NO: 26, more preferably an amino acid sequence at least 99% identical to the amino acid sequence of SEQ ID NO: 23 and an amino acid sequence at least 99% identical to the amino acid sequence of SEQ ID NO: 26, and most preferably the amino acid sequence of SEQ ID NO: 23 and the amino acid sequence of SEQ ID NO: 26;
y. an amino acid sequence at least 85% identical to the amino acid sequence of SEQ ID NO: 27, preferably an amino acid sequence at least 90% identical to the amino acid sequence of SEQ ID NO: 27, more preferably an amino acid sequence at least 95% identical to the amino acid sequence of SEQ ID NO: 27, more preferably an amino acid sequence at least 98% identical to the amino acid sequence of SEQ ID NO: 27, more preferably an amino acid sequence at least 99% identical to the amino acid sequence of SEQ ID NO: 27, and most preferably the amino acid sequence of SEQ ID NO: 27;
z. an amino acid sequence at least 85% identical to the amino acid sequence of SEQ ID NO: 28, preferably an amino acid sequence at least 90% identical to the amino acid sequence of SEQ ID NO: 28, more preferably an amino acid sequence at least 95% identical to the amino acid sequence of SEQ ID NO: 28, more preferably an amino acid sequence at least 98% identical to the amino acid sequence of SEQ ID NO: 28, more preferably an amino acid sequence at least 99% identical to the amino acid sequence of SEQ ID NO: 28, and most preferably the amino acid sequence of SEQ ID NO: 28;
aa. an amino acid sequence at least 85% identical to the amino acid sequence of SEQ ID NO: 29, preferably an amino acid sequence at least 90% identical to the amino acid sequence of SEQ ID NO: 29, more preferably an amino acid sequence at least 95% identical to the amino acid sequence of SEQ ID NO: 29, more preferably an amino acid sequence at least 98% identical to the amino acid sequence of SEQ ID NO: 29, more preferably an amino acid sequence at least 99% identical to the amino acid sequence of SEQ ID NO: 29, and most preferably the amino acid sequence of SEQ ID NO: 29;
bb. an amino acid sequence at least 85% identical to the amino acid sequence of SEQ ID NO: 31, preferably an amino acid sequence at least 90% identical to the amino acid sequence of SEQ ID NO: 31, more preferably an amino acid sequence at least 95% identical to the amino acid sequence of SEQ ID NO: 31, more preferably an amino acid sequence at least 98% identical to the amino acid sequence of SEQ ID NO: 31, more preferably an amino acid sequence at least 99% identical to the amino acid sequence of SEQ ID NO: 31, and most preferably the amino acid sequence of SEQ ID NO: 31;
cc. an amino acid sequence at least 85% identical to the amino acid sequence of SEQ ID NO: 32, preferably an amino acid sequence at least 90% identical to the amino acid sequence of SEQ ID NO: 32, more preferably an amino acid sequence at least 95% identical to the amino acid sequence of SEQ ID NO: 32, more preferably an amino acid sequence at least 98% identical to the amino acid sequence of SEQ ID NO: 32, more preferably an amino acid sequence at least 99% identical to the amino acid sequence of SEQ ID NO: 32, and most preferably the amino acid sequence of SEQ ID NO: 32;
dd. an amino acid sequence at least 85% identical to the amino acid sequence of SEQ ID NO: 33, preferably an amino acid sequence at least 90% identical to the amino acid sequence of SEQ ID NO: 33, more preferably an amino acid sequence at least 95% identical to the amino acid sequence of SEQ ID NO: 33, more preferably an amino acid sequence at least 98% identical to the amino acid sequence of SEQ ID NO: 33, more preferably an amino acid sequence at least 99% identical to the amino acid sequence of SEQ ID NO: 33, and most preferably the amino acid sequence of SEQ ID NO: 33;
ee. an amino acid sequence at least 85% identical to the amino acid sequence of SEQ ID NO: 34, preferably an amino acid sequence at least 90% identical to the amino acid sequence of SEQ ID NO: 34, more preferably an amino acid sequence at least 95% identical to the amino acid sequence of SEQ ID NO: 34, more preferably an amino acid sequence at least 98% identical to the amino acid sequence of SEQ ID NO: 34, more preferably an amino acid sequence at least 99% identical to the amino acid sequence of SEQ ID NO: 34, and most preferably the amino acid sequence of SEQ ID NO: 34;
ff. an amino acid sequence at least 85% identical to the amino acid sequence of SEQ ID NO: 35, preferably an amino acid sequence at least 90% identical to the amino acid sequence of SEQ ID NO: 35, more preferably an amino acid sequence at least 95% identical to the amino acid sequence of SEQ ID NO: 35, more preferably an amino acid sequence at least 98% identical to the amino acid sequence of SEQ ID NO: 35, more preferably an amino acid sequence at least 99% identical to the amino acid sequence of SEQ ID NO: 35, and most preferably the amino acid sequence of SEQ ID NO: 35;
gg. an amino acid sequence at least 85% identical to the amino acid sequence of SEQ ID NO: 21 and an amino acid sequence at least 85% identical to the amino acid sequence of SEQ ID NO: 36, preferably an amino acid sequence at least 90% identical to the amino acid sequence of SEQ ID NO: 21 and an amino acid sequence at least 90% identical to the amino acid sequence of SEQ ID NO: 36, more preferably an amino acid sequence at least 95% identical to the amino acid sequence of SEQ ID NO: 21 and an amino acid sequence at least 95% identical to the amino acid sequence of SEQ ID NO: 36, more preferably an amino acid sequence at least 98% identical to the amino acid sequence of SEQ ID NO: 21 and an amino acid sequence at least 98% identical to the amino acid sequence of SEQ ID NO: 36, more preferably an amino acid sequence at least 99% identical to the amino acid sequence of SEQ ID NO: 21 and an amino acid sequence at least 99% identical to the amino acid sequence of SEQ ID NO: 36, and most preferably the amino acid sequence of SEQ ID NO: 21 and the amino acid sequence of SEQ ID NO: 36;
hh. an amino acid sequence at least 85% identical to the amino acid sequence of SEQ ID NO: 22 and an amino acid sequence at least 85% identical to the amino acid sequence of SEQ ID NO: 36, preferably an amino acid sequence at least 90% identical to the amino acid sequence of SEQ ID NO: 22 and an amino acid sequence at least 90% identical to the amino acid sequence of SEQ ID NO: 36, more preferably an amino acid sequence at least 95% identical to the amino acid sequence of SEQ ID NO: 22 and an amino acid sequence at least 95% identical to the amino acid sequence of SEQ ID NO: 36, more preferably an amino acid sequence at least 98% identical to the amino acid sequence of SEQ ID NO: 22 and an amino acid sequence at least 98% identical to the amino acid sequence of SEQ ID NO: 36, more preferably an amino acid sequence at least 99% identical to the amino acid sequence of SEQ ID NO: 22 and an amino acid sequence at least 99% identical to the amino acid sequence of SEQ ID NO: 36, and most preferably the amino acid sequence of SEQ ID NO: 22 and the amino acid sequence of SEQ ID NO: 36;
ii. an amino acid sequence at least 85% identical to the amino acid sequence of SEQ ID NO: 23 and an amino acid sequence at least 85% identical to the amino acid sequence of SEQ ID NO: 36, preferably an amino acid sequence at least 90% identical to the amino acid sequence of SEQ ID NO: 23 and an amino acid sequence at least 90% identical to the amino acid sequence of SEQ ID NO: 36, more preferably an amino acid sequence at least 95% identical to the amino acid sequence of SEQ ID NO: 23 and an amino acid sequence at least 95% identical to the amino acid sequence of SEQ ID NO: 36, more preferably an amino acid sequence at least 98% identical to the amino acid sequence of SEQ ID NO: 23 and an amino acid sequence at least 98% identical to the amino acid sequence of SEQ ID NO: 36, more preferably an amino acid sequence at least 99% identical to the amino acid sequence of SEQ ID NO: 23 and an amino acid sequence at least 99% identical to the amino acid sequence of SEQ ID NO: 36, and most preferably the amino acid sequence of SEQ ID NO: 23 and the amino acid sequence of SEQ ID NO: 36;
jj. an amino acid sequence at least 85% identical to the amino acid sequence of SEQ ID NO: 21 and an amino acid sequence at least 85% identical to the amino acid sequence of SEQ ID NO: 39, preferably an amino acid sequence at least 90% identical to the amino acid sequence of SEQ ID NO: 21 and an amino acid sequence at least 90% identical to the amino acid sequence of SEQ ID NO: 39, more preferably an amino acid sequence at least 95% identical to the amino acid sequence of SEQ ID NO: 21 and an amino acid sequence at least 95% identical to the amino acid sequence of SEQ ID NO: 39, more preferably an amino acid sequence at least 98% identical to the amino acid sequence of SEQ ID NO: 21 and an amino acid sequence at least 98% identical to the amino acid sequence of SEQ ID NO: 39, more preferably an amino acid sequence at least 99% identical to the amino acid sequence of SEQ ID NO: 21 and an amino acid sequence at least 99% identical to the amino acid sequence of SEQ ID NO: 39, and most preferably the amino acid sequence of SEQ ID NO: 21 and the amino acid sequence of SEQ ID NO: 39;
kk. an amino acid sequence at least 85% identical to the amino acid sequence of SEQ ID NO: 22 and an amino acid sequence at least 85% identical to the amino acid sequence of SEQ ID NO: 39, preferably an amino acid sequence at least 90% identical to the amino acid sequence of SEQ ID NO: 22 and an amino acid sequence at least 90% identical to the amino acid sequence of SEQ ID NO: 39, more preferably an amino acid sequence at least 95% identical to the amino acid sequence of SEQ ID NO: 22 and an amino acid sequence at least 95% identical to the amino acid sequence of SEQ ID NO: 39, more preferably an amino acid sequence at least 98% identical to the amino acid sequence of SEQ ID NO: 22 and an amino acid sequence at least 98% identical to the amino acid sequence of SEQ ID NO: 39, more preferably an amino acid sequence at least 99% identical to the amino acid sequence of SEQ ID NO: 22 and an amino acid sequence at least 99% identical to the amino acid sequence of SEQ ID NO: 39, and most preferably the amino acid sequence of SEQ ID NO: 22 and the amino acid sequence of SEQ ID NO: 39;
ll. an amino acid sequence at least 85% identical to the amino acid sequence of SEQ ID NO: 23 and an amino acid sequence at least 85% identical to the amino acid sequence of SEQ ID NO: 39, preferably an amino acid sequence at least 90% identical to the amino acid sequence of SEQ ID NO: 23 and an amino acid sequence at least 90% identical to the amino acid sequence of SEQ ID NO: 39, more preferably an amino acid sequence at least 95% identical to the amino acid sequence of SEQ ID NO: 23 and an amino acid sequence at least 95% identical to the amino acid sequence of SEQ ID NO: 39, more preferably an amino acid sequence at least 98% identical to the amino acid sequence of SEQ ID NO: 23 and an amino acid sequence at least 98% identical to the amino acid sequence of SEQ ID NO: 39, more preferably an amino acid sequence at least 99% identical to the amino acid sequence of SEQ ID NO: 23 and an amino acid sequence at least 99% identical to the amino acid sequence of SEQ ID NO: 39, and most preferably the amino acid sequence of SEQ ID NO: 23 and the amino acid sequence of SEQ ID NO: 39;
mm. an amino acid sequence at least 85% identical to the amino acid sequence of SEQ ID NO: 21 and an amino acid sequence at least 85% identical to the amino acid sequence of SEQ ID NO: 40, preferably an amino acid sequence at least 90% identical to the amino acid sequence of SEQ ID NO: 21 and an amino acid sequence at least 90% identical to the amino acid sequence of SEQ ID NO: 40, more preferably an amino acid sequence at least 95% identical to the amino acid sequence of SEQ ID NO: 21 and an amino acid sequence at least 95% identical to the amino acid sequence of SEQ ID NO: 40, more preferably an amino acid sequence at least 98% identical to the amino acid sequence of SEQ ID NO: 21 and an amino acid sequence at least 98% identical to the amino acid sequence of SEQ ID NO: 40, more preferably an amino acid sequence at least 99% identical to the amino acid sequence of SEQ ID NO: 21 and an amino acid sequence at least 99% identical to the amino acid sequence of SEQ ID NO: 40, and most preferably the amino acid sequence of SEQ ID NO: 21 and the amino acid sequence of SEQ ID NO: 40;
nn. an amino acid sequence at least 85% identical to the amino acid sequence of SEQ ID NO: 22 and an amino acid sequence at least 85% identical to the amino acid sequence of SEQ ID NO: 40, preferably an amino acid sequence at least 90% identical to the amino acid sequence of SEQ ID NO: 22 and an amino acid sequence at least 90% identical to the amino acid sequence of SEQ ID NO: 40, more preferably an amino acid sequence at least 95% identical to the amino acid sequence of SEQ ID NO: 22 and an amino acid sequence at least 95% identical to the amino acid sequence of SEQ ID NO: 40, more preferably an amino acid sequence at least 98% identical to the amino acid sequence of SEQ ID NO: 22 and an amino acid sequence at least 98% identical to the amino acid sequence of SEQ ID NO: 40, more preferably an amino acid sequence at least 99% identical to the amino acid sequence of SEQ ID NO: 22 and an amino acid sequence at least 99% identical to the amino acid sequence of SEQ ID NO: 40, and most preferably the amino acid sequence of SEQ ID NO: 22 and the amino acid sequence of SEQ ID NO: 40;
oo. an amino acid sequence at least 85% identical to the amino acid sequence of SEQ ID NO: 23 and an amino acid sequence at least 85% identical to the amino acid sequence of SEQ ID NO: 40, preferably an amino acid sequence at least 90% identical to the amino acid sequence of SEQ ID NO: 23 and an amino acid sequence at least 90% identical to the amino acid sequence of SEQ ID NO: 40, more preferably an amino acid sequence at least 95% identical to the amino acid sequence of SEQ ID NO: 23 and an amino acid sequence at least 95% identical to the amino acid sequence of SEQ ID NO: 40, more preferably an amino acid sequence at least 98% identical to the amino acid sequence of SEQ ID NO: 23 and an amino acid sequence at least 98% identical to the amino acid sequence of SEQ ID NO: 40, more preferably an amino acid sequence at least 99% identical to the amino acid sequence of SEQ ID NO: 23 and an amino acid sequence at least 99% identical to the amino acid sequence of SEQ ID NO: 40, and most preferably the amino acid sequence of SEQ ID NO: 23 and the amino acid sequence of SEQ ID NO: 40;
pp. an amino acid sequence at least 85% identical to the amino acid sequence of SEQ ID NO: 21 and an amino acid sequence at least 85% identical to the amino acid sequence of SEQ ID NO: 41, preferably an amino acid sequence at least 90% identical to the amino acid sequence of SEQ ID NO: 21 and an amino acid sequence at least 90% identical to the amino acid sequence of SEQ ID NO: 41, more preferably an amino acid sequence at least 95% identical to the amino acid sequence of SEQ ID NO: 21 and an amino acid sequence at least 95% identical to the amino acid sequence of SEQ ID NO: 41, more preferably an amino acid sequence at least 98% identical to the amino acid sequence of SEQ ID NO: 21 and an amino acid sequence at least 98% identical to the amino acid sequence of SEQ ID NO: 41, more preferably an amino acid sequence at least 99% identical to the amino acid sequence of SEQ ID NO: 21 and an amino acid sequence at least 99% identical to the amino acid sequence of SEQ ID NO: 41, and most preferably the amino acid sequence of SEQ ID NO: 21 and the amino acid sequence of SEQ ID NO: 41;
qq. an amino acid sequence at least 85% identical to the amino acid sequence of SEQ ID NO: 22 and an amino acid sequence at least 85% identical to the amino acid sequence of SEQ ID NO: 41, preferably an amino acid sequence at least 90% identical to the amino acid sequence of SEQ ID NO: 22 and an amino acid sequence at least 90% identical to the amino acid sequence of SEQ ID NO: 41, more preferably an amino acid sequence at least 95% identical to the amino acid sequence of SEQ ID NO: 22 and an amino acid sequence at least 95% identical to the amino acid sequence of SEQ ID NO: 41, more preferably an amino acid sequence at least 98% identical to the amino acid sequence of SEQ ID NO: 22 and an amino acid sequence at least 98% identical to the amino acid sequence of SEQ ID NO: 41, more preferably an amino acid sequence at least 99% identical to the amino acid sequence of SEQ ID NO: 22 and an amino acid sequence at least 99% identical to the amino acid sequence of SEQ ID NO: 41, and most preferably the amino acid sequence of SEQ ID NO: 22 and the amino acid sequence of SEQ ID NO: 41;
rr. an amino acid sequence at least 85% identical to the amino acid sequence of SEQ ID NO: 23 and an amino acid sequence at least 85% identical to the amino acid sequence of SEQ ID NO: 41, preferably an amino acid sequence at least 90% identical to the amino acid sequence of SEQ ID NO: 23 and an amino acid sequence at least 90% identical to the amino acid sequence of SEQ ID NO: 41, more preferably an amino acid sequence at least 95% identical to the amino acid sequence of SEQ ID NO: 23 and an amino acid sequence at least 95% identical to the amino acid sequence of SEQ ID NO: 41, more preferably an amino acid sequence at least 98% identical to the amino acid sequence of SEQ ID NO: 23 and an amino acid sequence at least 98% identical to the amino acid sequence of SEQ ID NO: 41, more preferably an amino acid sequence at least 99% identical to the amino acid sequence of SEQ ID NO: 23 and an amino acid sequence at least 99% identical to the amino acid sequence of SEQ ID NO: 41, and most preferably the amino acid sequence of SEQ ID NO: 23 and the amino acid sequence of SEQ ID NO: 41;
ss. an amino acid sequence at least 85% identical to the amino acid sequence of SEQ ID NO: 42, preferably an amino acid sequence at least 90% identical to the amino acid sequence of SEQ ID NO: 42, more preferably an amino acid sequence at least 95% identical to the amino acid sequence of SEQ ID NO: 42, more preferably an amino acid sequence at least 98% identical to the amino acid sequence of SEQ ID NO: 42, more preferably an amino acid sequence at least 99% identical to the amino acid sequence of SEQ ID NO: 42, and most preferably the amino acid sequence of SEQ ID NO: 42;
tt. an amino acid sequence of a single-domain antibody (sdAb) and an amino acid sequence at least 85% identical to the amino acid sequence of SEQ ID NO: 39, preferably an amino acid sequence of a single-domain antibody (sdAb) and an amino acid sequence at least 90% identical to the amino acid sequence of SEQ ID NO: 39, more preferably an amino acid sequence of a single-domain antibody (sdAb) and an amino acid sequence at least 95% identical to the amino acid sequence of SEQ ID NO: 39, more preferably an amino acid sequence of a single-domain antibody (sdAb) and an amino acid sequence at least 98% identical to the amino acid sequence of SEQ ID NO: 39, more preferably an amino acid sequence of a single-domain antibody (sdAb) and an amino acid sequence at least 99% identical to the amino acid sequence of SEQ ID NO: 39, and most preferably an amino acid sequence of a single-domain antibody (sdAb) and the amino acid sequence of SEQ ID NO: 39;
uu. an amino acid sequence of a single-domain antibody (sdAb) and an amino acid sequence at least 85% identical to the amino acid sequence of SEQ ID NO: 41, preferably an amino acid sequence of a single-domain antibody (sdAb) and an amino acid sequence at least 90% identical to the amino acid sequence of SEQ ID NO: 41, more preferably an amino acid sequence of a single-domain antibody (sdAb) and an amino acid sequence at least 95% identical to the amino acid sequence of SEQ ID NO: 41, more preferably an amino acid sequence of a single-domain antibody (sdAb) and an amino acid sequence at least 98% identical to the amino acid sequence of SEQ ID NO: 41, more preferably an amino acid sequence of a single-domain antibody (sdAb) and an amino acid sequence at least 99% identical to the amino acid sequence of SEQ ID NO: 41, and most preferably an amino acid sequence of a single-domain antibody (sdAb) and the amino acid sequence of SEQ ID NO: 41;
w. an amino acid sequence at least 85% identical to the amino acid sequence of SEQ ID NO: 43 and an amino acid sequence at least 85% identical to the amino acid sequence of SEQ ID NO: 44, preferably an amino acid sequence at least 90% identical to the amino acid sequence of SEQ ID NO: 43 and an amino acid sequence at least 90% identical to the amino acid sequence of SEQ ID NO: 44, more preferably an amino acid sequence at least 95% identical to the amino acid sequence of SEQ ID NO: 43 and an amino acid sequence at least 95% identical to the amino acid sequence of SEQ ID NO: 44, more preferably an amino acid sequence at least 98% identical to the amino acid sequence of SEQ ID NO: 43 and an amino acid sequence at least 98% identical to the amino acid sequence of SEQ ID NO: 44, more preferably an amino acid sequence at least 99% identical to the amino acid sequence of SEQ ID NO: 43 and an amino acid sequence at least 99% identical to the amino acid sequence of SEQ ID NO: 44, and most preferably the amino acid sequence of SEQ ID NO: 43 and the amino acid sequence of SEQ ID NO: 44;
ww. an amino acid sequence at least 85% identical to the amino acid sequence of SEQ ID NO: 43 and an amino acid sequence at least 85% identical to the amino acid sequence of SEQ ID NO: 45, preferably an amino acid sequence at least 90% identical to the amino acid sequence of SEQ ID NO: 43 and an amino acid sequence at least 90% identical to the amino acid sequence of SEQ ID NO: 45, more preferably an amino acid sequence at least 95% identical to the amino acid sequence of SEQ ID NO: 43 and an amino acid sequence at least 95% identical to the amino acid sequence of SEQ ID NO: 45, more preferably an amino acid sequence at least 98% identical to the amino acid sequence of SEQ ID NO: 43 and an amino acid sequence at least 98% identical to the amino acid sequence of SEQ ID NO: 45, more preferably an amino acid sequence at least 99% identical to the amino acid sequence of SEQ ID NO: 43 and an amino acid sequence at least 99% identical to the amino acid sequence of SEQ ID NO: 45, and most preferably the amino acid sequence of SEQ ID NO: 43 and the amino acid sequence of SEQ ID NO: 45;
xx. an amino acid sequence at least 85% identical to the amino acid sequence of SEQ ID NO: 43 and an amino acid sequence at least 85% identical to the amino acid sequence of SEQ ID NO: 46, preferably an amino acid sequence at least 90% identical to the amino acid sequence of SEQ ID NO: 43 and an amino acid sequence at least 90% identical to the amino acid sequence of SEQ ID NO: 46, more preferably an amino acid sequence at least 95% identical to the amino acid sequence of SEQ ID NO: 43 and an amino acid sequence at least 95% identical to the amino acid sequence of SEQ ID NO: 46, more preferably an amino acid sequence at least 98% identical to the amino acid sequence of SEQ ID NO: 43 and an amino acid sequence at least 98% identical to the amino acid sequence of SEQ ID NO: 46, more preferably an amino acid sequence at least 99% identical to the amino acid sequence of SEQ ID NO: 43 and an amino acid sequence at least 99% identical to the amino acid sequence of SEQ ID NO: 46, and most preferably the amino acid sequence of SEQ ID NO: 43 and the amino acid sequence of SEQ ID NO: 46;
yy. an amino acid sequence at least 85% identical to the amino acid sequence of SEQ ID NO: 47 and an amino acid sequence at least 85% identical to the amino acid sequence of SEQ ID NO: 48, preferably an amino acid sequence at least 90% identical to the amino acid sequence of SEQ ID NO: 47 and an amino acid sequence at least 90% identical to the amino acid sequence of SEQ ID NO: 48, more preferably an amino acid sequence at least 95% identical to the amino acid sequence of SEQ ID NO: 47 and an amino acid sequence at least 95% identical to the amino acid sequence of SEQ ID NO: 48, more preferably an amino acid sequence at least 98% identical to the amino acid sequence of SEQ ID NO: 47 and an amino acid sequence at least 98% identical to the amino acid sequence of SEQ ID NO: 48, more preferably an amino acid sequence at least 99% identical to the amino acid sequence of SEQ ID NO: 47 and an amino acid sequence at least 99% identical to the amino acid sequence of SEQ ID NO: 48, and most preferably the amino acid sequence of SEQ ID NO: 47 and the amino acid sequence of SEQ ID NO: 48;
zz. an amino acid sequence at least 85% identical to the amino acid sequence of SEQ ID NO: 47 and an amino acid sequence at least 85% identical to the amino acid sequence of SEQ ID NO: 49, preferably an amino acid sequence at least 90% identical to the amino acid sequence of SEQ ID NO: 47 and an amino acid sequence at least 90% identical to the amino acid sequence of SEQ ID NO: 49, more preferably an amino acid sequence at least 95% identical to the amino acid sequence of SEQ ID NO: 47 and an amino acid sequence at least 95% identical to the amino acid sequence of SEQ ID NO: 49, more preferably an amino acid sequence at least 98% identical to the amino acid sequence of SEQ ID NO: 47 and an amino acid sequence at least 98% identical to the amino acid sequence of SEQ ID NO: 49, more preferably an amino acid sequence at least 99% identical to the amino acid sequence of SEQ ID NO: 47 and an amino acid sequence at least 99% identical to the amino acid sequence of SEQ ID NO: 49, and most preferably the amino acid sequence of SEQ ID NO: 47 and the amino acid sequence of SEQ ID NO: 49;
aaa. an amino acid sequence at least 85% identical to the amino acid sequence of SEQ ID NO: 47 and an amino acid sequence at least 85% identical to the amino acid sequence of SEQ ID NO: 50, preferably an amino acid sequence at least 90% identical to the amino acid sequence of SEQ ID NO: 47 and an amino acid sequence at least 90% identical to the amino acid sequence of SEQ ID NO: 50, more preferably an amino acid sequence at least 95% identical to the amino acid sequence of SEQ ID NO: 47 and an amino acid sequence at least 95% identical to the amino acid sequence of SEQ ID NO: 50, more preferably an amino acid sequence at least 98% identical to the amino acid sequence of SEQ ID NO: 47 and an amino acid sequence at least 98% identical to the amino acid sequence of SEQ ID NO: 50, more preferably an amino acid sequence at least 99% identical to the amino acid sequence of SEQ ID NO: 47 and an amino acid sequence at least 99% identical to the amino acid sequence of SEQ ID NO: 50, and most preferably the amino acid sequence of SEQ ID NO: 47 and the amino acid sequence of SEQ ID NO: 50;
bbb. an amino acid sequence at least 85% identical to the amino acid sequence of SEQ ID NO: 51 and an amino acid sequence at least 85% identical to the amino acid sequence of SEQ ID NO: 52, preferably an amino acid sequence at least 90% identical to the amino acid sequence of SEQ ID NO: 51 and an amino acid sequence at least 90% identical to the amino acid sequence of SEQ ID NO: 52, more preferably an amino acid sequence at least 95% identical to the amino acid sequence of SEQ ID NO: 51 and an amino acid sequence at least 95% identical to the amino acid sequence of SEQ ID NO: 52, more preferably an amino acid sequence at least 98% identical to the amino acid sequence of SEQ ID NO: 51 and an amino acid sequence at least 98% identical to the amino acid sequence of SEQ ID NO: 52, more preferably an amino acid sequence at least 99% identical to the amino acid sequence of SEQ ID NO: 51 and an amino acid sequence at least 99% identical to the amino acid sequence of SEQ ID NO: 52, and most preferably the amino acid sequence of SEQ ID NO: 51 and the amino acid sequence of SEQ ID NO: 52;
ccc. an amino acid sequence at least 85% identical to the amino acid sequence of SEQ ID NO: 51 and an amino acid sequence at least 85% identical to the amino acid sequence of SEQ ID NO: 53, preferably an amino acid sequence at least 90% identical to the amino acid sequence of SEQ ID NO: 51 and an amino acid sequence at least 90% identical to the amino acid sequence of SEQ ID NO: 53, more preferably an amino acid sequence at least 95% identical to the amino acid sequence of SEQ ID NO: 51 and an amino acid sequence at least 95% identical to the amino acid sequence of SEQ ID NO: 53, more preferably an amino acid sequence at least 98% identical to the amino acid sequence of SEQ ID NO: 51 and an amino acid sequence at least 98% identical to the amino acid sequence of SEQ ID NO: 53, more preferably an amino acid sequence at least 99% identical to the amino acid sequence of SEQ ID NO: 51 and an amino acid sequence at least 99% identical to the amino acid sequence of SEQ ID NO: 53, and most preferably the amino acid sequence of SEQ ID NO: 51 and the amino acid sequence of SEQ ID NO: 53;
ddd. an amino acid sequence at least 85% identical to the amino acid sequence of SEQ ID NO: 51 and an amino acid sequence at least 85% identical to the amino acid sequence of SEQ ID NO: 54, preferably an amino acid sequence at least 90% identical to the amino acid sequence of SEQ ID NO: 51 and an amino acid sequence at least 90% identical to the amino acid sequence of SEQ ID NO: 54, more preferably an amino acid sequence at least 95% identical to the amino acid sequence of SEQ ID NO: 51 and an amino acid sequence at least 95% identical to the amino acid sequence of SEQ ID NO: 54, more preferably an amino acid sequence at least 98% identical to the amino acid sequence of SEQ ID NO: 51 and an amino acid sequence at least 98% identical to the amino acid sequence of SEQ ID NO: 54, more preferably an amino acid sequence at least 99% identical to the amino acid sequence of SEQ ID NO: 51 and an amino acid sequence at least 99% identical to the amino acid sequence of SEQ ID NO: 54, and most preferably the amino acid sequence of SEQ ID NO: 51 and the amino acid sequence of SEQ ID NO: 54;
eee. an amino acid sequence at least 85% identical to the amino acid sequence of SEQ ID NO: 55 and an amino acid sequence at least 85% identical to the amino acid sequence of SEQ ID NO: 56, preferably an amino acid sequence at least 90% identical to the amino acid sequence of SEQ ID NO: 55 and an amino acid sequence at least 90% identical to the amino acid sequence of SEQ ID NO: 56, more preferably an amino acid sequence at least 95% identical to the amino acid sequence of SEQ ID NO: 55 and an amino acid sequence at least 95% identical to the amino acid sequence of SEQ ID NO: 56, more preferably an amino acid sequence at least 98% identical to the amino acid sequence of SEQ ID NO: 55 and an amino acid sequence at least 98% identical to the amino acid sequence of SEQ ID NO: 56, more preferably an amino acid sequence at least 99% identical to the amino acid sequence of SEQ ID NO: 55 and an amino acid sequence at least 99% identical to the amino acid sequence of SEQ ID NO: 56, and most preferably the amino acid sequence of SEQ ID NO: 55 and the amino acid sequence of SEQ ID NO: 56;
fff. an amino acid sequence at least 85% identical to the amino acid sequence of SEQ ID NO: 57, preferably an amino acid sequence at least 90% identical to the amino acid sequence of SEQ ID NO: 57, more preferably an amino acid sequence at least 95% identical to the amino acid sequence of SEQ ID NO: 57, more preferably an amino acid sequence at least 98% identical to the amino acid sequence of SEQ ID NO: 57, more preferably an amino acid sequence at least 99% identical to the amino acid sequence of SEQ ID NO: 57, and most preferably the amino acid sequence of SEQ ID NO: 57; or
ggg. an amino acid sequence at least 85% identical to the amino acid sequence of SEQ ID NO: 58, preferably an amino acid sequence at least 90% identical to the amino acid sequence of SEQ ID NO: 58, more preferably an amino acid sequence at least 95% identical to the amino acid sequence of SEQ ID NO: 58, more preferably an amino acid sequence at least 98% identical to the amino acid sequence of SEQ ID NO: 58, more preferably an amino acid sequence at least 99% identical to the amino acid sequence of SEQ ID NO: 58, and most preferably the amino acid sequence of SEQ ID NO: 58.

24. The construct according to any one of the preceding claims, further comprising an N-terminal amino acid sequence comprising one or two of the following ((a) and (b)): (a) a leader sequence comprising the amino acid sequence of SEQ ID NO: 30, optionally followed by a first linker sequence, and (b) a flag tag comprising the amino acid sequence of SEQ ID NO: 61, optionally followed by a second linker sequence.

25. A pharmaceutical composition comprising the construct according to any one of the preceding claims.

26. A pharmaceutical composition according to claim 25 or a construct according to any one of claims 1-12, for use in the treatment of cancer.

27. The pharmaceutical composition or construct for use according to claim 26, wherein the cancer is a cancer expressing CD40.

28. A nucleic acid, or a set of nucleic acids, encoding the construct of any one of claims 1-24.

29. A recombinant cell containing a nucleic acid, or a set of nucleic acids, according to claim 28 and expressing the construct of any one of claims 1-24.

30. A method for producing a construct of any one of claims 1-24, the method comprising expressing said construct in a recombinant cell according to claim 29 from the nucleic acid or set of nucleic acids according to claim 28, wherein the method optionally further comprises purifying and formulating said construct into a pharmaceutical composition according to claim 25.
